(19) United States
(12) Patent Application Publication
(10) Pub. No.: US 2026/0116990 A1
(43) Pub. Date: Apr. 30, 2026

(54) VHH ANTIBODIES AND USES THEREOF

(71) Applicant: KEY2BRAIN AB, Solna (SE)

(72) Inventors: Elisabet OHLIN SJÖSTRÖM, Stockholm (SE); Magnus BERGLUND, Vendelsö (SE); Erik NORDLING, Danderyd (SE); Mikael ÅSTRAND, Sollentuna (SE)

(21) Appl. No.: 19/132,619

(22) PCT Filed: Nov. 22, 2023
(86) PCT No.: PCT/SE2023/051180

§ 371 Date: May 23, 2025

(30) Foreign Application Priority Data
Nov. 23, 2022 (SE) 2251367-5

(51) Int. Cl. C07K 16/28; A61K 45/06; A61K 47/68; A61K 49/00; A61P 25/00; C07K 16/1271

(52) U.S. Cl. CPC C07K 16/2881; A61K 45/06; A61K 47/6891; A61K 49/00; A61P 25/00; C07K 16/1271; C07K 2317/22; C07K 2317/24; C07K 2317/31; C07K 2317/565; C07K 2317/567; C07K 2317/569; C07K 2317/622; C07K 2317/92; C07K 2319/00

(57) ABSTRACT

The present invention relates to variable domain of heavy chain-only (VHH) antibody binding specifically to the transferrin receptor 1 (TfR1) and uses thereof to transport therapeutic agents or imaging agents into cells and over the blood-brain barrier.

### TECHNICAL FIELD

The invention particularly relates to variable domain of heavy chain-only (VHH) antibodies that are able to bind the transferrin receptor, and to the use of such VHH antibodies to transport molecules across the blood-brain barrier to relevant targets in the brain.

### BACKGROUND

Brain exposure of drugs to target diseases of the central nervous system (CNS) is inherently difficult since the blood-brain barrier (BBB) protects the brain from unwanted substances present in the peripheral circulation, including antibodies and other proteins that may have a therapeutic effect within the brain. There are also small molecules that due to their properties, such as lack of lipophilicity or substrates to efflux pumps, are excluded from the brain compartment. Normally, a compound with a size under 600 Daltons (Da) can pass the BBB if not excluded by other forces. Larger molecules, such as proteins, do not readily pass into the brain in any considerable amounts if not helped by specific active transport. For therapeutic antibodies and protein-based drugs less than 0.1% of systemically injected therapeutic antibodies is estimated to reach the brain compartment. Several strategies to overcome this tight barrier have been tested and evaluated.

The BBB is composed of brain endothelial cells (BECs) as a first obstacle to entering the brain. Other cells of the so-called neurovascular unit (NVU) are also of importance for the transport and interplay to target cells in the brain parenchyma. In the human brain, the vessels of the brain span a total of 20 m², representing a large surface area, which can present a circulating therapeutic an opportunity for brain exposure.

Therapeutic antibodies or other protein-based drugs have a great potential to treat pathologies of the CNS. However, the low availability of such therapeutic molecules in the brain compartment is a major problem. Recently, therapeutic monoclonal antibodies with targets within the brain, such as amyloid beta protofibrils, have reported clinical effect. Nonetheless, the exposure in the human brain compartment of these therapeutic molecules after each administration is not regarded to be at their optimum.

There is, thus, a need to increase exposure of therapeutic molecules to the brain in order to improve the safety, dosage and total costs of CNS therapeutics.

Receptor-mediated transcytosis, a natural mechanism using endogenous receptors expressed at the luminal surface of the BBB, has been reported to be successful in increasing brain exposure of therapeutic molecules as well as being clinically efficacious and safe.

WO 2020/144233 discloses variable domain of camelid heavy chain-only (VHH) molecules, which bind the transferrin receptor (TfR) and uses thereof to transport molecules of pharmaceutical or diagnostic interest into cells and in organs, in pathological conditions including cancer.

WO 2016/077840, WO 2019/089395, WO 2020/056327 and WO 2022/103769 disclose TfR-specific binding moieties that can be used to carry biomolecules across membranes, including the BBB and the gastrointestinal tract. These TfR-specific binding moieties include single domain nurse shark variable domain of new antigen receptor (VNAR) antibodies that bind to TfR.

WO 2016/081643 relates to anti-transferrin receptor antibodies and methods of using the same.

There is still a need for efficacious transporters of therapeutic molecules into the brain. These transporters should be able to conjugate or fuse to a therapeutic or diagnostic molecule in a manner that does not impact the target engagement of the transporter or the therapeutic efficacy of the therapeutic molecule.

### SUMMARY

It is a general objective to provide VHH molecules specific to the transferrin receptor 1 and that can effectively transport cargo through receptor-mediated transcytosis into a desired compartment.

These and other objectives are met by embodiments of the invention.

The present invention is defined in the independent claims. Further embodiments of the invention are defined in the dependent claims.

An aspect of the invention relates to a variable domain of heavy chain-only (VHH) antibody binding specifically to a transferrin receptor 1 (TfR1). The VHH antibody comprises a complementarity determining region 1 (CDR1) consisting of the amino acid sequence GX₁X₂FX₃X₄X₅Y, wherein X₁ is T or N, X₂ is P, D or R, X₃ is S, T or G, X₄ is N, L, M or I, and X₅ is N or E. The VHH antibody also comprises a CDR2 consisting of the amino acid sequence FTX₆X₇GX₈T as defined in SEQ ID NO: 1, wherein X₆ is S, T or A, X₇ is G, A, H, S or T, and X₈ is S, N or D. The VHH antibody further comprises a CDR3 consisting of the amino acid sequence X₉X₁₀LX₁₁X₁₂, wherein X₉ is Y or H, X₁₀ is F or Y, X₁₁ is D or G, and X₁₂ is V, N or D.

Another aspect of the invention relates to a VHH antibody binding specifically to a TfR1. The VHH antibody comprises a CDR1 consisting of the amino acid sequence DSAFX₂₈MNT as defined in SEQ ID NO: 38, wherein X₂₈ is S or N. The VHH antibody also comprises a CDR2 consisting of the amino acid sequence IVSDDNT as defined in SEQ ID NO: 39. The VHH further comprises a CDR3 consisting of the amino acid sequence KGDVV as defined in SEQ ID NO: 40.

A further aspect of the invention relates to a fusion molecule comprising a VHH antibody according above, linked to at least one molecule.

Related aspects of the invention define a fusion molecule according to above for use as a medicament, wherein the at least one molecule is a therapeutic agent, or for use in treatment of a central nervous system (CNS) disease or disorder, wherein the at least one molecule is a therapeutic agent capable of treating the CNS disease or disorder.

Yet another aspect of the invention relates to a pharmaceutical composition comprising a fusion molecule according to above and a pharmaceutically acceptable vehicle. In such a pharmaceutical composition, the at least one molecule is a therapeutic agent.

Other aspects of the invention relate to a nucleic acid molecule encoding a VHH antibody or a fusion molecule according to above, an expression vector comprising a nucleic acid molecule according to above operably linked to a promoter, and a host cell comprising a nucleic acid molecule according to above or an expression vector according to above.

The VHH antibodies of the present invention bind specifically to the TfR1 without interfering with the binding of transferrin to the TfR1. The binding properties of the VHH antibodies are tailored to be optimal for transcytosis over endothelial cells at the BBB. The VHH antibodies can thereby be used as transporters for various molecules, including therapeutic agents or diagnostic imaging agents, into the brain compartment when administered systemically.

### BRIEF DESCRIPTION OF THE DRAWINGS

The embodiments, together with further objects and advantages thereof, may best be understood by making reference to the following description taken together with the accompanying drawings, in which:

FIG. 1. Strategy for establishment of TfR1-binding VHHs

Graphical illustration of llama immunizations with immunogens (1A). Immunization of llama (Llama glama), N=2, was performed according to the ModiPhage™ method at Modiquest Research BV (the Netherlands). Primary immunization was performed with an ectodomain of human transferrin receptor 1 (hTfR1) (500 μg protein+Complete Freund's Adjuvant (CFA), intramuscular (i.m.) on Day 1. Boost doses (500 μg protein+Incomplete Freund's Adjuvant (IFA), i.m.) were given on Day 21 of mouse TfR1 (mTfR1) and Day 42 of hTfR1. On Day 54, peripheral blood withdrawal was performed to study immune response with enzyme-linked immunosorbent assay (ELISA), testing for presence of both immunoglobulin G1 (IgG1) and IgG2/3 antibodies and their reactivity towards mTfR1 and hTfR1. Immunization was repeated on Day 86 (hTfR1+IFA) and Day 107 (hTfR1+IFA). On Day 117 a new peripheral blood sample was analyzed. An additional pre-harvest boost dose with a 1:1 mixture of hTfR1 and mTfR1 (250 μg+250 μg+IFA) was given on Day 120 followed by peripheral blood lymphocyte (PBL) harvest on Day 124.

Graphical overview of phage library establishment (1B). PBLs were isolated by density gradient centrifugation using Ficoll-Paque™ PLUS ˜1.5×10⁹. Ribonucleic acid (RNA) was extracted followed by reverse transcription to complimentary deoxyribonucleic acid (cDNA). cDNA was then used as a template for a polymerase chain reaction (PCR) to amplify the IgG repertoire (IgG2/3, from variable heavy (VH) domain to constant heavy chain 2 (CH2), heavy chain only antibodies). A first PCR reaction to amplify all antibodies (IgG1 and IgG2/3) and a second nested PCR reaction were performed to amplify and isolate the VHH repertoire (as described in in Pardon et al., A general protocol for the generation of Nanobodies for structural biology. Nat Protoc 9:674-693 (2014). Vector digestion was followed by DNA amplification as described above and a total of 1600 ng DNA was used to electroporate TG1 Escherichia coli in yielding a library of estimated size 3.2× 10⁸.

FIG. 2. Graphical representations of VHH and various VHH-containing fusion proteins

FIG. 2 schematically illustrates a VHH monomer and various VHH-containing fusion proteins. Free VHHs bind as monomers to one binding site on TfR1. A bivalent VHH fusion protein was formed between two VHHs and a human fragment crystallizable (Fc) region. Functional monovalent fusion proteins were produced between one VHH and a single-chain variable fragment (scFv) or between one VHH and a non-antibody-derived molecule (X). FIG. 2 also shows a bivalent VHH fusion protein with two non-antibody-derived molecules (X).

FIG. 3. Affinity measurements of monomeric VHHs binding to hTfR1 using SPR

The figure shows representative sensorgrams of KB_B01 (3A) at increasing concentrations ranging from 6.25 to 100 nM binding to hTfR1 loaded on human transferrin (hTf) immobilized to a dextran coated gold (CM5) chip (Cytiva) by amine coupling. As a control benchmark, full-length monoclonal antibody BA1 (3B) was run at the same concentrations.

FIG. 4. Affinity measurements of dimeric VHH-Fc proteins binding to hTfR1, cTfR1 and mTfR1 using SPR

The figure shows representative sensorgrams of hTfR1 (A, D and G), cynomolgus TfR1 (cTfR1) (B, E and H) or mTfR1 (C, F and I) at increasing concentrations ranging from 0.16 to 100 nM binding to Fc-fused KB_B01 (4A-4C), BV (4D-4F), and benchmark, full-length monoclonal antibody BA2 (4G-4I) loaded on a protein A coated chip (Cytiva).

FIG. 5. Affinity measurements of dimeric KB_B01-Fc to hTfR1 and cTfR1 in the absence and presence of hTf

The figure shows representative sensorgrams of KB_B01-Fc (5A-5B), benchmark BV-Fc (5C-D) and BA2 (5E-5F) binding to hTfR1 in the absence (5A, 5C and 5E) and presence (5B, 5D and 5F) of an excess concentration of 250 nM hTf.

FIG. 6. Sequence alignments of closely related sequences to KB_B01

The figure shows amino acid sequences of KB_B01 (SEQ ID NO: 30) and closely related clones KB_B02 to KB_B10 and benchmark VHH BV in single letter code. Dots indicate amino acids identical to reference sequence (KB_B01) and boxes indicate the three complementarity-determining regions (CDRs).

FIG. 7. Affinity measurements of fusion proteins of VHH and scFv binding to hTfR1 using SPR

The figure shows representative sensorgrams of hTfR1 amine coupled to the surface of a CM5 chip and exposed to KB_B01 genetically fused to a scFv at the C-terminus of the VL (scFv-VHH, 7A) or at the N-terminus of the VH (VHH-scFv, 7B). Exposure at increasing concentrations ranging from 0.25 to 64 nM in 1:4 step increments. Benchmark VHH BV was also fused to either end of the same scFv (scFv-VHH, 7C) and (VHH-scFv, 7D) and tested at concentrations ranging from 0.08 to 50 nM in 1:5 step increments.

FIG. 8. Cellular uptake via hTfR1 in HEK293T cells

HEK293T cells were cultured until confluency (4-5 days) in 96 well plates. KB_B01-Fc, BV-Fc or BA1 and negative control (hTfR1 inert VHH-Fc) were added at 20 nM or 5 nM (diluted in DMEM) and incubated for 40 and 120 minutes, respectively, and then rinsed by PBS and fixed in 4% PFA. For the longer incubation time, replacement of medium was performed at t=30 minutes by incubation with fresh DMEM. After fixation, cells were immunostained and analyzed with confocal microscopy. Shown here are representative images of cellular uptake after 40 minutes at 20 nM for KB_B01-Fc, BV-Fc and BA1 and a negative control, or at 5 nM for 120 minutes incubation for KB_B01-Fc, BV-Fc and BA1. The figure also shows absence of signal using the non-hTfR1-binding VHH-Fc negative control. Images are shown as 8-bit grayscale. Scale bar 50 μM.

FIG. 9. Brain and blood distribution after systemic administration of VHH-Fc fusions.

Radiolabeled [¹²⁵I]VHH-Fc fusion proteins were injected as described in Example VIII. Brain concentrations (9A), and blood concentrations (9B) are shown. Blood samples were taken at t=30 minutes, t=1 hour and terminally (at 2.5 hours). At 2.5 hours after injection, animals were euthanized, transcardially perfused with NaCl and brains were excised and analyzed for radioactivity. All concentrations are expressed as % of injected dose (radioactivity) and for brain normalized to body weight (standardized uptake value SUV).

FIG. 10. Brain distribution and biodistribution after systemic administration of VHH-scFvs

Radiolabeled [¹²⁵I]VHH-scFv fusion proteins were injected as described in Example VIII. Brain exposure was analyzed in both wild type mice, and a mouse model of Alzheimer's disease (AD), APP^{NL-G-F} named “nlgf” in the graphics. Brain concentrations as SUV at 2 hours after injection of KB_B01-scFv and BV-scFv fused to the N-terminus of 3D6 scFv as described in Example IV (10A). The dynamics of brain retention in APP^{NL-G-F} mice vs WT mice at 2, 6 and 24 hours after injection with KB_B01-scFv (10B). Brain (10C) and blood (10C) concentrations 24 hours after injection. One group of APP^{NL-G-F} animals were also injected with negative control VHH-scFv fusion terminated at 24 hours, as well as KB_B01-scFv and BV-scFv. Retention in AD mice vs WT is read from each construct.

FIG. 11. Vascular versus parenchymal distribution of scFv-VHH fusions

As described in Example IX, one hemisphere from brains of mice injected with VHH-scFvs (as described in Example VIII) were cryosectioned. Representative sections were first immunostained with the vascular marker CD31 and then immersed into Nuclear Track Emulsion (NTE), followed by a long development in cold room. After this, images of both CD31 and NTE were acquired using a widefield Zeiss Observer Microscope. Overlay images were made as binary 8-bit images (11A). From these images, NTE white dots shows localization of ¹²⁵I-protein and its relation to the blood vessels. For clarity, blood vessel immunostaining is shown in the bottom panel and an overlay in the top panel, where blood vessel profiles are shown as thin outlines (white lines). Scale bar 20 μm. A quantification was made from 20 frames from the same brain as shown in the microphotographs of relative distribution in blood vessel profiles or in the parenchyma (11B). Values shown as Mean+−standard deviation (SD).

FIG. 12. Brain and blood distribution after systemic administration of VHH-Fc fusion KB_B012 in TfR1 extracellular domain-humanized mice

Radiolabeled [¹²⁵I]VHH-Fc fusion protein KB_B03 (KB_B12-Fc) was injected as described in Example XI in hTfR1-homozygous (HOM), heterozygous (HET) and wild type (WT) genotype. (12A) Blood concentrations over time are shown. Blood samples were taken at t=5 minutes, t=30 minutes, t=1 hour and terminally (at 2.0 hours). (12B) Distribution in the blood compartment between while blood, plasma and pellet. (12C) Brain concentrations at 2.0 hours. At 2 hours after injection, animals were euthanized, transcardially perfused with NaCl and brains were excised and analyzed for radioactivity. Blood concentrations are expressed as (% of injected dose, radioactivity), while brain uptake is expressed as standardized uptake value, SUV (% of injected dose corrected for animal body weight).

### DETAILED DESCRIPTION

The invention relates to variable domain of heavy chain-only (VHH) antibodies, also referred to as single-domain antibodies, and in particular to such VHH antibodies that are able to bind the transferrin (Tf) receptor 1 (TfR1), and to the use of such VHH antibodies to transport molecules across the blood-brain barrier (BBB).

Brain exposure of drugs targeting diseases of the central nervous system (CNS) is inherently difficult since the BBB protects the brain from unwanted substances present in the peripheral circulation, including antibodies, proteins and other molecules that may have a therapeutic effect within the brain. Receptor-mediated transcytosis, a natural mechanism using endogenous receptors expressed at the luminal surface of the BBB, has been suggested to increase BBB exposure of drugs. One such endogenous receptor that could be used to achieve receptor-mediated transcytosis is the transferrin receptor.

The transferrin receptor is a carrier protein for transferrin, which is a glycoprotein that binds to and mediates transport of iron (Fe) through the blood plasma. The transferrin receptor imports iron by internalizing the transferrin-iron complex through receptor-mediated endocytosis. In humans and other mammals, there are two transferring receptors: transferrin receptor 1 (TfR1) and transferrin receptor 2 (TfR2). TfR1 is a high affinity ubiquitously expressed receptor while expression of TfR2 is restricted to certain cell types and is unaffected by intracellular iron concentrations. TfR2 binds to transferrin with a 25- to 30-fold lower affinity than what TfR1 does. Transferrin receptor, as used herein refers to the TfR1 homologue, also known as Cluster of Differentiation 71 (CD71), which is encoded by the TFRC gene in humans.

Extracellular domains of human TfR1 (hTfR1) and mouse TfR1 (mTfR1) were used to immunize llama (Llama glama) animals and create a library of VHH antibody expressing clones. These VHH antibodies were screened for binding to hTfR1 and a key VHH antibody KB_B01 having desired binding properties was selected. Further VHH antibodies were identified showing sequence similarities to KB_B01. The VHH antibodies of the invention bind specifically to hTfR1 and also mTfR1 with binding characteristics suitable for endocytosis, including binding affinity and binding to hTfR1 without interfering with binding of transferrin (Tf) to hTfR1. As shown in the experimental section, these VHH antibodies retain their TfR1 binding characteristics when presented as VHH-containing fusion proteins, including bivalent VHH fusion proteins formed between two VHH antibodies and human constant antibody fragment crystallizable region (Fc) and functional fusion proteins fused to a drug molecule, such as a single-chain variable fragment (scFv). Experimental data further show that the VHH antibodies, including VHH-containing fusion proteins, were taken up by human cells expressing hTfR1 and were transcytosed through a human brain-like endothelial cell monolayer used as an in vitro model of the BBB. The distribution of VHH antibodies in the brain was verified in in vivo and showed a brain distribution than benchmark antibodies, including benchmark VHH antibodies.

The VHH antibodies of the invention further have advantages as compared to VHH antibodies as disclosed in WO 2020/144233. Firstly, the distribution of fusion proteins between the VHH antibodies and a scFv molecule to the brain parenchyma without entrapment in the blood vessels was superior as compared to a fusion protein between a VHH antibody of WO 2020/144233 and the same scFv molecule. In more detail, the fusion protein with VHH antibodies of the invention had a preferential distribution to brain parenchyma of close to 80%, whereas the corresponding number for the VHH antibody of WO 2020/144233 was merely slightly above 50%. Another advantage of the VHH antibodies of the invention over the VHH antibody of WO 2020/144233 is that the VHH antibodies of the invention have a higher brain-to-blood ratio as compared to negative control. This means that the VHH antibodies of the invention are suitable for specific targeting of disease-related proteins in the brain with less unspecific binding in other compartments in the body.

An aspect of the invention relates to a variable domain of heavy chain-only (VHH) antibody binding specifically to a transferrin receptor 1 (TfR1). The VHH antibody comprises a complementarity determining region 1 (CDR1) consisting of the amino acid sequence GX₁X₂FX₃X₄X₅Y, wherein X₁ is T or N, X₂ is P, D or R, X₃ is S, T or G, X₄ is N, L, M or I, and X₅ is N or E. The VHH antibody also comprises a CDR2 consisting of the amino acid sequence FTX₆X₇GX₈T as defined in SEQ ID NO: 1, wherein X₆ is S, T or A, X₇ is G, A, H, S or T, and X₈ is S, N or D. The VHH antibody further comprises a CDR3 consisting of the amino acid sequence X₉X₁₀LX₁₁X₁₂, wherein X₉ is Y or H, X₁₀ is F or Y, X₁₁ is D or G, and X₁₂ is V, N or D.

In an embodiment, the CDR1 consists of the amino acid sequence GTX₂FX₃X₄NY as defined in SEQ ID NO: 2, wherein X₂ is P or D, X₃ is S or T, and X₄ is N, L or M. In this embodiment, the CDR3 consists of the amino acid sequence X₉FLX₁₁X₁₂, wherein X₉ is Y or H, X₁₁ is D or G, and X₁₂ is V, N or D.

In an embodiment, the CDR1 consists of the amino acid sequence GTDFSX₄NY as defined in SEQ ID NO: 3, wherein X₄ is L or M. In this embodiment, the CDR2 consists of the amino acid sequence FTX₆X₇GST as defined in SEQ ID NO: 4, wherein X₆ is S or A, and X₇ is S or T. In this embodiment, the CDR3 consists of the amino acid sequence HFLGD as defined in SEQ ID NO: 5.

In an embodiment, the CDR1 consists of the amino acid sequence GTDFSLNY as defined in SEQ ID NO: 6. In this embodiment, the CDR2 consists of the amino acid sequence FTAX₇GST as defined in SEQ ID NO: 7, wherein X₇ is S or T. In this embodiment, the CDR3 consists of the amino acid sequence HFLGD as defined in SEQ ID NO: 5.

In a particular embodiment, the CDR1 consists of the amino acid sequence GTDFSLNY as defined in SEQ ID NO: 6, the CDR2 consists of the amino acid sequence FTATGST as defined in SEQ ID NO: 8 and the CDR3 consists of the amino acid sequence HFLGD as defined in SEQ ID NO: 5. A VHH antibody having CDR regions according to this particular embodiment is denoted KB_B06 herein.

In another particular embodiment, the CDR1 consists of the amino acid sequence GTDFSLNY as defined in SEQ ID NO: 6, the CDR2 consists of the amino acid sequence FTASGST as defined in SEQ ID NO: 9 and the CDR3 consists of the amino acid sequence HFLGD as defined in SEQ ID NO: 5. A VHH antibody having CDR regions according to this particular embodiment is denoted KB_B07 herein.

In a further particular embodiment, the CDR1 consists of the amino acid sequence GTDFSMNY as defined in SEQ ID NO: 10, the CDR2 consists of the amino acid sequence FTSTGST as defined in SEQ ID NO: 11 and the CDR3 consists of the amino acid sequence HFLGD as defined in SEQ ID NO: 5. A VHH antibody having CDR regions according to this particular embodiment is denoted KB_B05 herein.

In an embodiment, the CDR1 consists of the amino acid sequence GTX₂FX₃X₄NY as defined in SEQ ID NO: 2, wherein X₂ is P or D, X₃ is S or T, and X₄ is N, L or M. In this embodiment, the CDR2 consists of the amino acid sequence FTX₆X₇GX₈T as defined in SEQ ID NO: 1, wherein X₆ is S or T, X₇ is G, A, H or S, and X₈ is S, N or D. In this embodiment, the CDR3 consists of the amino acid sequence YFLDX₁₂ as defined in SEQ ID NO: 12, wherein X₁₂ is V or N.

In an embodiment, the CDR1 consists of the amino acid sequence GTPFX₃NNY as defined in SEQ ID NO: 13, wherein X₃ is S or T. In this embodiment, the CDR2 consists of the amino acid sequence FTSX₇GX₈T as defined in SEQ ID NO: 14, wherein X₇ is G or A, and X₈ is S or N. In this embodiment, the CDR3 consists of the amino acid sequence YFLDV as defined in SEQ ID NO: 15.

In a particular embodiment, the CDR1 consists of the amino acid sequence GTPFSNNY as defined in SEQ ID NO: 16, the CDR2 consists of the amino acid sequence FTSGGST as defined in SEQ ID NO: 17 and the CDR3 consists of the amino acid sequence YFLDV as defined in SEQ ID NO: 15. A VHH antibody having CDR regions according to this particular embodiment is denoted KB_B01 herein.

In another particular embodiment, the CDR1 consists of the amino acid sequence GTPFTNNY as defined in SEQ ID NO: 18, the CDR2 consists of the amino acid sequence FTSAGNT as defined in SEQ ID NO: 19 and the CDR3 consists of the amino acid sequence YFLDV as defined in SEQ ID NO: 15. A VHH antibody having CDR regions according to this particular embodiment is denoted KB_B02 herein.

In an embodiment, the CDR1 consists of the amino acid sequence GTDFSX₄NY as defined in SEQ ID NO: 3, wherein X₄ is L or M. In this embodiment, the CDR2 consists of the amino acid sequence FTX₆X₇GX₈T as defined in SEQ ID NO: 1, wherein X₆ is S or T, X₇ is H or S, and X₈ is N or D. In this embodiment, the CDR3 consists of the amino acid sequence YFLDN as defined in SEQ ID NO: 20.

In a particular embodiment, the CDR1 consists of the amino acid sequence GTDFSLNY as defined in SEQ ID NO: 6, the CDR2 consists of the amino acid sequence FTTHGDT as defined in SEQ ID NO: 21 and the CDR3 consists of the amino acid sequence YFLDN as defined in SEQ ID NO: 20. A VHH antibody having CDR regions according to this particular embodiment is denoted KB_B03 herein.

In another particular embodiment, the CDR1 consists of the amino acid sequence GTDFSMNY as defined in SEQ ID NO: 10, the CDR2 consists of the amino acid sequence FTSSGNT as defined in SEQ ID NO: 22 and the CDR3 consists of the amino acid sequence YFLDN as defined in SEQ ID NO: 20. A VHH antibody having CDR regions according to this particular embodiment is denoted KB_B04 herein.

In a further particular embodiment, the CDR1 consists of the amino acid sequence GNRFGIEY as defined in SEQ ID NO: 23, the CDR2 consists of the amino acid sequence FTSAGST as defined in SEQ ID NO: 24 and the CDR3 consists of the amino acid sequence HYLGD as defined in SEQ ID NO: 25. A VHH antibody having CDR regions according to this particular embodiment is denoted KB_B08 herein.

In an embodiment, the VHH antibody is of a formula: framework region 1 (FR1)-CDR1-FR2-CDR2-FR3-CDR3-FR4.

In an embodiment, the FR1 has an amino acid sequence QVQLQESGX₁₄GX₁₅VQX₁₆GGSLRLSCX₁₇X₁₈S as defined in SEQ ID NO: 26, wherein X₁₄ is R or G, X₁₅ is L or V, X₁₆ is A, or T, X₁₇ is A, T or V, and X₁₈ is A, L or V.

In a particular embodiment, the FR1 has an amino acid sequence QVQLQESGGGLVQX₁₆GGSLRLSCX₁₇X₁₈S as defined in SEQ ID NO: 77 wherein X₁₆ is A, or T, X₁₇ is A, T or V, and X₁₈ is A, L or V.

In an embodiment, the FR2 has an amino acid sequence X₁₉RWYRQAPGX₂₀QREX₂₁VAG as defined in SEQ ID NO: 27, wherein X₁₉ is M or L, X₂₀ is K or N, and X₂₁ is W or F.

In a particular embodiment, the FR2 has an amino acid sequence X₁₉RWYRQAPGX₂₀QREWVAG as defined in SEQ ID NO: 78 wherein X₁₉ is M or L, and X₂₀ is K or N.

In an embodiment, the FR3 has an amino acid sequence NYX₂₀DSX₂₁KGRFTIX₂₂RDNAX₂₃X₂₄TVYLQMDX₂₅LX₂₆PEDTAVYX₂₇C as defined in SEQ ID NO: 28, wherein X₂₀ is A, P or G, X₂₁ is V or M, X₂₂ is S or G, X₂₃ is E or K, X₂₄ is G or N, X₂₅ is N or S, X₂₆ is K, I or T, and X₂₇ is Y or F.

In a particular embodiment, the FR3 has an amino acid sequence NYX₂₀DSX₂₁KGRFTIX₂₂RDNAX₂₃X₂₄TVYLQMDX₂₅LX₂₆PEDTAVYX₂₇C as defined in SEQ ID NO: 28, wherein X₂₀ is A or P, X₂₁ is V or M, X₂₂ is S or G, X₂₃ is E or K, X₂₄ is G or N, X₂₅ is N or S, X₂₆ is K or T, and X₂₇ is Y or F.

In an embodiment, FR4 has an amino acid sequence WGQGTQVTVSS as defined in SEQ ID NO: 29.

In a particular embodiment, the VHH antibody has amino acid sequences of FR1, FR2, FR3 and FR4 as defined in SEQ ID NO: 26 or 77, 27 or 78, 28 and 29.

The present invention also encompasses that the VHH antibody has an amino acid sequence of FR1 comprising or consisting of SEQ ID NO: 26 or 77, or a variant thereof having at least 88% sequence identity to SEQ ID NO: 26 or 77, preferably at least 92% sequence identity, and more preferably at least 96% sequence identity.

The present invention also encompasses that the VHH antibody has an amino acid sequence of FR2 comprising or consisting of SEQ ID NO: 27 or 78, or a variant thereof having at least 82% sequence identity to SEQ ID NO: 27 or 78, preferably at least 88% sequence identity, and more preferably at least 94% sequence identity.

The present invention also encompasses that the VHH antibody has an amino acid sequence of FR3 comprising or consisting of SEQ ID NO: 28, or a variant thereof having at least 92% sequence identity to SEQ ID NO: 28, preferably at least 94% sequence identity, and more preferably at least 97% sequence identity.

The present invention also encompasses that the VHH antibody has an amino acid sequence of FR4 comprising or consisting of SEQ ID NO: 29, or a variant thereof having at least 72% sequence identity to SEQ ID NO: 29, preferably at least 81% sequence identity, and more preferably at least 90% sequence identity.

As used herein, the term “% sequence identity” may be determined using methods well known in the art. For example, % sequence identity be calculated as follows. The query sequence is aligned to the target sequence using the CLUSTAL W algorithm. A comparison is made over the window corresponding to the shortest of the aligned sequences. The shortest of the aligned sequences may in some instances be the target sequence. In other instances, the query sequence may constitute the shortest of the aligned sequences. The amino acid residues at each position are compared and the percentage of positions in the query sequence that have identical correspondences in the target sequence is reported as % sequence identity.

An amino acid sequence having a defined % sequence identity of a reference amino acid sequence is preferably obtained by amino acid substitutions, such as by conservative amino acid replacements. Conservative amino acid replacements, also denoted as conservative amino acid substitutions or mutations, is an amino acid replacement in an amino acid sequence that changes a given amino acid to a different amino acid with similar biochemical, structural and/or chemical properties.

For example, amino acids may be sorted into six main classes on the basis of their structure and the general chemical characteristics of their side chains (R groups):
Aliphatic: Isoleucine (I), Leucine (L), Glycine (G), Alanine (A), Valine (V)
Hydroxyl or sulfur/selenium-containing: Serine(S), Cysteine (C), Threonine (T), Methionine (M)
Cyclic: Proline (P)
Aromatic: Phenylalanine (F), Tyrosine (Y), Tryptophan (W)
Basic: Histidine (H), Lysine (K), Arginine (R); and
Acidic and their amides: Aspartate (D), Glutamate (E), Asparagine (N), Glutamine (Q).

This means that an amino acid sequence having a defined % sequence identity of a reference amino acid sequence is preferably obtained by one or more conservative amino acid replacements of one or more amino acid residues in the reference amino acid sequence with a respective amino acid from the same R group listed above as the given amino acid residue.

In an embodiment, the VHH antibody has an amino acid sequence as defined in SEQ ID NO: 30. Such a VHH antibody is denoted KB_B01 herein.

In another embodiment, the VHH antibody has an amino acid sequence as defined in SEQ ID NO: 31. Such a VHH antibody is denoted KB_B02 herein.

In a further embodiment, the VHH antibody has an amino acid sequence as defined in SEQ ID NO: 32. Such a VHH antibody is denoted KB_B03 herein.

In yet another embodiment, the VHH antibody has an amino acid sequence as defined in SEQ ID NO: 33. Such a VHH antibody is denoted KB_B04 herein.

In another embodiment, the VHH antibody has an amino acid sequence as defined in SEQ ID NO: 34. Such a VHH antibody is denoted KB_B05 herein.

In a further embodiment, the VHH antibody has an amino acid sequence as defined in SEQ ID NO: 35. Such a VHH antibody is denoted KB_B06 herein.

In yet another embodiment, the VHH antibody has an amino acid sequence as defined in SEQ ID NO: 36. Such a VHH antibody is denoted KB_B07 herein.

In another embodiment, the VHH antibody has an amino acid sequence as defined in SEQ ID NO: 37. Such a VHH antibody is denoted KB_B08 herein.

In an embodiment, the VHH antibody has an amino acid sequence selected from the group consisting of SEQ ID NO: 30 to 37, preferably selected from the group consisting of SEQ ID NO: 30 to 36.

Another aspect of the invention relates to a VHH antibody binding specifically to a TfR1. The VHH antibody comprises a CDR1 consisting of the amino acid sequence DSAFX₂₈MNT as defined in SEQ ID NO: 38, wherein X₂₈ is S or N. The VHH antibody also comprises a CDR2 consisting of the amino acid sequence IVSDDNT as defined in SEQ ID NO: 39. The VHH antibody further comprises a CDR3 consisting of the amino acid sequence KGDVV as defined in SEQ ID NO: 40.

In a particular embodiment, the CDR1 consists of the amino acid sequence DSAFSMNT as defined in SEQ ID NO: 41, the CDR2 consists of the amino acid sequence IVSDDNT as defined in SEQ ID NO: 39 and the CDR3 consists of the amino acid sequence KGDVV as defined in SEQ ID NO: 40. A VHH antibody having CDR regions according to this particular embodiment is denoted KB_B09 herein.

In another particular embodiment, the CDR1 consists of the amino acid sequence DSAFNMNT as defined in SEQ ID NO: 42, the CDR2 consists of the amino acid sequence IVSDDNT as defined in SEQ ID NO: 39 and the CDR3 consists of the amino acid sequence KGDVV as defined in SEQ ID NO: 40. A VHH antibody having CDR regions according to this particular embodiment is denoted KB_B10 herein.

In an embodiment, the VHH antibody is of a formula: framework region 1 (FR1)-CDR1-FR2-CDR2-FR3-CDR3-FR4.

In an embodiment, the FR1 has an amino acid sequence QVQLQESGGGLVQVGGSLRLSCAAS as defined in SEQ ID NO: 43.

In an embodiment, the FR2 has an amino acid sequence MYWYRQAPGKX₂₉REFVAX₃₀ as defined in SEQ ID NO: 44, wherein X₂₉ is Q or S, and X₃₀ is Y or W.

In an embodiment, the FR3 has an amino acid sequence X₃₁YADSVKGRFTISRDNAKNTVYLQMNX₃₂LKPEDTAX₃₃YYC as defined in SEQ ID NO: 45, wherein X₃₁ is R or Q, X₃₂ is S or N, and X₃₃ is V or G.

In an embodiment, the FR4 has an amino acid sequence WGQGTQVTVSS as defined in SEQ ID NO: 29.

In a particular embodiment, the VHH antibody has amino acid sequences of FR1, FR2, FR3 and FR4 as defined in SEQ ID NO: 43, 44, 45 and 29.

The present invention also encompasses that the VHH antibody has an amino acid sequence of FR1 comprising or consisting of SEQ ID NO: 43, or a variant thereof having at least 88% sequence identity to SEQ ID NO: 43, preferably at least 92% sequence identity, and more preferably at least 96% sequence identity.

The present invention also encompasses that the VHH antibody has an amino acid sequence of FR2 comprising or consisting of SEQ ID NO: 44, or a variant thereof having at least 82% sequence identity to SEQ ID NO: 44, preferably at least 88% sequence identity, and more preferably at least 94% sequence identity.

The present invention also encompasses that the VHH antibody has an amino acid sequence of FR3 comprising or consisting of SEQ ID NO: 45, or a variant thereof having at least 92% sequence identity to SEQ ID NO: 45, preferably at least 94% sequence identity, and more preferably at least 97% sequence identity.

The present invention also encompasses that the VHH antibody has an amino acid sequence of FR4 comprising or consisting of SEQ ID NO: 29, or a variant thereof having at least 72% sequence identity to SEQ ID NO: 29, preferably at least 81% sequence identity, and more preferably at least 90% sequence identity.

In an embodiment, the VHH antibody has an amino acid sequence as defined in SEQ ID NO: 46. Such a VHH antibody is denoted KB_B09 herein.

In another embodiment, the VHH antibody has an amino acid sequence as defined in SEQ ID NO: 47. Such a VHH antibody is denoted KB_B10 herein.

In an embodiment, the VHH antibody has an amino acid sequence selected from the group consisting of SEQ ID NO: 46 to 47.

In another embodiment, the VHH antibody has an amino acid sequence selected from the group consisting of SEQ ID NO: 30-37, 46-47, preferably selected from the group consisting of SEQ ID NO: 30-36, 46-47.

FIG. 6 shows a sequence alignment of closely related VHH antibodies KB_B01 to KB_B10.

VHH antibodies of the present invention bind specifically to hTfR1.

The VHH antibodies bind specifically to hTfR1 both as monomeric VHH antibodies, see Table 2, but also in a fusion molecule linked to another molecule, see Tables 3, 5 and 7. In the latter case, the binding to hTfR1 by the VHH antibodies in a fusion molecule is not affected whether the other molecule is linked to the N-terminus of the VHH antibody or to the C-terminus of the VHH antibody, see Table 7. Furthermore, the binding of the VHH antibodies of the invention to hTfR1 is not greatly affected by the presence or absence of transferrin as shown in Table 4. This is in clear contrast to a benchmark VHH antibody BV, the affinity of which to hTfR1 was dramatically changed in the presence of 250 nM transferrin versus absence of transferrin as shown in Table 4.

By “bind specifically to” and similar expressions it is meant that the molecule in question, such as an VHH antibody, specifically binds to the target antigen without any significant binding to other molecules. The specificity of an antibody can be determined based on affinity and/or avidity. The affinity, represented by the equilibrium dissociation constant of an antigen with the antibody (K_{D}) is a measure for the binding strength between an antigenic determinant, i.e., epitope, and an antigen-binding site on the antibody. The lower the value of K_{D}, the stronger the binding strength between the antigenic determinant and the antibody. Alternatively, the affinity can also be expressed as the equilibrium association constant (K_{A}), which is 1/K_{D}. As will be clear to the skilled person, affinity can be determined in a manner known per se, depending on the specific antigen of interest.

Typically, antibodies will bind to their antigen with an equilibrium dissociation constant (K_{D}) of 10⁻⁵ to 10⁻¹² moles/liter (M) or less, and preferably 10⁻⁷ to 10⁻¹² M or less and more preferably 10⁻⁸ to 10⁻¹² M, i.e., with an affinity constant (K_{A}) of 10⁵ to 10¹² M⁻¹ or more, and preferably 10⁷ to 10¹² M⁻¹ or more and more preferably 10⁸ to 10¹² M⁻¹. Generally, any Ko value greater than 10⁻⁴ M (or any K_{A} value lower than 10⁴ M⁻¹) is considered to indicate non-specific binding.

Specific binding of an antibody to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, surface plasmon resonance (SPR), biolayer interferometry (BLI) and different variants thereof known per se in the art.

The affinity of VHH antibodies to TfR1 should be tailored to be within a specific range in order to achieve an efficient BBB crossing and a high relative uptake in the brain. Generally, if the affinity of antibodies to TfR1 is too low, such as a K_{D} larger than about 1 μM the antibodies have too low affinity for efficient binding to TfR1 and uptake by the endothelial cells of the BBB. This means that a large portion of the antibodies will remain in the peripheral blood system. Correspondingly, if the antibodies have too high affinity, such as K_{D} much lower than 1 nM (<<1 nM), the antibodies are endocytosed but are instead subject to lysosomal degradation in the endothelial cells of the BBB. This means that the VHH antibodies, for optimal receptor-mediated endocytosis, i.e., receptor-mediated cellular uptake, and transcytosis, i.e., transport across the interior of a cell, should have an affinity (K_{D}) in the low nM to the nM range (Bien-Ly N, et al., Transferrin receptor (TfR) trafficking determines brain uptake of TfR antibody affinity variants. J Exp Med. 211 (2): 233-244 (2014), WO 2016/081643).

As is shown in Table 2, the VHH antibody KB_B01 of the invention has, in monomeric form, an affinity (K_{D}) of 3.3 nM, i.e., in the nM range. Furthermore, the affinity still remains in the desired low nM to nM range when included in a VHH-containing fusion protein (see FIG. 2), such as a dimeric-VHH Fc fusion protein with a K_{D} of 0.18 nM, see Table 3, or a fusion with a scFv with a K_{D} of 0.85-1.84 nM, see Table 7. Also, the other VHH antibodies of the invention have affinities to hTfR1 in the sub-nM range when tested as dimeric Fc fusions and are thereby suitable for BBB-crossing by receptor-mediated endocytosis and transcytosis, see Table 5 with K_{D} ranging from below 0.0031 to 0.451 nM as dimeric-VHH Fc fusion proteins when binding to hTfR1 and with a 5 to 6000 higher Ko when binding to mTfR.

In an embodiment, the VHH antibodies of the invention bind specifically to human TfR1 (hTfR1).

In an embodiment, the VHH antibodies of the invention bind specifically to mTfR1. In a particular embodiment, the VHH antibodies of the invention bind not only specifically to hTfR1 but also to mTfR1, see Table 5.

In an embodiment, the VHH antibodies of the invention bind specifically to cTfR1. In a particular embodiment, the VHH antibodies of the invention also bind specifically to cTfR1 in addition to hTfR1 and mTfR, see Table 3.

Hence, in a preferred embodiment, the VHH antibodies exhibit a broad species cross-reactivity, i.e., bind with desired affinity to not only hTfR1 but also mTfR1 and cTfR1. This characteristic of the VHH antibodies is highly desirable from a translational point of view when predicting both pharmacokinetics and pharmacodynamics from mouse or monkey to human.

In an embodiment, the VHH antibodies of the invention bind specifically to mTfR1 with an affinity (K_{D}) selected within a range of from 0.1 to 150 nM, preferably within a range of from 0.1 to 100 nM. In a particular embodiment, the VHH antibodies of the invention, in monovalent form, bind specifically to mTfR1 with Ko selected within a range of from 1 to 150 nM, preferably within a range of from 1 to 100 nM, and more preferably within a range of from 1 to 50 nM.

In an embodiment, the VHH antibodies of the invention in monovalent or monomeric form bind specifically to hTfR1 with an affinity (K_{D}) selected within a range of from 0.01 to 150 nM, preferably within a range of from 0.01 to 100 nM.

Structurally, the TfR1 is a dimeric transmembrane glycoprotein with a large ectodomain (residues 90-760), an intramembranous region (residues 62-89) and the remaining 61 residues in the cytoplasm. The ectodomain has three domains, the helical domain (residues 606-760), the protease-like domain (residues 121-183, 384-605) and the apical domain (residues 184-383). The helical domain is responsible for receptor dimerization. Transferrin binds to the helical and protease-like domains.

The VHH antibodies of the invention bind to a similar epitope on hTfR1 as shown in Table 6 by being able to block binding of the other VHH antibodies of the invention to hTfR1. The VHH antibodies do not prevent human or mouse transferrin from binding to hTfR1 as shown in Table 6. Hence, the VHH antibodies of the invention do not interfere with the transferrin-binding for iron uptake by cells using the TfR1.

In an embodiment, the VHH antibodies of the invention are camelid VHH antibodies.

In another embodiment, the VHH antibodies of the invention are humanized VHH antibodies. For instance, the CDR regions of the VHH antibodies may be grafted onto a human backbone. Soler et al., Effect of Humanizing Mutations on the Stability of the Llama Single-Domain Variable Region, Biomolecules 11 (2): 163 (2021) identified several amino acid positions and the N-terminal Gln as hot sports for converting camelid VHHs to human consensus germ-line sequence.

Here below, sequence alignments are presented between the framework regions of KB_B01, a reference human VH (sVH), a universal VHH (uVHH) and a humanized VHH sequence (hVHH) as disclosed in the above-mentioned Biomolecules article.

QVQLQESGGGLVQAGGSLRLSCAAS KB_B01 FR1
----VQ-------P----------- sVH FR1
----V--------P--------T-- uVHH FR1
----V--------P----------- hVHH FR1
MRWYRQAPGKQREWVAG KB_B01 FR2
-S-V------GL---SP sVH FR2
L---------E--A--A uVHH FR2
L---------GL-A--A hVHH FR2
NYADSVKGRFTISRDNAEGTVYLQMDNLKPEDTAVYYC KB_B01 FR3
Y---------------SKN-L----NT-RA-------- sVH FR3
Y----------------KN--T---N--------I--- uVHH FR3
Y---------------SKN-L----NS-RA-------- hVHH FR2
WGQGTQVTVSS KB_B01 FR4
-----M----- sVH FR4
----------- UVHH FR4
-----L----- sVH FR4

The VHH antibodies of the invention can be humanized by modifications, e.g., amino acid substations, in FR1, FR2, FR3 and/or FR4. Amino acid positions in FR1-FR4 referred to herein are to the amino acid positions in KB_B01 (SEQ ID NO: 30) as shown in FIG. 6.

As an example, humanized positions in FR1 could be selected from E1 or Q1; V5; Q6 or E6; S11 or L11; P14; and/or R19. In an embodiment, any one of these humanized positions in FR1 are used for the humanized FR1 or any combination of two up to all of these positions are humanized. As an example, a humanized FR1 could be based on SEQ ID NO: 26 or 77 but have Q1 replaced by E, and Q5 replaced by V, or two or all of these amino acid replacements.

Humanized positions in FR2 could be selected from L34 or M34 (first amino acid position in FR2), S35 or G35 (second amino acid position in FR2), V37 or F37 (fourth amino acid position in FR2), Q43 or K43 (tenth amino acid position in FR2), G44 (eleventh amino acid position in FR2), L45 (twelfth amino acid position in FR2), E46 (13^{th} amino acid position in FR2), W47 or A47 (14^{th} amino acid position in FR2), S49 (16^{th} amino acid position in FR2) and/or P50, A50, V50 or G50 (17^{th} amino acid position FR2). In an embodiment, any one of these humanized positions in FR2 are used for the humanized FR2 or any combination of two up to all of these positions are humanized. As illustrative example, the humanized positions in FR2 could be V37, G44, L45 and W47 or F37, G44, L45 and A47.

Humanized positions in FR3 could be selected from Y58 (1^{st} amino position in FR3), S74 (17^{th} amino acid position in FR3), K75 (18^{th} amino acid position in FR3), N76 (19^{th} amino acid position in FR3), L78 or 178 (21^{st} amino acid position in FR3), N84, S84 or T84 (27^{th} amino acid position in FR3), R86 (29^{th} amino acid position in FR3), A87 (30^{th} amino acid position in FR3), A96 (39^{th} amino acid position in FR3) and/or R97 or A97 (40^{th} amino acid position in FR3). In an embodiment, any one of these humanized positions in FR3 are used for the humanized FR3 or any combination of two up to all of these positions are humanized.

Humanized positions in FR4 could be L106 or M106 (sixth amino acid position in FR 4).

I78 also has stabilizing effect for VHHs. Hence, Ile at position 78 stabilizes the VHH.

In an embodiment, the VHH antibodies of the invention bind to protein A. In a particular embodiment, the VHH antibodies of the invention bind to protein A based resins, such as protein A chromatography resins, such as MabSelect PrismA™ resin.

Protein A is a 42 kDa surface protein originally found in the cell wall of the bacteria Staphylococcus aureus. It is encoded by the spa gene. It has found use in biochemical research because of its ability to bind immunoglobulins. It is composed of five homologous Ig-binding domains each folded into a three-helix bundle. Each domain is able to bind proteins from many mammalian species, most notably IgGs. It binds the heavy chain within the Fc region of most immunoglobulins and also within the Fab region in the case of the human VH3 family. Protein A generally does not bind or merely bind weakly to camelid VHH antibodies.

Henry K A, et al., A Rational Engineering Strategy for Designing Protein A-Binding Camelid Single-Domain Antibodies, PLoS One 11 (9): e0163113 (2016) discloses how to make non-protein A binding VHH antibodies capable of binding to protein A, see Table 5.

In more detail, amino acid residue 15 should be G or D, amino acid residue 17 should be S or A, amino acid residue 19 should be R. The FR1 sequence of KB_B01 is a protein A binding FR1 region as indicated below.

QVQLQESGGGLVQAGGSLRLSCAAS KB_B01 FR1
----------S-------------- Protein A binding FR1

Furthermore, amino acid residue 59 should be Y, amino acid residue 64 should be K or E, amino acid residue 65 should be G, amino acid residue 66 should be R, amino acid residue 68 should be T or A, amino acid residue 70 should be S, amino acid residue 75 should be A, E, K, Q or R, amino acid residue 81 should be Q, amino acid residue 83 should be N, and amino acid residue 84 should be S, N or G. The FR3 region of KB_B01 is protein A binding by replacing the amino acid residue 83 from D to N. However, in an optional embodiment, the N at amino acid residue 84 could be replaced by S.

NYADSVKGRFTISRDNAEGTVYLQMDNLKPEDTAVYYC KB_B01 FR3
E----------------KN------NS----------- Protein A binding FR3

In an embodiment, the FR1 region has R at amino acid residue 19 with R and the FR3 region has N at amino acid position 83 and S at amino acid position 84.

In an embodiment, the VHH antibody of the invention is an isolated VHH antibody.

The term “isolated” when used in connection with VHH antibodies, such as in the expression “isolated VHH antibody” and similar expressions, means the VHH antibody has been purified removed from its original environment. An isolated VHH antibody, as used herein, is intended to refer to a VHH antibody that is substantially free of other antibodies having different antigenic specificities, e.g., an isolated VHH antibody that specifically binds TfR1, in particular human TfR1 (hTfR1), is substantially free of antibodies that specifically bind antigens other than TfR1. An isolated VHH antibody that specifically binds hTfR1 may, however, have cross-reactivity to other antigens, such as TfR1 molecules from other species, such as cTfR1 and mTfR1. Moreover, an isolated VHH antibody may be substantially free of other cellular material and/or chemicals. For example, the isolated VHH antibody may be purified to greater than 95% or 99% purity as determined by, for example, electrophoretic, e.g., sodium dodecyl sulfate-polyacrylamide gel electrophoresis (SDS-PAGE), isoelectric focusing (IEF), capillary electrophoresis, or chromatographic methods, e.g., ion exchange or reverse-phase high-performance liquid chromatography (HPLC). The skilled person will appreciated that isolated VHH antibodies are referred to herein even though the word “isolated” is not explicitly mentioned each time the term “VHH antibodies” and the like are used.

A further aspect of the embodiments includes a nucleic acid molecule encoding a VHH antibody according to the embodiments or a fusion molecule according to the embodiments, see further below. Nucleic acid molecule as used herein includes polynucleotide, oligonucleotide, and nucleic acid sequence, and generally means a polymer of DNA or RNA, which may be single-stranded or double-stranded, which may contain natural, non-natural or altered nucleotides, and which may contain a natural, non-natural or altered internucleotide linkage, such as a phosphoroamidate linkage or a phosphorothioate linkage, instead of the phosphodiester found between the nucleotides of an unmodified oligonucleotide. The term “nucleic acid molecule” also include complementary DNA (cDNA) and messenger RNA (mRNA).

In an embodiment, the nucleic acid molecule is an isolated nucleic acid molecule encoding a VHH antibody according to the embodiments or a fusion molecule according to the embodiments.

The nucleic acid molecules may encode a single VHH antibody according to the embodiments, multiple copies of a single VVH antibody according to the embodiments, or one or more copies of different VHH antibodies according to the embodiments.

The nucleic acid molecule may also encode other molecules than the VHH antibody of the embodiments, for instance the VHH antibody genetically fused to another molecule, i.e., in the form of a VHH-containing fusion protein as further described herein.

Another aspect of the embodiments relates to a vector comprising a nucleic acid molecule according to the embodiments.

The vector is preferably an expression vector, i.e., a vector comprising at least one nucleic acid molecule comprising coding sequences that can be expressed, such as transcribed and translated, in a host cell comprising the expression vector. The expression vector therefore comprises a nucleic acid molecule according to the embodiments operative linked to a promoter.

Operatively linked as used herein means that the nucleic acid molecule is in a correct functional location and/or orientation in relation to the promoter to enable expression of the nucleic acid molecule in the host cell, i.e., the promoter (constitutively or inducibly) controls transcription of the nucleic acid molecule.

The expression vector is in an embodiment selected among DNA molecules, RNA molecules, plasmids, episomal plasmids and virus vectors. In such an embodiment, the expression vector comprises, in addition to the nucleic acid sequence encoding the VHH antibody, control sequences needed to produce the VHH antibody in the host cell. For instance, the nucleic acid sequence encoding the VHH antibody is under transcriptional control of a promoter sequence comprised in the expression vector. A promoter is a sequence of DNA, to which proteins bind that initiate transcription of an RNA molecule from the DNA (gene) downstream of it. The promoter is preferably selected based on the particular host cell, in which the VHH antibody is to be expressed, such as the T5, T7, lac, BL21 for expression of the VHH antibody in bacterial cells, GAL1, MET25, CUP1, LAC4, ADH2, SUC2, GAPDH for expression of the VHH antibody in yeast and EF1α, CMV or CAG promoter for expression in mammalian cells, such as human cells. The promoter could be a constitutive promoter or an inducible promoter. A constitutive promoter, also referred to as constitutively active promoter, is active in all circumstances in the host cell. An inducible promoter, also referred to as inducibly active promoter, is regulated and becomes active in the host cell only in response to specific stimuli, such as a chemically inducible promoter, a temperature inducible promoter, or a light inducible promoter. The expression vector may optionally comprise other control sequences, such as an enhancer. An enhancer is a short region of DNA that can be bound by activators to increase the likelihood that transcription of a particular gene will occur. An optional signal peptide could be provided at the N-terminus or the C-terminus of the polypeptide encoded by the expression vector.

A further aspect of the embodiments relates to a host cell comprising a nucleic acid molecule or an expression vector according to the embodiments.

The nucleic acid molecule or expression vector can then be transcribed in the host cell to produce the VHH antibody in the cell.

In an embodiment, the cell is selected from the group consisting of a bacterial cell, a yeast cell, and a mammalian cell.

Yet another aspect of the embodiments relates to a VHH antibody according to the invention linked to at least one molecule. Hence, this aspect of the invention relates to a fusion molecule or protein between the VHH antibody and the at least one other molecule. The fusion molecule or protein is preferably in the form a genetic fusion between the VHH antibody and the at least one molecule. In such a case, the above-mentioned nucleic acid molecule encodes not only the VHH antibody but also at least one other molecule. For instance, the nucleic acid molecule could include from a 5′ end to a 3′ end a nucleic acid sequence encoding the VHH molecule and a nucleic acid sequence encoding the at least one other molecule or a nucleic acid sequence encoding the at least one other molecule and the nucleic acid sequence encoding the VHH molecule. Hence, the VHH molecule can be covalently linked to the at least one other molecule at its N-terminus or its C-terminus, see FIG. 2.

It is further possible to produce a fusion molecule comprising more than one molecule in addition to the VHH antibody. For instance, multiple different molecules or multiple copies of a single molecule could be connected or attached to the N-terminus of the VHH antibody, to the C-terminus of the VHH antibody, or at least one molecule is connected or attached to the N-terminus of the VHH antibody and at least one molecule is connected or attached to the C-terminus of the VHH molecule.

In an embodiment, the fusion molecule comprises the at least one other molecule connected or attached to the C-terminus of the VHH antibody.

As mentioned above, the VHH antibody is preferably linked to the at least one other molecule by being genetically fused to the to the at least one other molecule. In such an embodiment, the nucleic acid molecule encodes the VHH antibody and the at least one other molecule as a VHH-containing fusion protein. This means that the nucleic acid molecule comprises a nucleic acid sequence encoding the VHH antibody connected or linked to a nucleic acid sequence encoding the at least one other molecule and where these nucleic acid sequences are under transcriptional control of a same promoter.

The VHH antibody can, however, be linked, connected, attached or conjugated to the at least one molecule in other ways than genetically fused, such as by chemically connecting the VHH antibody to the at least one molecule. For instance, the VHH antibody could be covalently connected to the at least one molecule by a reaction between a maleimide with amines or thiols, often referred to as thiol-maleimide or amine-maleimide click chemistry. In such an embodiment, an additional amino acid residue, such as cysteine, arginine, or lysine, may be added to the N-terminus and/or C-terminus of the VHH antibody to enable such a reaction with maleimide.

It is also possible to enzymatically link the VHH antibody and the at least one molecule, such as by the transglutaminase (TGase) enzyme, which catalyzes the formation of an isopeptide bond between γ-carboxamide groups (—(C═O)NH₂) of glutamine residue side chains and the ε-amino groups (—NH₂) of lysine residue side chains with subsequent release of ammonia (NH₃). In such an embodiment, an additional amino acid residue, such as glutamine or lysine, may be added to the N-terminus and/or C-terminus of the VHH antibody to enable such an enzymatic reaction.

In an embodiment, the VHH antibody according to the invention is covalently linked to at least one molecule.

The VHH antibody could be linked, such as fused, directly to the at least one molecule. Alternatively, the VHH antibody is covalently linked to the at least one molecule though a linker. Various such linkers, in particular peptide linkers, could be used according to the embodiments including, but not limited, to Gₙ linkers, wherein n is an integer equal to or larger than 1 and typically equal to or smaller than 10, Sₘ likers, wherein m is an integer equal to or larger than 1 and typically equal to or smaller than 10, A_{q} linkers, wherein q is an integer equal to or larger than 1 and typically equal to or smaller than 10, various GS-linkers or GA-linkers, i.e., combinations of one or more G with one or more S or one or more A, such as (GₙSₘ)ₚ or (SₘGₙ)ₚ or (GₙA_{q})ₚ or (A_{q}Gₙ)ₚ, wherein p is an integer equal to or larger than 1 and typically equal to or smaller than 10. For instance, linkers such as G₄A, G₄S, G₃S, and combinations thereof, such as G₄A-G₄A-G₄S or G₄S-G₃S, could be used. Other commonly used peptide linkers are disclosed in Table 1 of Vishnu Priyanka Reddy Chichil, et al., Linkers in the structural biology of protein-protein interactions, Protein Science 22 (″): 153-167 (2013), the linkers listed in Table 1 on pages 156-157 are hereby incorporated by reference as illustrative, but non-limiting, examples of peptide linkers that could be used to covalently interconnect the VHH antibody and the at least one molecule.

The fusion molecule or the VHH antibody of the invention may comprise one or multiple tags, including, but not limited, to affinity purification tags, such as a His tag, a C-tag, a Q-tag, and/or a myc tag. Such a tag may then be present at the N-terminus and/or C-terminus of the fusion molecule or the VHH antibody. Addition of such a tag to enable purification may also be accompanied by a protease site such as, but not limited to, a TEV site for efficient subsequent removal of the purification tag from the VHH antibody or the fusion molecule.

The fusion molecule could also comprise multiple VHH antibodies in addition to at least one molecule as shown in FIG. 2.

In an embodiment, the fusion molecule is a monovalent fusion molecule with regard to the VHH antibody, i.e., preferably only comprises a single VHH antibody in addition to the at last one molecule. There are in vitro and in vivo evidence that the monovalent binding mode facilitates transcellular transport, whereas a bivalent binding mode leads to lysosome sorting (Niewoehner et al., Increased brain penetration and potency of a therapeutic antibody using a monovalent molecular shuttle, Neuron. 81 (1): 49-60 (2014)).

The at least one molecule may be any molecule such as a medicament or drug, a diagnostic agent, an imaging agent, a tracer, a half-life extending agent, etc. Examples of such molecules include, without limitation, an antibiotic, antiviral, immunomodulator, antineoplastic, anti-inflammatory, adjuvant, peptides, polypeptides and proteins, such as an enzyme, hormone, neurotrophic factor, neuropeptide, cytokine, apolipoprotein, growth factor, antigen, antibody or part of an antibody, adjuvant, etc., nucleic acids, such as RNA or DNA including e.g., coding genes, inhibitory nucleic acids, such as ribozymes, antisense, interfering nucleic acids, full genomes or portions thereof, plasmids, etc.

In an embodiment, the at least one molecule of the fusion molecule is selected from the group consisting of a therapeutic agent and an imaging agent.

Imaging agent as used herein refers to an agent or molecule that is used during an imaging process to visualize the imaging agent, such as when administered in a patient body, as taken up by cells. Examples of such imaging agents include agents or molecules comprising a radioactive atom, or isotope, such as a radiotracer of a position emission tomography (PET) tracer, such as a ¹⁸F containing PET tracer, a ¹¹C containing PET tracers, ⁶⁴Cu containing PET tracers, or single-photon emission computerized tomography (SPECT) tracers, such as ^{99m}Tc or ¹¹¹In containing SPECT tracers. Other examples of imaging agents include fluorescent probes, luminescent probes, metal complex containing probes, near infrared (NIR) fluorescent probes, etc.

Generally, there is an urgent need for diagnostic tools enabling early detection of diseases or medical conditions, in particular in the brain compartment. Such an early detection would facilitate efficient disease-modifying therapies, especially to evaluate treatment effects in preclinical and clinical trials of new drug candidates.

Specific targeting of disease-causing proteins, including, but not limited to, amyloid beta, tau and alpha-synuclein, or pseudomarkers of disease, such as inflammation, is difficult with classical PET ligands or tracers based on small molecules capable of cross the BBB. It would generally be preferred to use PET ligands or tracers based on specific binding molecules, such as antibodies. However, PET tracers based on monoclonal antibodies (mAbs) generally have too long circulation half-life to be of any use for PET ligands or tracers. This means that the radioactivity originating from radiolabeled mAbs in the blood circulation of the brain will interfere with the specific signal at the brain target site. Therefore, in order to be useful in PET applications, antibody-based PET ligands or tracers should have a fast blood clearance with high brain-to-blood ratio. Smaller antibody domains or fragments (average molecular weight equal to or below 60 kDa), such as VHH antibodies, have a faster systemic elimination as compared to mAbs due to excretion via the kidney. Hence, VHH antibodies, due to their smaller size as compared to mAbs, are suitable as PET ligands or tracers (Syvänen, et al., A bispecific Tribody PET radioligand for visualization of amyloid-beta protofibrils—a new concept for neuroimaging. Neuroimage 148:55-63. (2017); Vandesquille, et al., Chemically-defined camelid antibody bioconjugate for the magnetic resonance imaging of Alzheimer's disease. mAbs 9:1016-1027 (2017)).

In these embodiments, the fusion molecule can be used as diagnostic agent for diagnosis of various diseases or disorders.

For instance, the VHH antibody of the embodiments could be linked to another antibody, antigen-binding fragment or domain thereof, including another VHH antibody, which binds specifically to a disease specific marker. In such a case, the other antibody is preferably labeled to detect its binding to the disease specific marker once delivered, such as to the brain compartment by receptor-mediated transcytosis using the VHH antibody as BBB transporter.

Antigen-binding fragment or domain of an antibody as used herein can be selected from a group consisting of a single chain antibody, a Fv fragment, a scFv fragment, a Fab fragment, a F(ab′)₂ fragment, a Fab′ fragment, a Fd fragment, a single-domain antibody (sdAb), a scFv-Fc fragment, a di-scFv fragment and a group of two or more CDR regions.

For therapeutic purposes, the fusion molecule could comprise a half-life extending agent or group, also referred to as a stabilizing agent or group, in addition to the therapeutic agent. Such a half-life extending agent is then included to increase the half-life of the fusion molecule when administered to a patient. Any such half-life extending agent that can be linked to the VHH antibody but with no adverse biological effect could be used including, but not limited to, a Fc fragment of an IgG, serum proteins, such as human serum albumin (HSA), albumin-binding protein scaffolds, an IgG, various polyethylene glycol (PEG) molecules, or unstructured polypeptides, such as XTEN, or PASylation as a substitute for PEGylation.

The present invention also relates to a pharmaceutical composition comprising a fusion molecule as defined above, wherein the at least one molecule is a therapeutic agent. The pharmaceutical composition also comprises a pharmaceutically acceptable vehicle or excipient.

The pharmaceutically acceptable vehicle could be any pharmaceutically acceptable vehicle or carrier that is compatible with the other constituent(s) of the pharmaceutical composition. The pharmaceutically acceptable vehicle can be selected from the vehicles traditionally used according to each mode of administration. The pharmaceutical composition could be a solid pharmaceutical composition, such as a tablet, pill, powder, or granules, a semi solid pharmaceutical composition, such as a suppositories; or a liquid pharmaceutical composition, such as soft capsule or injection solution.

Pharmaceutically acceptable vehicles or excipients include, but are not limited to, diluents, such as lactose, dextrose, sucrose, mannitol, sorbitol, cellulose, and glycine; lubricants, such as silica, talc, stearic acid including salts thereof, and polyethylene glycol; binders, such as magnesium and aluminum silicate, starch paste, gelatin, tragacanth, methylcellulose, sodium carboxymethyl cellulose, and polyvinylpyrrolidone; disintegrants, such as starch, agar, alginic acid, and sodium alginate; absorbents; dyes; flavoring agents; sweeteners; polypropylene glycol; liquid vehicles, such as water, physiological saline solution, aqueous dextrose, glycerol, ethanol, and oil.

The embodiments also relate to a fusion molecule according to above for use as a medicament, wherein the at least one molecule is a therapeutic molecule.

In an embodiment, the therapeutic agent is capable of treating a CNS disease or disorder.

In a particular embodiment, the fusion molecule according to above is for use in treatment of a CNS disease or disorder. In such an embodiment, the at least one molecule is therapeutic agent capable of treating the CNS disease or disorder.

A further embodiment is directed towards a method of treating a CNS disease or disorder in a patient. The method comprises administering an effective amount of a fusion molecule according to above or a pharmaceutical composition according to above to the patient. In such an embodiment, the at least one molecule is therapeutic agent capable of treating the CNS disease or disorder.

Illustrative, but non-limiting, examples of CNS disease or disorders include Alzheimer's disease (AD), Bell's palsy, cerebral palsy, epilepsy, motor neuron diseases (MND), such as amyotrophic lateral sclerosis (ALS), progressive bulbar palsy (PBP), pseudobulbar palsy, progressive muscular atrophy (PMA), primary lateral sclerosis (PLS), spinal muscular atrophy (SMA) and monomelic amyotrophy (MMA), multiple sclerosis (MS), neurofibromatosis, Parkinson's disease (PD), lysosomal storage disease, neuronopathic lysosomal storage diseases, ischemic stroke, intracerebral hemorrhage, traumatic brain injury (TBI), vascular dementia, frontotemporal dementia, amyloidosis, tauopathy, Creutzfeldt-Jakob disease, neuroinflammation and neuropathic pain.

Malignant cells often overexpress TfR1 and this increased expression can be associated with poor prognosis in different types of cancer (Candelaria et al., Antibodies Targeting the Transferrin Receptor 1 (TfR1) as Direct Anti-cancer Agents. Front. Immunol. 12:607692 (2021)). TfR1 is overexpressed on many different types of cancer cells, often at levels several-fold higher than normal cells. In fact, TfR1 has been identified as a universal cancer marker. Increased expression of TfR1 correlates with advanced stage and/or poorer prognosis in a number of cancers, including solid cancers, such as esophageal squamous cell carcinoma, breast cancer, ovarian cancer, lung cancer, cervical cancer, bladder cancer, osteosarcoma, pancreatic cancers, cholangiocarcinoma, renal cell carcinoma, hepatocellular carcinoma, adrenal cortical carcinoma, glioblastoma multiforme (GBM) and cancers of the nervous system as well as hematopoietic malignancies, such as acute lymphoblastic leukemia (ALL), chronic lymphocytic leukemia (CLL), and non-Hodgkin lymphoma (NHL) (Candelaria et al., Antibodies Targeting the Transferrin Receptor 1 (TfR1) as Direct Anti-cancer Agents. Front. Immunol. 12:607692 (2021); Ramalho et al., Transferrin Receptor-Targeted Nanocarriers: Overcoming Barriers to Treat Glioblastoma. Pharmaceutics 14 (2): 279 (2022).

The elevated levels of TfR1 expression on malignant cells, together with its extracellular accessibility, ability to internalize, and central role in cancer cell pathology make this receptor an attractive target for antibody-mediated therapy. The TfR1 can be targeted by VHH antibodies of the invention for cancer therapy through the use of VHH antibodies conjugated to anti-cancer agents that are internalized by receptor-mediated endocytosis.

In another embodiment, the therapeutic agent is capable of treating cancer.

In a particular embodiment, the fusion molecule according to above is for use in treatment of cancer. In such an embodiment, the at least one molecule is therapeutic agent, immunotherapy agents and/or adjunctive therapy agents capable of treating cancer.

A further embodiment is directed towards a method of treating cancer in a patient. The method comprises administering an effective amount of a fusion molecule according to above or a pharmaceutical composition according to above to the patient. In such an embodiment, the at least one molecule is therapeutic agent capable of treating cancer.

The hTfR1 has also been a target to deliver therapeutic agents, including oligonucleotides, to muscle (Desjardins et al., Enhanced exon skipping and prolonged dystrophin restoration achieved by TfR1-targeted delivery of antisense oligonucleotide using FORCE™ conjugation in mdx mice, Nucleic Acids Research gkac641, 2022; Sugo et al., Development of antibody-siRNA conjugate targeted to cardiac and skeletal muscles, J Control Release 237:1-13 (2016)). Accordingly, the fusion molecule of the invention can be used to treat various muscular diseases and in particular muscular dystrophy.

The fusion molecule could also be used to treat muscular dystrophy, such as Duchenne muscular dystrophy (DMD), Becker muscular dystrophy (BMD), mytonic muscular dystrophy (DM), limb-girdle muscular dystrophy (LGMD), facioscapulohumeral muscular dystrophy (FSHD), congenital muscular dystrophy (CMD), distal muscular dystrophy (DD), oculopharyngeal muscular dystrophy (OPMD), or Emery-Dreifuss muscular dystrophy (EDMD), in particular DMD, FSHD or DM.

In a further embodiment, the therapeutic agent is capable of muscular dystrophy.

In a particular embodiment, the fusion molecule according to above is for use in treatment of muscular dystrophy. In such an embodiment, the at least one molecule is therapeutic agent capable of treating muscular dystrophy.

A further embodiment is directed towards a method of treating muscular dystrophy in a patient. The method comprises administering an effective amount of a fusion molecule according to above or a pharmaceutical composition according to above to the patient. In such an embodiment, the at least one molecule is therapeutic agent capable of treating muscular dystrophy.

As used herein, effective amount indicates an amount effective, at dosages and for periods of time necessary to achieve a desired result. Effective amounts may vary according to factors, such as the disease state, age, sex, weight of the patient. Treating or treatment as used herein and is well understood in the art, means an approach for obtaining beneficial or desired results, including clinical results. Beneficial or desired clinical results could include, for instance, alleviation or amelioration of one or more symptoms or conditions, diminishment of extent of disease, stabilized state of disease, i.e., prevent worsening, preventing spread of disease, delay or slowing of disease progression, amelioration or palliation of the disease state, diminishment of the reoccurrence of disease, and remission. Treating or treatment may also prolong survival as compared to expected survival if not receiving any treatment.

Preventing or prophylaxis as used herein and is well understood in the art, means an approach in which a risk of developing a disease or condition is reduced or prevented, including prolonging or delaying disease development. For instance, a patient predisposed to develop a disease, such as due to genetic or hereditary predisposition, could benefit for administration of the fusion molecule or the pharmaceutical composition to prevent, reduce the risk of, delaying and/or slowing development of the disease.

The patient is preferably a human patient. The embodiments may, however, also be applied in veterinary applications, i.e., non-human patients, such as non-human mammals including, for instance, primates, monkeys, apes, cattle, sheep, pigs, goats, horses, cats, dogs, mice, rats and guinea pigs.

The fusion molecule or the pharmaceutical composition according to the embodiments may be administered to the patient according to various routes including, for instance, parenteral route, such as injection by subcutaneous, intravenous, intraperitoneal or intramuscular route; oral route; rectal route; topical route; intranasal route; perlingual route; or intraocular route. In a particular embodiment, the administration route is a subcutaneous or intravenous administration.

### EXAMPLES

### Example I—Production of Proteins for Llama Immunization and Assays

The extracellular domains of hTfR1 (amino acids R121-F760, SEQ ID NO: 48), mTfR1 (S122-F763, SEQ ID NO: 49) and cTfR1 (R121-F760, SEQ ID NO: 50) were cloned into a pCDNA3.4-TOPO plasmid with the 5′ cloning sites: HindIII, KpnI, NheI and 3′ cloning sites: XhoI, PacI, using stop codons: TGA, TGA and a Kozak sequence (generated with Geneart tool (Thermo Fischer Scientific), optimized for expression in Cricetulus griseus. An N-terminal signal peptide (MDWLRNLLFLMAAAQSINA, SEQ ID NO: 51) followed by a single amino acid linker “A”, a Histidine tag (6×His, SEQ ID NO: 56) and an SG-linker (hTfR1, cTfR1) or a G-linker (mTfR1) were used for expression. Plasmids were ordered from Geneart (Thermo Fischer Scientific, Germany). Transient transfection of Chinese hamster ovarian (CHO) cells was performed according to standard procedures. Cultures were harvested on Day 6. Supernatants were filtered (0.45 μm syringe filter) before purification with immobilized metal ion chelate (IMAC) using a HisTrapExcel 5 mL column (Cytiva). Buffers used were as follows: equilibrium/wash (50 mM phosphate, 500 mM NaCl, pH 8), primary wash buffer (50 mM phosphate, 500 mM NaCl, 20 mM Imidazole, pH 8) and elution buffer (50 mM phosphate, 500 mM, NaCl 500 mM, Imidazole, pH 8). Eluted fractions were concentrated with an Amicon Ultra 10000 MWCO spin concentrator. Proteins were dialyzed overnight in PBS with 10% glycerol (Medicago, tablet, glycerol diluted from 80% stock solution) at 4° C. (cassette slide-a-lyzer 3 ml), 10 kDa cutoff (Pierce). PBS was set to pH 8. The protein concentrations were measured using absorbance at 280 nm.

### Example II—Generation of VHH Library that Bind the Human TfR1 and Selection

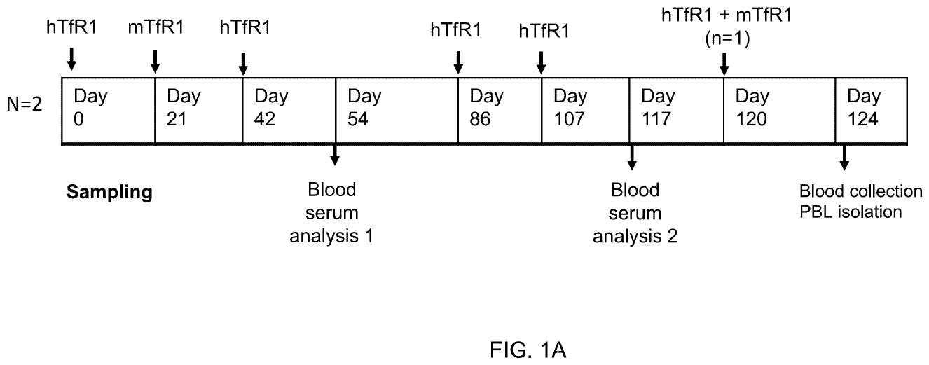

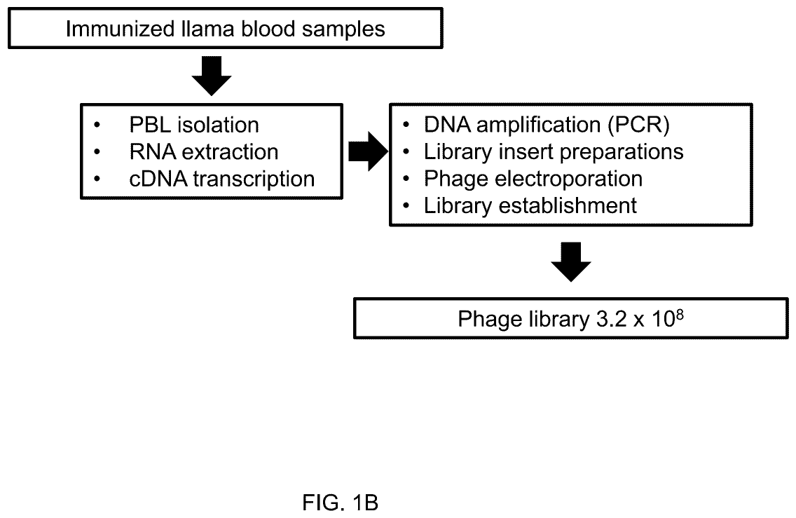

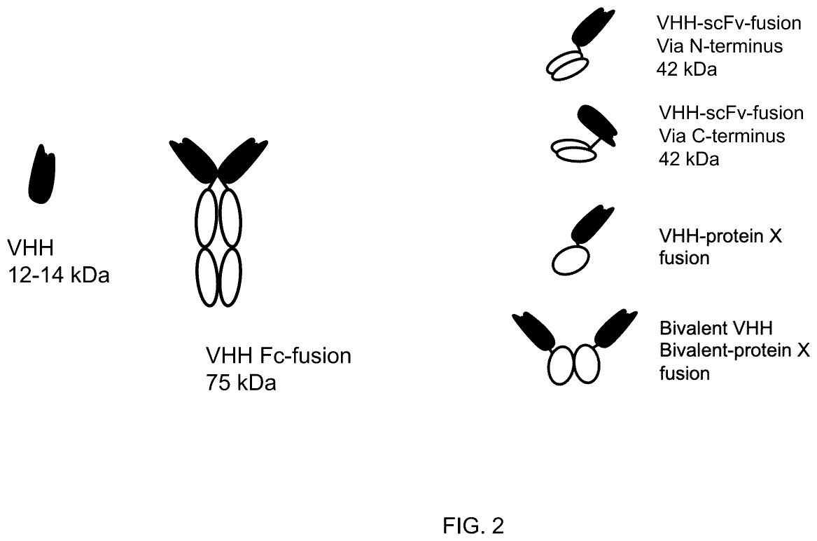

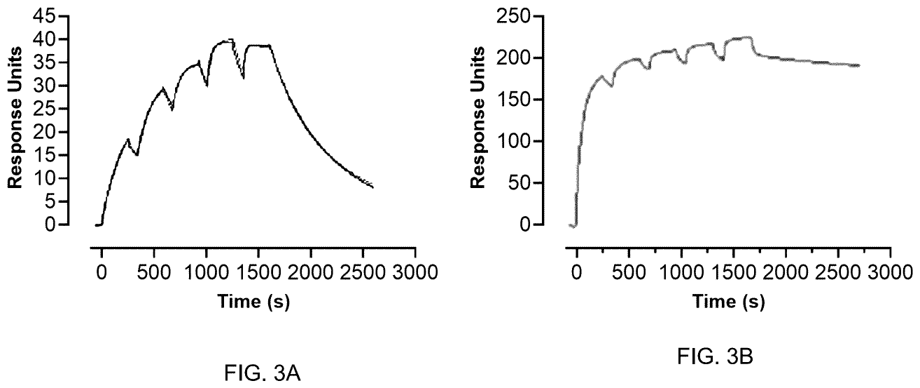

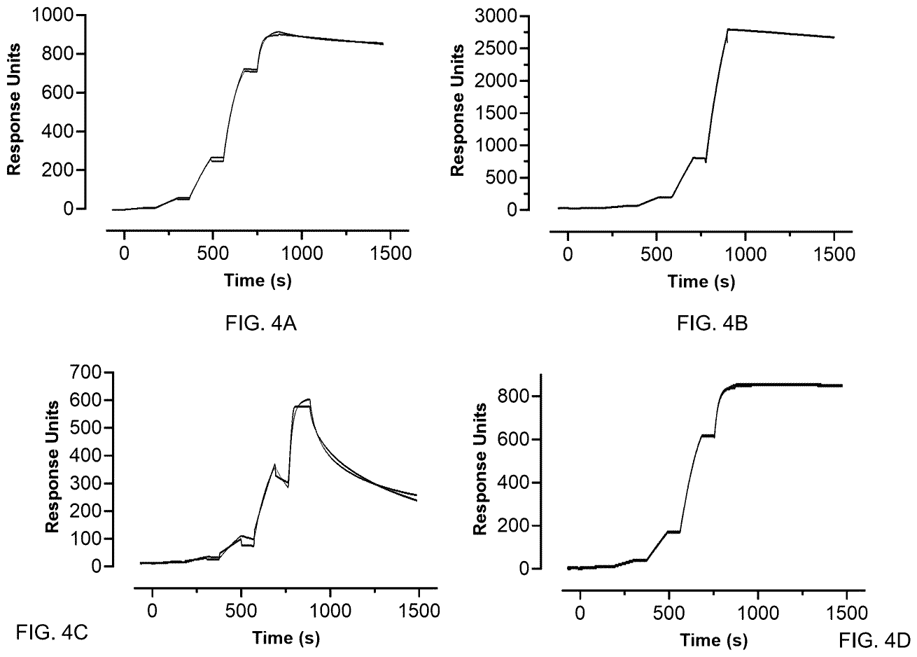

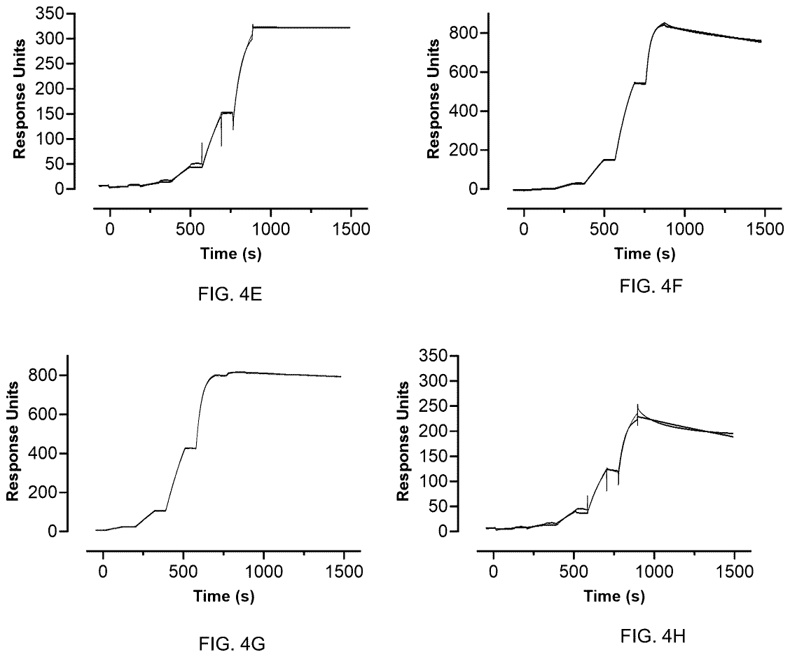

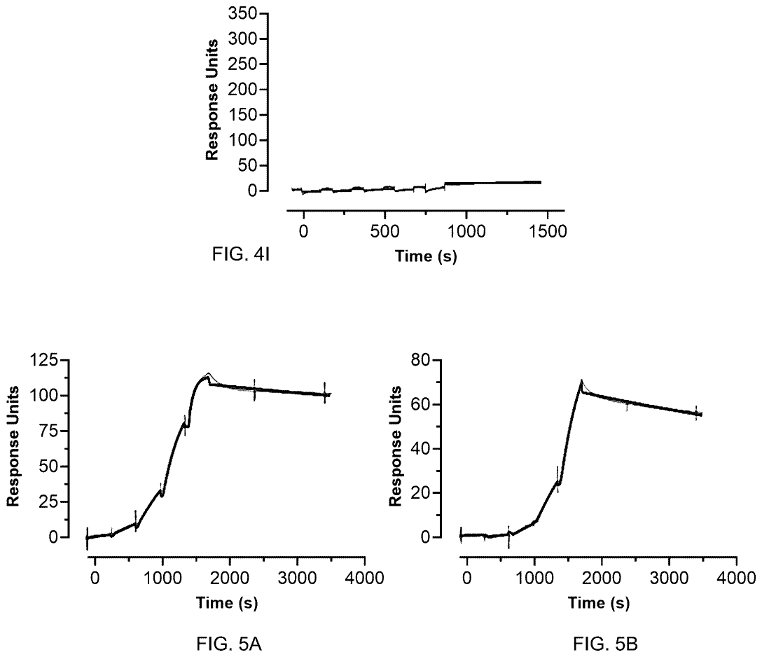

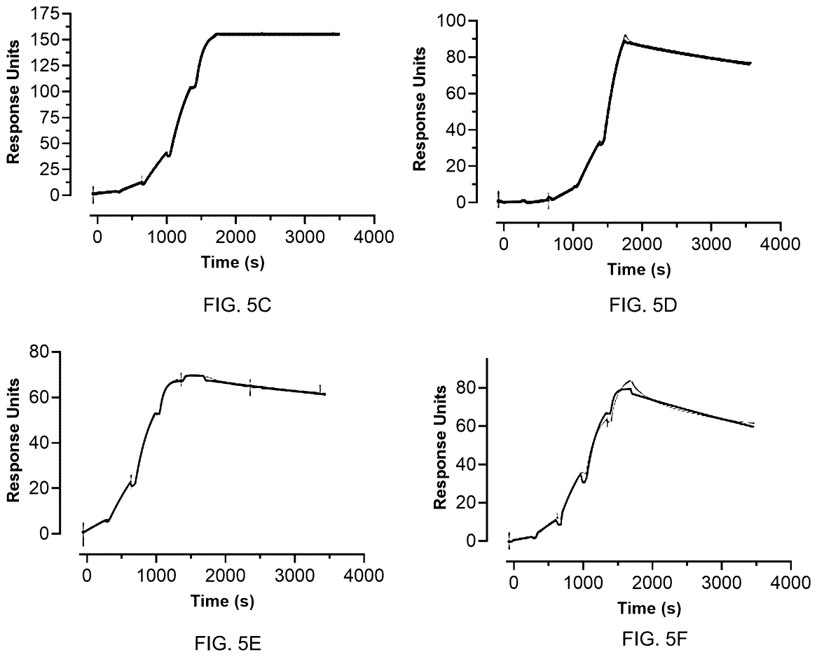

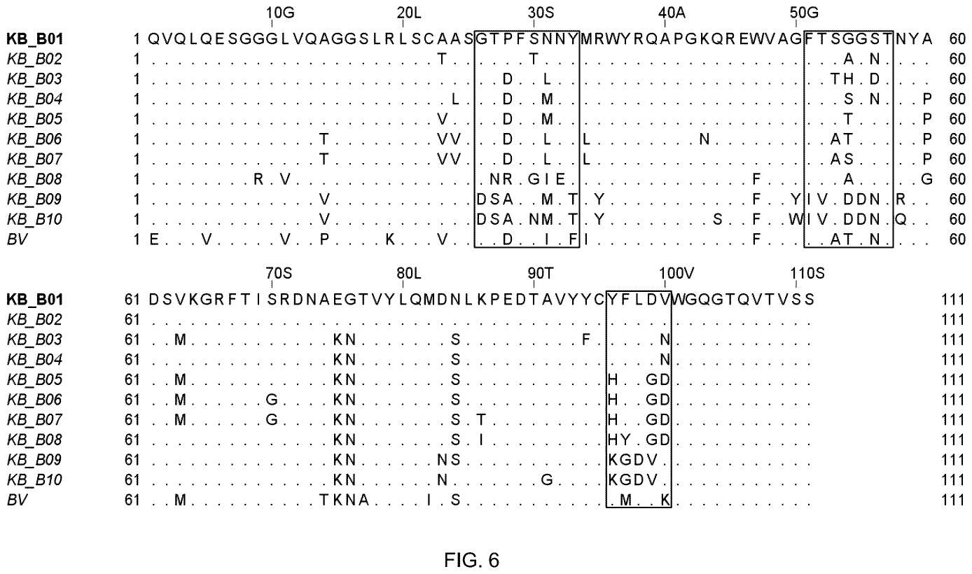

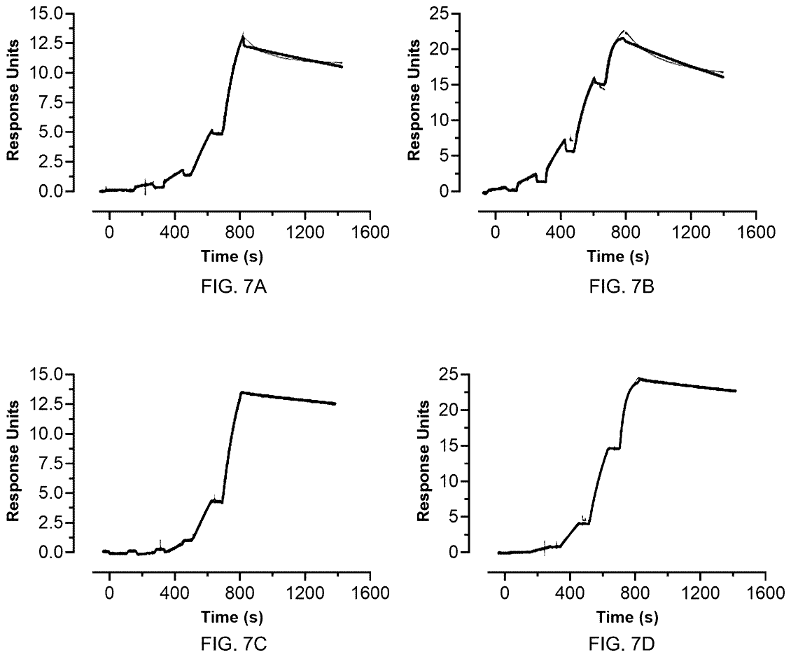

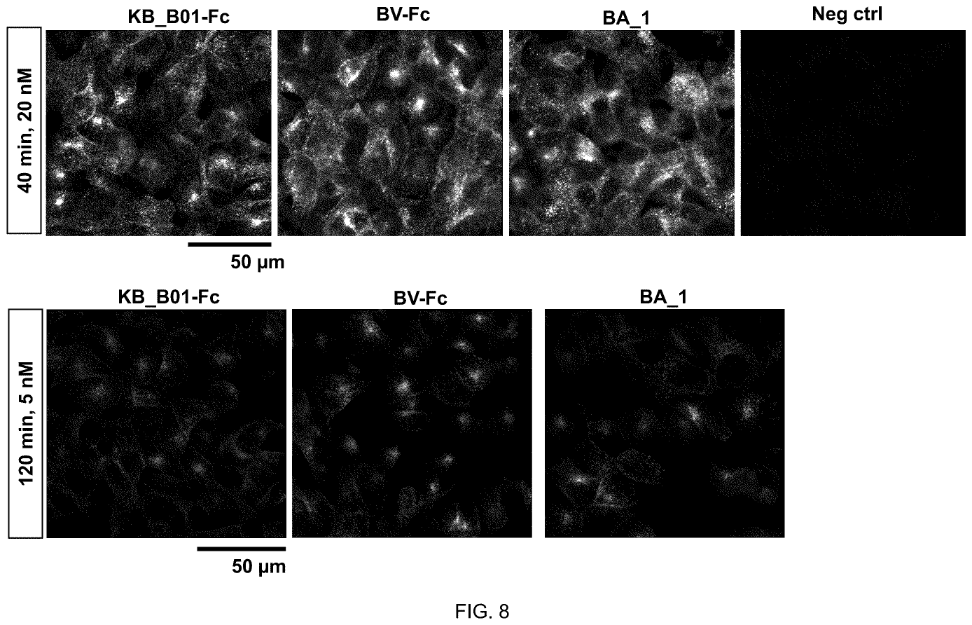

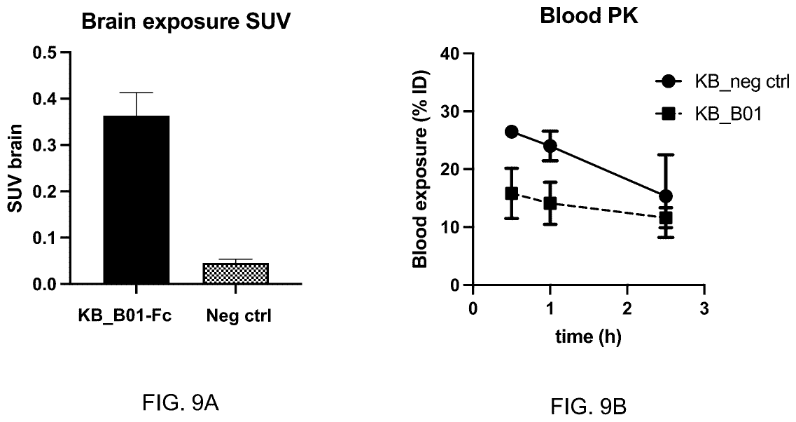

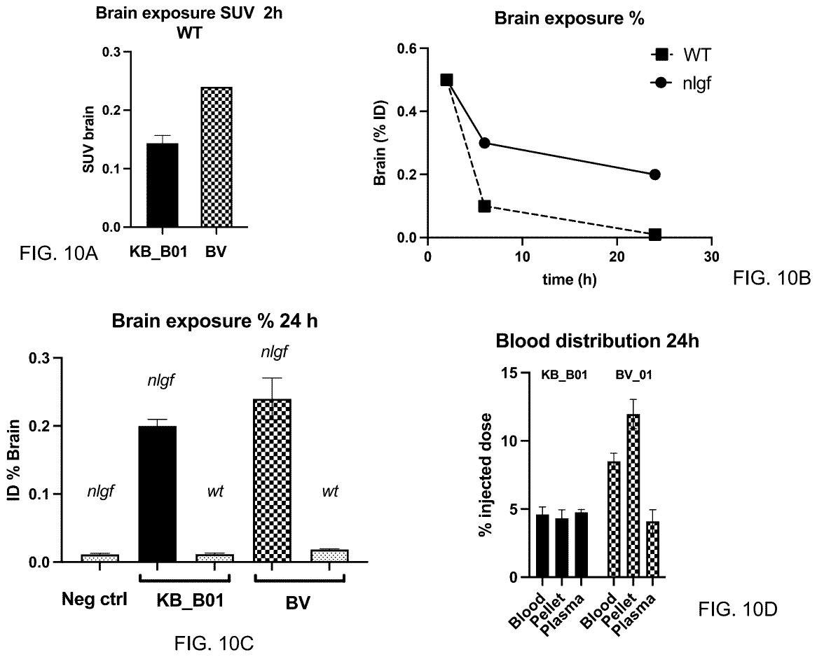

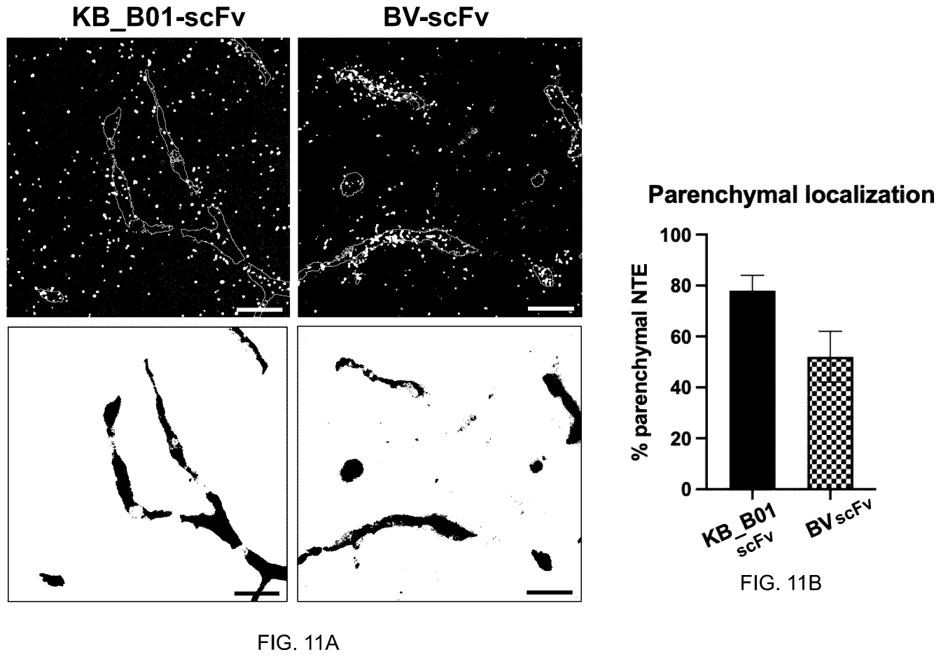

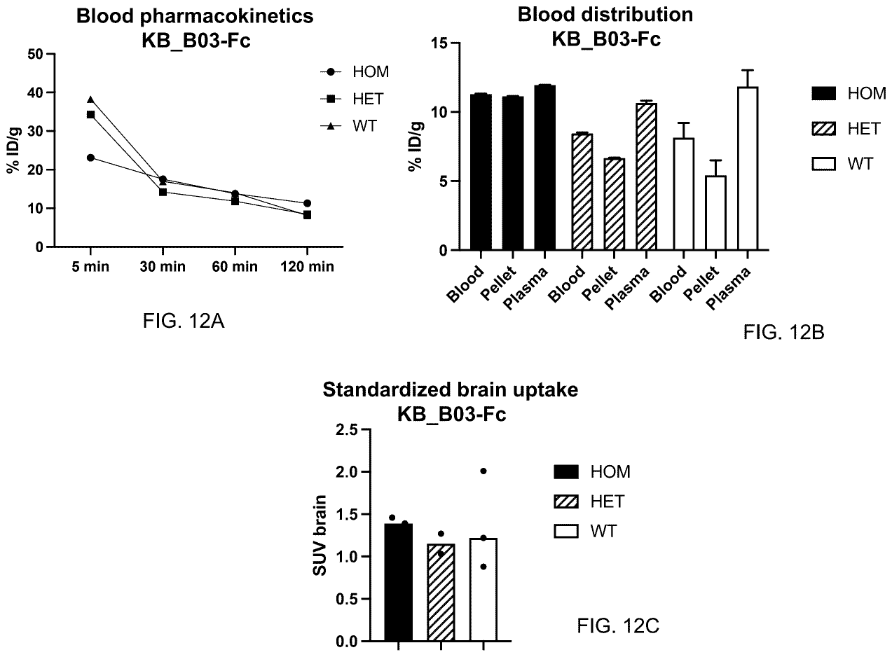

Immunization for antibody generation in llama (Lama glama) animals (N=2) was performed according to the ModiPhage™ method at Modiquest Research BV (the Netherlands). Primary immunization was performed in two llama animals with an adaptive immunization protocol based on the monitoring of specific immune response to the immunogens hTfR1 and mTfR1 (produced as described in Example I). Immunization was made with a dose of 500 μg protein, intramuscular (i.m.) as shown in FIG. 1A on Day 1 (hTfR1), Day 21 (mTfR1) and Day 42 (hTfR1). On Day 54, blood withdrawal was performed to study immune response with ELISA. Serum samples were serially diluted in 3-fold steps (from 1:100 to 1:72900) and screening towards plate-immobilized hTfR1, mTfR1 and control protein bovine serum albumin (BSA), all at 1 μg/ml. Specific binding of anti-llama IgG1, IgG2/IgG3 antibodies were detected with goat anti-rabbit-horse radish peroxidase (HRP) and goat anti-mouse-HRP respectively, detected with 3,3′,5,5′-tetramethylbenzidine (TMB) substrate at absorbance 480 nm in a spectrophotometric plate reader. Two additional doses with 500 μg hTfR1 were delivered on Day 86 and Day 107. On Day 117, blood was drawn and ELISA was performed as described above. One animal was selected for final harvest, also received an additional boost dose with a 1:1 mixture of hTfR1 and mTfR1 (250 μg+250 μg) on Day 120. Four days later (Day 124) peripheral blood was withdrawn for PBL isolation by density gradient centrifugation using Ficoll-Paque™ PLUS ˜1.5×10⁹. RNA extraction was made from ˜0.7×10⁹ PBLs using TRIzol (Thermo Fischer Scientific) Reverse transcription to cDNA was performed by oligo (dT)-primed reverse transcription of 400 μg freshly isolated RNA.

All subsequent steps to construct the phage display library of VHHs was done as described in Pardon et al., A general protocol for the generation of Nanobodies for structural biology. Nat Protoc 9:674-693 (2014), see FIG. 1B. In more detail, cDNA from above was used as a template for a PCR reaction to amplify the IgG2/3 repertoire. A first PCR reaction was performed to amplify all antibodies (IgG1 and IgG2/3) followed by a second nested PCR reaction to amplify and isolate the VHH repertoire, including overhang sites for subcloning into the phagemid pADL10b using primers CALL001 (SEQ ID NO: 52) and CALL002 (SEQ ID NO: 53). An amount of 3 μg of cDNA template was used with the High Fidelity® kit from ThermoFischer Scientific: 10 μl 5× High Fidelity buffer, 1 μl dNTPs (dinucleotide triphosphates; 10 mM of each, 0.2 mM final concentration), 25 pmol per primer (0.5 μM final primer concentration), 0.5 μl Phusion® polymerase (1 U/reaction), ddH₂O (double-distilled water) to 50 μl, 1-2 μl template with cycling program: 98° C. 5 min, 98° C. 30 s (30×), 72° C. 30 s (30×), 72° C. 10 min, 4° C. storage. A total of 36 wells were pooled and then purified using Genejet enzymatic cleanup (Thermo Fischer, cat #K0832) with PCR dimer removal. The mixture was purified using four columns and eluted in 4×20 μl elution buffer. The eluate was loaded on a 1% agarose gel with SYBRSafe DNA GelStain (SigmaAldrich) safe for gel extraction and the extracted product was used as template in a subsequent PCR reaction. Next, nested PCR reactions were used for amplification including pADL10 cloning sites using the following oligonucleotides VHH-R-Sfil (SEQ ID NO: 54) and VHH-F-Sfil (SEQ ID NO: 55). Two PCR mixtures were prepared with 0.5 UM per primer (VHH-F-Sfil and VHH-R-Sfil) and 0.1 UM per primer, respectively and 1 μl template Thermocycling was done as follows: 98° C. 5 min, 98° C. 30 s (20×), 72° C. 1 min (20×), 72° C. min, 4° C. storage. About 50 ng of amplified and gel extracted heavy chain antibody was used as template in each well. All wells were pooled after amplification and purified using Genejet PCR cleanup according to the manufacturers protocol and eluted in elution buffer (10 mM Tris-HCl pH 8.5). PCR product concentration was measured with Nanodrop (result: 160 ng/μl). The products were digested with Sfil restriction enzyme (Thermo Fischer Scientific). DNA fragments at 400 bp were extracted on a 2% agarose gel using Genejet gel extraction.

The pADL10b vector was digested according to manufacturer's instructions using Sfil restriction enzyme (Thermo Fischer Scientific) over-night at 37° C. Digested vectors were purified on a 1% agarose gel and visualized using Sybr Safe (Thermo Fischer Scientific). The procedure was repeated and to a final concentration of 154.6 ng/μl.

After PCR amplification, the library was digested (using Sfil) for 2 h at 50° C. Enzymes were removed using a Genejet PCR cleanup column as above. The resulting material was eluted in 20 μl elution buffer (10 mM Tris-HCl pH 8.5). DNA concentration was measured to 73 ng/μl. The mixture was ligated again. First, the material was heated at 50° C. and slowly cooled back to room temperature (RT, 20-25° C.) followed by incubation over night at 16° C. with shaking. Cleanup was performed as above and eluted in 10 mM Tris-HCl pH 8.5. A total of 1600 ng DNA was used. 1 or 2 μl of ligation mixture was used to electroporate 10 μl TG1 Escherichia coli in 25 μl total reaction volumes (5.48 ms 1.92 kV and 5.48 ms 1.92 kV, respectively). Cells were used immediately after retrieval from the −70° C. freezer and thawing on ice. Transformants were distributed on large agar plates (20 cm) and allowed to grow under standard conditions until colonies present and ready to harvest.

Cells were harvested by adding Luria Broth (LB) medium. Colonies were counted, the library size estimated to 3.2×10⁸. Cultures were prepared of the final library pool after over-night growth on agar plates. The cultures were shaken at 200 rotations per minutes (rpm) at 37° C. for 2 h. Hyperphage was added with a multiplicity of infection (MOI) of 2, for 30 min at 37° C. without shaking. The media was removed by centrifugation at 4500 g for 5 min in Falcon tubes. The cells were then re-suspended in an equal volume of Terrific Broth (TB) medium supplemented with 100 μg/ml ampicillin, 50 g/ml kanamycin and 1 mM isopropyl β-d-1-tiogalaktopyranosid (IPTG). Cultures were shaken over-night at 30° C. at 200 rpm.

5 mL cultures were prepared in LB from over-night cultures of selected clones in 15 mL falcon tubes. supplemented with 100 μg/ml ampicillin, glucose was omitted. Over-night cultures were diluted 1:100 in 5 ml LB and grown for 2 h at 150 rpm until the cultures were turbid. 1 μl hyperphage helper phage (Hyperphage, titer: 1.4×10¹² cfu/ml, Progen) was added to each culture. The helper phage was allowed to infect for 30 min at 37° C. Phage-containing LB was discarded by centrifugation at 4500 g. Bacterial pellets were re-suspended in 5 ml TB medium supplemented with 100 μg/ml ampicillin, 50 μg/ml kanamycin and 1 mM IPTG. The cultures were shaken at 200 rpm and 30° C. for over-night. Cultures were centrifuged at 4500 g for 4 min to remove the bacteria, adding 0.25×V of 20% PEG8000/2.5 M NaCl was added and incubated on ice for 2 h in centrifuge tubes. The phage/bacteria were pelleted at 15000 g for 10 min and re-suspended in 0.5 ml PBS. The phage-containing supernatants were transferred to separate microcentrifuge tubes pre-blocked with PBS/casein.

Panning with Dynabeads® with Biotinylated TfR1

Biotinylation of hTfR1, cTfR1 and mTfR1 was performed according to standard procedures with a biotinylation kit EZ-link Sulfo NHS PEG4 (Thermo Fischer Scientific) using 1 mg target protein with a mixture of 2 mM biotin at a 1:1 molar ratio at pH 8. Free biotin was removed by dialysis in a Pierce 3 mL 10 kDa MWCO (molecular weight cut-off) dialysis unit, at 4° C. with 3×1 h with 1 L PBS buffer at pH 8. The protein was concentrated to 850 μl with Amicon spin concentration 10 kDa MWCO. The phage supernatants were pre-blocked with PBS/casein and mixed with 100 nM of biotinylated hTfR1, cTfR1 or mTfR1, and incubated for 1 h. Bead blocking was done using 50 μl Streptavidin Dynabeads® (M280, Invitrogen, Thermo Fischer Scientific) with hTfR1 1.1 mg/ml and mTfR1 (1 mg/mL) and washed with PBS with 0.15% Tween® 20 (PBST). The beads were re-suspended in 1 ml ELISA blocker (PBS/casein ELISA blocker (Thermo Scientific) and incubated for 30 min in a rotamixer at RT. Blocker was replaced by the phage+target mixture, incubated for 30 min at RT with gentle rotation to capture the biotinylated TfR1 on the beads. Beads were washed 5 times with PBS. Both mTfR1 and hTfR1 captures were eluted in 400 μl PBS with 0.25 mg/ml trypsin.

Phages were trypsinated for 30 min at RT before titration and rescue. A TG1 Eschericia coli culture was started by inoculating a 10 ml culture in 10 ml of LB medium with a single colony of TG1 grown on a M9 minimal agar plate. The culture was incubated at 37° C. at 200 rpm until the OD₆₀₀ had reached log phase (around 4 h, OD₆₀₀ at around 0.6). The culture was kept on ice, dilutions (1-10 times) prepared and mixed with 100 μl TG1 and incubated for 5 min before plating the mixtures on LB Lennox agar plates (Merck-Sigma Aldrich) supplemented with 2% glucose and 100 μg/ml ampicilin. The input was first trypsinated (1 mg/ml trypsin) by mixing 75 μl of the phage stock with 25 μl and incubated at RT for 30 min before dilution and titration. The entire remaining phage outputs were mixed with 2 ml TG1 E. coli each and incubated at RT without shaking for 5 min. The mixtures were plated on two large LB Lennox agar plates each and allowed to dry completely before incubating at 37° C. in plastic bags.

The procedure for selection with capture Dynabeads (and subsequent rescue) was repeated in three subsequent rounds with either hTfR1, cTfR1 or mTfR1 at 100 nM, 25 nM, 10 nM and 5 nM (the last two in the presence of 1 μM human transferrin (recombinant made in rice, Sigma Aldrich, human Transferrin), according to Table 1 below. After the 4^{th} round of selection, 25 colonies were randomly picked from agar plates representing each track and elution method (trypsin or citrate/phosphate buffer of pH of 5.5 with 0.15% Tween® 20), sent for sequencing with primer pADL10fwd at Karolinska Institute (Sweden) genetic analysis core facility.

TABLE 1
Biopanning scheme for selection

| Panning | | | | | | | | | Concentration | Transferrin |
|---|---|---|---|---|---|---|---|---|---|---|
| round | 1 | | 2 | | 3 | | 4 | | TfR1 | (Tf) |
| Selection 1 | hTfR1 | | hTfR1 | | mTfR1 | | mTfR1 | | 100 nM | No |
| Selection 2 | hTfR1 | | mTfR1 | | mTfR1 | | hTfR1 | | 25 nM | No |
| Selection 3 | cTfR1 | | cTfR1 | | mTfR1 | | mTfR1 | | 10 nM | 1 μM |
| Selection 4 | hTfR1 | | mTfR1 | | mTfR1 | | hTfR1 | | 5 nM | 1 μM |
| Elution | pH | tryp | pH | tryp | pH | tryp | pH | tryp | — | — |
| method | | | | | | | | | | |

| Clones | 25 | 25 | 25 | 25 | 25 | 25 | 25 | 25 | Total picked clones N = 200 |
|---|---|---|---|---|---|---|---|---|---|
| (n=) | | | | | | | | | |

### Example III—Binding with ELISA to Select VHHs Binding the hTfR1

Selected phage samples were diluted 1:10 in block buffer casein ELISA blocker (ThermoFischer Scientific), 20% v/v PEG8000 in 2.5 M NaCl before the ELISA. The targets (hTfR1, cTfR1, mTfR1) were coated at 1 μg/ml in PBS pH 7.4 overnight at 4° C. in 96-well Greiner plates (non-treated), 100 μl. The coating buffer was discarded and replaced by 100 μl block solution. The plate was covered and incubated at RT for 2 h, 900 rpm. Anti-human TfR1 antibody BA3 (MEM189, Sigma, 1 mg/ml) and anti-mTfR1 (8D3, Novus biologicals, 1 mg/ml) were diluted in block buffer to 100 nM starting concentration and diluted 1:2 in series by transferring 100 μl to 100 μl block buffer. Plates were incubated for 1 h at RT, 900 rpm, Wells were washed 3×300 μl in PBST (PBS with 0.1% Tween® 20) using the 50TS microplate washer (Biotek). Anti-M13 antibody (Anti-PVIII GE Healthcare) diluted 1:4000 in block buffer, 100 μl was added to target-coated wells, followed by incubation for 1 h at RT, 900 rpm. Washing with PBST was performed. The secondary anti-mouse antibody (Sigma) 1:4000 in block buffer, incubated 1 h at RT, 900 rpm. Washes in PBST was followed by development with TMB substrate. The reaction was stopped with addition of 2 M H₂SO₄ to each well, readings for absorbance at 450 nm was measured using a SpectraMax 3000 (Molecular Devices). One clone (KB_B01) used in the following examples was selected based on cross-reactive binding in the ELISA to both cTfR1 and hTfR1. The criteria set for selection was reactivity to both human and cynomolgus receptor >10 times above blanks (negative controls). The selection criteria in this workflow was also based on non-competition with Tf for the Tf binding site (see Example II), which is not a preferred binding site for brain transport of therapeutics.

### Example IV—Production of Free VHHs or Fusion Variants

Selected VHH clone (KB_B01) was produced as single VHH units (12-14 kDa), see FIG. 2, with a C-terminal 6×His tag (SEQ ID NO: 56) followed by a “C-tag” (C-terminal amino acids EPEA, SEQ ID NO: 57) ordered and codon-optimized from Genscript Inc. using expression Vector pEt-22b (+). Protein was obtained from the periplasmic space in Escherichia coli using standard methods and purified using one-step purification on a Ni-column.

Human Fc-region was genetically fused to VHHs with a GG spacer creating a bivalent TfR1-binding functional entity, see FIG. 2.

In more detail, a VHH Fc fusion protein (SEQ ID NO: 59) between human IgG1 Fc (SEQ ID NO: 58) and KB_B01 (SEQ ID NO: 30) with signal peptide from murine IgG kappa light chain (SEQ ID NO: 81) was cloned into a vector pcDNA3.4 backbone with codon-optimized sequences (performed at GeneArt, Thermo Fischer Scientific) and transiently expressed in Expi293™ cells (Thermo Fischer Scientific), 200 mL cultures grown in standard conditions and harvested 6 days after transfection. Purification of culture medium was done on a MabSelect Sure™, polished in a HiLoad Superdex 200 and yielded >99% pure fractions of dimers VHH-Fc fusions, as verified by analytical size-exclusion chromatography (SEC). The same process was done to produce a fusion protein for a benchmark VHH (SEQ ID NO: 80) as disclosed in WO 2020/144233 and human IgG1 Fc. This benchmark VHH-containing fusion protein is referred to as BV herein.

Further, KB_B02 (SEQ ID NO: 31), KB_B03 (SEQ ID NO: 32), KB_B04 (SEQ ID NO: 33), KB_B05 (SEQ ID NO: 34), KB_B06 (SEQ ID NO: 35), KB_B07 (SEQ ID NO: 36) KB_B08 (SEQ ID NO: 37), KB_B09 (SEQ ID NO: 46) and KB_B10 (SEQ ID NO: 47) were produced in Turbo-CHO™ High Performance platform systems following sequence optimization and cloning into proprietary plasmids at GenScript Biotech Corporation in a volume of 4 mL, based on the same human IgG1 Fc and GG linker sequences as above and a signal peptide (SEQ ID NO: 79), yielding the following Fc-fusions KB_B02-Fc (SEQ ID NO: 60), KB_B03-Fc (SEQ ID NO: 61), KB_B04-Fc (SEQ ID NO: 62), KB_B05-Fc (SEQ ID NO: 63), KB_B06-Fc (SEQ ID NO: 64), KB_B07-Fc (SEQ ID NO: 65), KB_B08-Fc (SEQ ID NO: 66), KB_B09-Fc (SEQ ID NO: 67) and KB_B10-Fc (SEQ ID NO: 82). Final samples were harvested on Day 6 after transfection, purified on protein A-based systems at Genscript yielding 95-99% purity as verified by either SDS-PAGE or analytical size-exclusion chromatography (SEC).

Functional fusion proteins were produced in the form of fusions with VHH and a single-chain variable domain (scFv). Such a fusion protein has monomeric TfR1 binding but potential for bispecific binding, due to the presence of the scFv domain, which is able to bind a different target than TfR1 or a different epitope on the same target. As a functional and representative scFv unit was chosen the amyloid beta-binding murine antibody 3D6. The scFv of the 3D6 antibody was made up by VH (SEQ ID NO: 68) and VL (SEQ ID NO: 69) was fused to either the N-terminus or the C-terminus of KB_B01 and BV joined by a 3×(G₄S) linker (SEQ ID NO: 70) resulting in KB_B01-scFv (SEQ ID NO: 71) or scFv-BV (SEQ ID NO: 72), BV-scFv (SEQ ID NO: 73) or scFv-KB_B01 (SEQ ID NO: 74). Genetic constructs were sequence optimized and synthesized and cloned into custom plasmids by GenScript Biotech Corporation and expressed in transiently transfected TurboCHO™ High Performance platform cells. Purification was performed with HiTrap™ FF Crude (Cytiva) followed by HiLoad™ 26/600 Superdex (Cytiva) according to standard procedures.

Benchmark antibody 128.1 (BA1) was expressed as a full length human IgG1 (SEQ ID NO: 75) in transiently transfected FreeStyle™-293 using vector pcDNA3.4 and harvested from medium, purified in one-step purification on Protein A resin. Benchmark antibody JCR-IgG (BA2) (SEQ ID NO: 76) was designed based on information in U.S. Pat. No. 9,994,641 as an IgG1 antibody reactive to hTfR1 through its Fab′2 (SEQ ID NO: 51). Heavy chain (HC) and Light chain (LC) sequences optimized and cloned into separate plasmids and transfected at expressed in transiently transfected ExpiCHO™ cells with standard reagents and harvested on Day 8. The protein was purified with MabSelect Sure™ (Cytiva) followed by Superdex 16/60 (Cytiva) and analyzed according to standard procedures known to one skilled in the art.

### Example V—Binding Characteristics of KB_B01

Binding of the llama VHH KB_B01 to the hTfR1 and cTfR1 was identified during phage display panning towards hTfR1, and cTfR1 by ELISA. Binding kinetics was analyzed further by surface plasmon resonance (SPR) and biolayer interferometry (BLI). VHH KB_B01 was tested for binding affinity to the target human protein and for lack of interference in affinity by transferrin (Tf). SPR analyses were performed with on a Biacore T200 or Biacore 8K instrument (Cytiva, Sweden). SPR Series S Chip SA and Series S CM5 chips or Series S protein A chips (Cytiva, Uppsala Sweden) were used. Biotinylated transferrin was coupled to SA chip.

Human TfR1, mTfR1, cTfR1 were all biotinylated with biotinylation kit: EZ-Link NHS-PEG4-Biotin, No-Weigh format (Thermo Fisher Scientific) according to manufacturer's instructions. Stock biotin solution vials were dissolved and stored at −70° C. in DMSO to a concentration of 20 nM. Such tube was thawed and freshly diluted to 2 mM in ddH₂O and mixed with TfR1 protein in a 2× molar excess of biotin in PBS in total volume of 200 μL. The mixture was incubated at 25° C. for 45 minutes with shaking and then at 4° C. overnight. Buffer exchange was performed using a Nap5 column (Cytiva) according to manufacturer's instructions. Final elution volume was 750 μL. A PBS buffer at pH 8 was used for buffer exchange. Protein concentration was determined using absorbance 280, resulting in >75% yield.

Binding characteristics of monomeric VHH KB_B01 along with the benchmark full length antibody BA1 were evaluated by SPR with a Biacore T200 instrument using single-cycle kinetics. Human Tf was amine-coupled on a CM5 chip (to about 3000 RUs) and subsequently hTfR1 was captured on immobilized human transferrin (hTf). Human TfR1 was loaded on hTf by injection at 100 nM followed by injection of increasing concentrations of analyte (VHH KB_B01 or BA1) ranging from 6.25 to 100 nM in 2-fold increments. The contact time was 150 s for each concentration at 30 μL/s with a final dissociation step of 500 s. At the end of each single cycle run, the hTf surface was fully regenerated by an injection of 10 mM glycine pH 2 for 30 s. The data with respect to the association rate constant (ka), the dissociation rate constant (kd) and the equilibrium dissociation constant (K_{D}) was evaluated using Biacore T200 software evaluation tool applying a Langmuir 1:1 model.

Monomeric VHH KB_B01 and benchmark antibody BA1 bound to hTfR1 (Table 2 and FIGS. 3A, 3B), confirming human non-Tf interfering binding. Since the hTfR1 was immobilized through loading on hTf, the binding of the analytes implies that neither KB_B01 nor BA1 competes with hTf for binding to hTfR1. Furthermore, the results from comparing KB_B01 to BA1 regarding affinity and binding kinetics showed that monomeric KB_B01 bound to pre-complexed hTfR1-Tf with a 166-fold lower affinity (K_{D}) of 3.31 nM compared to the bivalent BA1 (K_{D}=0.0199 nM) owing to a dramatic difference in dissociation rate (kd). It has been shown that too high affinity may cause the TfR1-binding antibodies and any protein fused to it to be retained in the vasculature and/or cause lysosomal degradation of TfR1-anti-TfR1 complexes (Bien-Ly N, et al., Transferrin receptor (TfR) trafficking determines brain uptake of TfR antibody affinity variants. J Exp Med. 211 (2): 233-244 (2014). Hence, VHH KB_B01 has binding characteristics that make it more suitable as brain drug transporter due to its lower affinity to hTfR1 as compared to BA1.

TABLE 2
Affinity of monomeric VHH and full-length benchmark
antibody BA1 for hTfR1 measured by SPR

| Clone | TfR1 spec | ka (M⁻¹s⁻¹) | kd (s⁻¹) | K_{D} (M) |
|---|---|---|---|---|
| KB_B01 | Hum | 5.97 × 10⁵ | 1.98 × 10⁻² | 3.31 × 10⁻⁹ |
| BA1 | Hum | 2.82 × 10⁶ | 5.63 × 10⁻⁵ | 1.99 × 10⁻¹¹ |

### Example VI—Characterization of Fc-Fused VHH KB_B01

Affinity measurements were carried out by SPR on a Biacore T200 (Cytiva), by loading KB_B01-Fc, BV-Fc and BA2 (Example IV) on protein A chip (Cytiva) at a high degree (100 nM for 60 s). Subsequently these were exposed to increasing concentrations of hTfR1 ranging from 0.16 to 100 nM in 5-fold increments. The data is summarized in Table 3 and sensorgrams are presented in FIGS. 4A-41. The dimeric KB_B01-Fc bound to hTfR1 with a K_{D} value of 0.176 nM (Table 3, FIG. 4A), i.e., 19-fold higher affinity compared to the free monomeric KB_B01 binding to immobilized hTfR1 (K_{D}=3.31 nM, see Table 2). As controls, benchmark BV-Fc and BA2 were run under the same conditions and displayed affinities (K_{D} values) of 0.00228 nM (FIG. 4D) and 0.0249 nM (FIG. 4G), respectively. Cynomolgus and mouse TfR1 were also run under the same conditions and KB_B01-Fc bound mTfR1 with an affinity of 2.82 nM (FIG. 4C). The benchmark BV-Fc bound mTfR1 with high affinity (K_{D}) of 0.444 nM (FIG. 4F) meaning that KB-B01 bound mTfR1 with 6.4-fold lower affinity than the benchmark BV when both were in the form of Fc-fusions. In contrast, the benchmark antibody BA2 did not bind mTfR1 with detectable signal (FIG. 41). This experiment shows that KB_B01 has the desirable attributes of low nM affinity range and also an interesting profile of K_{D} in terms of release (OFF) to the receptor.

Furthermore, KB_B01 exhibited a broad species cross-reactivity, a feature highly desirable from a translational point of view when predicting both pharmacokinetics and pharmacodynamics from mouse to man.

TABLE 3
Binding kinetics of KB_B01 and BV as dimeric Fc
fusion to human, cynomolgus and mouse TfR1 using SPR

| Clone | TfR1 species | ka (M⁻¹s⁻¹) | kd (s⁻¹) | K_{D} (M) |
|---|---|---|---|---|
| KB_B01-Fc | Human | 5.50 × 10⁵ | 9.67 × 10⁻⁵ | 1.76 × 10⁻¹⁰ |
| | Cynomolgus | 7.65 × 10⁵ | 8.18 × 10⁻⁵ | 1.07 × 10⁻⁹ |
| | Mouse | 8.35 × 10⁶ | 2.35 × 10⁻² | 2.82 × 10⁻⁹ |
| BV-Fc | Human | 5.51 × 10⁵ | 1.26 × 10⁻⁶ | 2.28 × 10⁻¹² |
| | Cynomolgus | 1.91 × 10⁵ | 2.61 × 10⁻⁷ | 1.36 × 10⁻¹² |
| | Mouse | 3.82 × 10⁵ | 1.70 × 10⁻⁴ | 4.44 × 10⁻¹⁰ |
| Antibody | Human | 1.56 × 10⁶ | 3.89 × 10⁻⁵ | 2.49 × 10⁻¹¹ |
| BA2 | Cynomolgus | 2.45 × 10⁵ | 3.34 × 10⁻⁴ | 1.36 × 10⁻⁹ |

| | |
|---|---|
| Mouse | No apparent binding |

To further investigate the effect of soluble human transferrin on binding to human TfR1, SPR as above was performed in the presence and absence of human Tf. KB_B01-Fc fusion protein, benchmark BV-Fc, BA1 and BA2 were immobilized on protein A sensors (Cytiva) and subsequently exposed to increasing concentrations ranging from 0.156 to 40 nM (4-fold increments) of the receptors in the absence or presence of a saturating concentration of 250 nM hTf. High affinity for the hTfR1 was retained in the presence of 250 nM of hTf suggesting that KB_B01-Fc and the two benchmark antibodies BA1 and BA2 did not interfere with Tf binding to the human TfR1 (Table 4, FIGS. 5A-5F). This experiment shows that KB_B01 has the desirable and unique attributes for using the TfR1 molecule in man to increase delivery of therapeutic or diagnostic proteins to the CNS.

An interesting difference between KB_B01 and the benchmark VHH BV was that the binding of BV-Fc to hTfR1 was more affected by the presence of hTf than the binding of KB_B01-Fc. The affinity to hTfR1 dropped almost 390 times for BV-Fc but merely 10 times for KB_B01-Fc in the presence of 250 nM hTf versus without hTf.

TABLE 4
Effect of the presence of transferrin on binding to human TfR1.

| Clone | hTf | ka (M⁻¹s⁻¹) | kd (s⁻¹) | K_{D} (M) |
|---|---|---|---|---|
| KB_B01-Fc | No | 6.51 × 10⁵ | 4.34 × 10⁻⁵ | 6.67 × 10⁻¹¹ |
| | 250 nM | 1.52 × 10⁵ | 1.01 × 10⁻⁴ | 6.64 × 10⁻¹⁰ |
| BV-Fc | No | 5.38 × 10⁵ | 2.86 × 10⁻⁷ | 5.31 × 10⁻¹³ |
| | 250 nM | 5.49 × 10⁵ | 1.13 × 10⁻⁴ | 2.07 × 10⁻¹⁰ |
| BA1 | No | 8.82 × 10⁵ | 8.87 × 10⁻⁵ | 1.01 × 10⁻¹⁰ |
| | 250 nM | 6.92 × 10⁵ | 5.77 × 10⁻⁵ | 8.34 × 10⁻¹¹ |
| BA2 | No | 3.15 × 10⁶ | 5.65 × 10⁻⁵ | 1.80 × 10⁻¹¹ |
| | 250 nM | 1.07 × 10⁶ | 1.42 × 10⁻⁴ | 1.33 × 10⁻¹⁰ |

### Example VIII—Bioinformatics and Analysis of Additional Clones

KB_B01 was aligned to all the clones sequenced based on_initial screening of clones (panning) using Jalview multiple sequence alignment editor and analysis workbench version 2 (Barton group, University of Dundee) software. Based on sequence identity to KB_B01, and confirmed positive binding in ELISA, nine additional clones: KB_B02 to KB_B10 (see FIG. 6 for alignment) were selected and produced as fusions to human IgG1 Fc (Example IV). One clone, with high known interference on Tf binding, was selected to use as reference KB_BC.

Based on the above, KB_B02 to KB_B10 were produced as fusions to human IgG1 Fc (see Example IV) and tested for affinity for the hTfR1 and mTfR1 using SPR and immobilizing the dimeric Fc-fused VHHs to protein A chips as described above. Briefly, using Biacore 8K and single-cycle kinetics, VHH-Fc constructs were diluted to 5 nM and captured on protein A chip (Cytiva), eight at a time for 120 s at 5 μL/s followed by injection of increasing concentrations of receptors ranging from 0.8 to 500 nM (contact time 120 s, dissociation 600 s) in five-fold increments. After each full single-cycle kinetics cycle, the surfaces were regenerated by 10 mM glycine, pH 1.5 for 30 s at 30 μL/s. Kinetic data for KB_B02 to KB_B10 as Fc fusions are presented in Table 5. KB_B02 to KB_B10 all bound hTfR1 with high affinities defined by K_{D} values ranging from below 0.00309 nM to 0.451 nM (Table 5). Affinities for mTfR1 ranged from below 0.0171 nM to 89.7 nM.

TABLE 5
Binding kinetics of KB_B02 to KB_B10 as dimeric
Fc fusion to human and mouse TfR1 using SPR

| Clone | TfR1 spec | ka (M⁻¹s⁻¹) | kd (s⁻¹) | K_{D} (M) |
|---|---|---|---|---|
| KB_B02 | Human | 1.96 × 10⁵ | 4.03 × 10⁻⁵ | 2.06 × 10⁻¹⁰ |
| | Mouse | 8.09 × 10⁴ | 2.11 × 10⁻⁴ | 2.61 × 10⁻⁹ |
| KB_B03 | Human | 3.03 × 10⁵ | 2.13 × 10⁻⁵ | 7.01 × 10⁻¹¹ |
| | Mouse | 6.75 × 10⁴ | 4.58 × 10⁻⁵ | 6.79 × 10⁻¹⁰ |
| KB_B04 | Human | 2.34 × 10⁵ | 1.16 × 10⁻⁶ | 4.95 × 10⁻¹² |
| | Mouse | 9.28 × 10⁴ | 3.88 × 10⁻⁴ | 4.19 × 10⁻⁹ |
| KB_B05 | Human | 2.47 × 10⁵ | 3.48 × 10⁻⁶ | 1.41 × 10⁻¹¹ |
| | Mouse | 8.35 × 10⁸ | 6.99 × 10¹ | 8.37 × 10⁻⁸ |
| KB_B06 | Human | 2.47 × 10⁵ | 3.79 × 10⁻⁶ | 1.53 × 10⁻¹¹ |
| | Mouse | 6.53 × 10⁸ | 5.85 × 10¹ | 8.97 × 10⁻⁸ |
| KB_B07 | Human | 2.43 × 10⁵ | 4.48 × 10⁻⁶ | 1.84 × 10⁻¹¹ |
| | Mouse | 7.07 × 10⁸ | 4.79 × 10¹ | 6.78 × 10⁻⁸ |
| KB_B08 | Human | 1.57 × 10⁵ | 7.09 × 10⁻⁵ | 4.51 × 10⁻¹⁰ |
| | Mouse | 4.58 × 10⁸ | 5.34 × 10¹ | 1.16 × 10⁻⁷ |
| KB_B09 | Human | 2.17 × 10⁴ | 7.53 × 10⁻⁷ | 3.47 × 10⁻¹¹ |
| | Mouse | 7.98 × 10³ | 1.04 × 10⁻⁴ | 1.30 × 10⁻⁸ |
| KB_B10 | Human | 3.23 × 10⁴ | <1 × 10⁻⁷ | <3.09 × 10⁻¹² |
| | Mouse | 5.83 × 10³ | <1 × 10⁻⁷ | <1.71 × 10⁻¹¹ |

Next, epitope binning was performed by pairwise testing binders for the hTfR1 in a combinatorial manner using SPR on a Biacore 8K. KB_B01, KB_B04, KB_B05, KB_B08, KB_B09 and KB_B10 along with benchmark antibodies BA1 and BA2 were immobilized by amine coupling onto CM5 chips series S (Cytiva). Yet another VHH-Fc known to bind at a distinct epitope KB_BC, was also immobilized. Proteins to be immobilized were diluted to 25 μg/mL in sodium acetate at pH 5.5 and bound proteins to a SPR signal of about 6000 resonance units (RUs). 50 nM of the ectodomain of hTfR1 was used for the first injection (contact time 120 s, 10 μL/s) and 100 nM of each analyte in the second injection; KB_B01 to KB_B10, BA1, BA2, KB_BC, mouse transferrin (mTf) and human transferrin (hTf) over a contact time of 150 s, 10 μL/s. Following each cycle, the surfaces were regenerated by 10 mM glycine at pH 2.1 for 30 s at 30 μL/s. The data were analyzed using Biacore Insight Evaluation package version 4.0.8.20368. Results show that KB_B01 to KB_B10 all compete with one another for binding to hTfR1, thus, forming a distinct epitope (Table 6). Antibody BA1, known to bind to the apical domain of the hTfR1 (Helguera G. et al, An antibody recognizing the apical domain of human transferrin receptor 1 efficiently inhibits the entry of all new world hemorrhagic Fever arenaviruses. J Virol. 86 (7): 4024-4028 (2012)), competed with BA2 but not KB_B01 to KB_B10. The control VHH-Fc molecule KB_BC did neither block any of the other anti-TfR1 molecules nor BA1 and BA2 but instead completely prevented mTf and hTf from binding to hTfR1. The KB_BC control shows that all others do not compete with Tf and, thus, KB_B01 through KB_B10 have desirable properties for brain transport.

In order to prove functionality when fused to a non-Fc protein, KB_B01 and benchmark BV were produced as a fusion to a single-chain variable domain (scFv of the type VH-VL with the heavy chain located in the amino-terminus and the light chain in the carboxy-terminus) located either in the amino-terminus (scFv-KB_B01) or carboxy-terminus (KB_B01-scFv) see Example IV and FIG. 2 for details. In order to verify that these fusion proteins still bound to hTfR1, binding kinetics was studied by SPR using an 8K Biacore, Briefly, the receptor hTfR1 was immobilized to a CM5 chip (Cytiva) by amine-coupling and exposed to increasing concentrations of either scFv-KB_B01 or KB_B01-scFv ranging from 0.25 to 64 nM in 4-fold increments using single-cycle kinetics. Similarly, the benchmark VHH BV was produced as analogous fusion proteins; scFc-BV and BV-scFv, respectively and tested in the same experiment but with a slightly wider concentration range from 0.08 to 50 nM in 5-fold increments. The kinetic binding data and sensorgrams are presented in Table 7 and FIGS. 7A-7D. Thus, it can be concluded that KB_B01 can be coupled to another protein in either the amino terminus (scFv-KB_B01, K_{D} 1.80 nM) or the carboxy terminus (KB_B01-scFv, K_{D} 0.845 nM) without loss affinity for the hTfR1. In fact, the affinities were slightly higher compared to monomeric KB_B01 complexed with hTf (K_{D} 3.31 nM, Table 2) suggesting that KB_B01 can be used as a BBB transport vehicle fused to a therapeutic molecule without loss of function.

TABLE 7
Binding kinetics of KB_B01 and benchmark
VHH BV fusions to a scFv to hTfR1 using SPR

| Construct | ka (M⁻¹s⁻¹) | kd (s⁻¹) | K_{D} (M) | Rmax (RU) |
|---|---|---|---|---|
| scFv-KB_B01 | 1.44 × 10⁵ | 2.59 × 10⁻⁴ | 1.80 × 10⁻⁹ | 16.2 |
| KB_B01-scFv | 5.35 × 10⁵ | 4.50 × 10⁻⁴ | 8.45 × 10⁻¹⁰ | 21.0 |
| scFv-BV | 1.75 × 10⁵ | 1.22 × 10⁻⁴ | 6.98 × 10⁻¹⁰ | 18.5 |
| BV-scFv | 6.17 × 10⁵ | 1.17 × 10⁻⁴ | 1.89 × 10⁻¹⁰ | 24.9 |

### Example VII—TfR1 Mediated Uptake in Human Cells

Next, it was tested whether the KB_B01-Fc-fusion protein could internalize in human TfR1-expressing cells. Adherent HEK293T cells (ATCC), well known to express TfR1, were grown in collagen-coated 96 well plates (ThermoFischer Scientific), seeded at a density of 30.000 cells/well. Cells were grown in DMEM (Dulbecco's modified eagle medium) for 3-5 days, until about 80-90% confluence. KB_B01 as well as the benchmark BV-Fc fusion and known reference binders BA1 and a negative control (an analogous VHH-Fc devoid of binding to hTfR1) were used in this assay.

The cell assay was established based on a titration of test items and a negative control ranging from 0.85 nM to 82.5 nM during May 15, 1930-45 minutes, in 37° C., 5% CO₂. For comparing cellular fate of added proteins after a prolonged exposure here set at 120 minutes, a 30 min incubation was followed by medium replacement by fresh DMEM for 90 minutes (incubated in 37° C., 5% CO₂) followed, after which the medium was replaced a second time. After 120 min, cells were taken to lab bench and immediately washed with PBS (1×) and fixed with 4% paraformaldehyde for 10-15 minutes. After fixation, cells were washed with PBS and permeabilized with 0.1% Triton-X 100™ in PBS and thereafter blocked with 1% bovine serum albumin (BSA) in PBS, followed by addition of solution Alexa Fluor-488-anti human IgG (Fc fragment specific, stock 0.75 mg/ml) (Jackson Immunoresearch). Texas Red-phalloidin 300 U (1:200 dilution of a 10 μg/mL stock) was added to the wells during the last 20 minutes of incubation of secondary antibody. After this, cells were washed at least 3 times with PBS and stored at 4° C. until imaging. Cells were imaged in an inverted Zeiss 710 laser scanning confocal microscope using lasers 488, 555 and 647 and a 20× air objective.

Results from these experiments showed that KB_B01 as well as BV and BA1 were readily taken up into HEK293T cells at various concentrations tested (0.7-85 nM) and at incubation times 5 to 240 minutes. Representative of this, FIG. 8, KB_B01-Fc was taken up efficiently into cells similarly to BV-Fc at 40 minutes, 20 nM concentration. At a longer timepoint, 120 minutes incubation, with a rinse after 30 minutes to remove excess test proteins in the medium, equivalent amounts of KB_B01-Fc, BV-Fc and BA1 were found. A negative control (VHH-Fc, devoid of binding to hTfR1) did not accumulate in cells (FIG. 8) or show any presence at any timepoint or concentration tested, showing that the assay and uptake was TfR1-dependent.

### Example VIII—In Vivo and Ex Vivo Evaluation of VHH-Fc and scFv Fusions

To evaluate the properties of KB_B01 for brain targeting in vivo, KB_B01-Fc was compared to one equivalent VHH-Fc devoid of mTfR1 binding (KB-neg ctrl) in wild-type (WT) mice. Then scFv-VHH fusions (described in Example IV, VI and Table 7) were compared based on N-versus C-terminal orientation of fusion partners and to the negative control. All fusion proteins were radiolabeled with iodine-125 (¹²⁵I) using the chloramine T method as described in Greenwood et al., The preparation of ¹³¹I-labelled human growth hormone of high specific radioactivity. Biochem J 89:114-123 (1963). An amount of 15-80 μg of protein was mixed with 260±26 MBq/μg or 61±2.4 kBq/μg of stock ¹²⁵I (PerkinElmer Inc, Waltham, MA, USA) and 5 μg of Chloramine T (Sigma Aldrich) in PBS. The reaction mixture (110 μL) was allowed to incubate for 90 s before it was quenched with 10 μg of Na-metabisulfite (Sigma-Aldrich). The radiolabeled sample was immediately purified in a Zeba-column (ThermoFischer) of 7 kDA cutoff.

Retained binding to mTfR1 and hTfR1 were validated with ELISA. Briefly, high-binding half-area 96-well plates (Costar 3690, Merck) were coated with ectodomains of hTfR1 and mTfR1 at 1-4 g/ml (in PBS pH 7.4). and left over night at 4° C. Plates were blocked for 1 h using 1% BSA 0.05% Tween®20 (Merck) in PBS. After washing (PBS wash buffer), radiolabeled samples (or original samples) were incubated in diluent buffer (0.1% BSA in PBS-Tween® 20 0.05%) at serial dilutions 1:5, 2 hours or overnight (at) 4° followed by 4 repeated washes and incubation with secondary antibody detecting VHH (anti-VHH secondary antibody (A01861, Genscript Biotech Corporation) at dilution 1:6000. Binding to target (mTfR1, hTfR1) was confirmed in all reported experiments.

Experiments in vivo were performed in C57BL/6 mice aged 12 weeks. Also, to investigate retention to amyloid beta with the bispecific VHH-scFv fusions with the amyloid beta binding 3D6 scFv, the APP^{NL-G-F} model of AD (Nilsson P et al., ACS Chem. Neurosci. 2014, 5, 7, 499-502) with mice aged 13-15 months, was used. Immediately following radiolabeling, mice were administered either VHH-Fc (5 nmol/kg; 3.4 MBq/nmol) or VHH-scFv (5 nmol/kg; 6.8±0.5 MBq/nmol) fusion proteins i.v. via the tail vein. Blood samples (8 μL) were obtained from the tail vein at 5 min, 0.5, 1, 2, 4 and 9 h post injection. Mice were euthanized by sampling blood from the heart prior to transcardial perfusion with 40 mL NaCl during 2.5 min. Timepoints for euthanization post-injection were: 2.5 hours for the VHH-Fc constructs in WT mice, and 2, 6 or 24 hours after injection for VHH-scFv fusions in WT and APP^{NL-G-F} mice to investigate brain retention. Only KB_B01-scFv was used for the 6 hours timepoint. Brain and major organs were dissected. Blood was separated into plasma and blood cell pellet by centrifugation at 10 000×g, 5 min. The brain was separated into the left and right hemispheres. The cerebellum was removed from the left hemisphere, and the remaining tissue of the left hemisphere is hereafter referred to as “brain”. Radioactivity was then measured in brain, blood fractions and major organs using a γ-counter (2480 Wizard™, Wallac Oy PerkinElmer, Turku, Finland). As shown in FIG. 9A, at 2.5 h post-injection, distribution to brain in WT mice had a clearly enhanced SUV of the dimeric VHH-Fc-fusion KB_B01 compared to its negative control VHH-Fc fusion, showing a functional brain targeting mechanism in vivo. Levels in blood of non-mTfR1 binding negative control was higher than KB_B01 as VHH-Fc fusion (FIG. 9B). These experiments show that KB_B01 as Fc-fusion is targeting the brain compartment dependent on its mTfR1-binding properties.

Next, the fate of scFv-VHH fusions was explored at 2 h and 24 hours after injection using both the N-terminal and C-terminal constructs. For KB_B01, the N-terminal positioning of VHH (KB_B01-scFv) showed a favorable brain concentration at 2 h (Table 8). The benchmark BV showed similar relation between orientation with respect to brain concentration and total brain uptake at 2 hours (Table 8).

TABLE 8
Brain concentrations 2 h after injection of
the VHH-scFv3D6 fusion proteins in WT mice

| Protein | % ID/g_{brain} | Protein | % ID/g_{brain} |
|---|---|---|---|
| [¹²⁵I]scFv-KB_B01 | 0.15 ± 0.01 | [¹²⁵I]KB_B01-scFv | 0.52 ± 0.05 |
| [¹²⁵I]KB_neg ctrl | 0.07 ± 0.01 | n/a | n/a |
| [¹²⁵I]scFv-BV | 0.47 ± 0.04 | [¹²⁵I]scFv-BV | 0.94 ± 0.03 |

Mean ± SD.

Next, brain concentrations were evaluated between WT and APP^{NL-G-F} mice at 2 and 24 hours after injection. Retention of the protein through its binding to amyloid beta is critical both for diagnostic and therapeutic efficacy of an active moiety/conjugate. As shown in FIG. 10A, distribution to the brain at 2 hours after injection was higher in BV-scFv than KB_B01-scFv. At 6 hours, only KB-B01 was investigated and showed a clear brain parenchymal retention in APP^{NL-G-F} mice (FIG. 10B). At 24 hours, both KB_B01 and BV showed lower brain concentration than at 2 h (FIG. 10C). A non-specific retention in brain (not binding to amyloid beta) is advantageous here, which is reflected by low levels in WT brains, particularly pronounced in the KB_B01-scFv. As seen in Table 9, KB_B01-scFv has the lowest detectable levels of injected radiolabeled fusion proteins at 24 hours in WT mouse brains. Also, at 24 hours, the ratio of brain concentration between APP^{NL-G-F} and WT mice were higher in KB_B01-scFv compared to benchmark BV-scFv (17.2 vs 13.4, fold difference WT vs APP^{NL-G-F} mice, see Table 9). Furthermore, looking more in detail on peripheral pharmacokinetics, specific distribution to red blood cells (erythrocytes, i.e., pellet), was higher in at 24 h after injection of BV-scFv than KB_B01-scFv, (FIG. 10D).

TABLE 9
Brain concentrations 24 h after injection
of the VHH-scFv3D6 fusion proteins

| | | | Fold |
|---|---|---|---|
| | WT | APP^{NL-G-F} | difference |
| | (% ID/g | (% ID/g | WT vs |
| Protein | brain) | brain) | APP^{NL-G-F} |
| [¹²⁵I]scFv-KB_B01 | 0.04 ± 0.01 | 0.11 ± 0.02 | 2.7 |
| [¹²⁵I]KB_B01-scFv | 0.012 ± 0.01 | 0.20 ± 0.01 | 17.2 |
| [¹²⁵I]KB_neg ctrl | n/a | 0.01 ± 0.01 | n/a |
| [¹²⁵I]scFv-BV | 0.019 ± 0.01 | 0.20 ± 0.01 | 10.6 |
| [¹²⁵I]BV-scFv | 0.018 ± 0.01 | 0.24 ± 0.04 | 13.4 |

Mean ± SD.

### Example IX—Ex Vivo Characterization Evaluation of VHH-scFv Fusions

To investigate VHH distribution between vasculature and brain parenchyma, sagittal cryosections of 20 μm were fixed for 10 minutes in 4% paraformaldehyde (PFA) washed with PBS and blocked with 5% normal goat serum. The sections were incubated with PBS 0.1% Tween® 20 for 15 minutes before overnight incubation with antibody rat-anti-mouse CD31 (BD, #553370), and rabbit-anti-Aβ42 (Agrisera) at 4° C. The following day, sections were washed with PBS and incubated with antibody goat-anti-rat (Alexa 647; Molecular Probes, Thermo Fischer Scientific) and goat-anti-rabbit (Alexa 488, Molecular Probes, Thermo Fischer Scientific). The sections were stored in PBS until the nuclear track emulsion (NTE) procedure (described below) was performed on the same day.

NTE was carried out according to previous published procedures (Gustavsson et al., SPECT imaging of distribution and retention of a brain-penetrating bispecific amyloid-β antibody in a mouse model of Alzheimer's disease, Transl Neurodegener 9:37 (2020)). In darkroom conditions with a safelight according to the manufacturer's instruction, Ilford K5 emulsion (Ilford Photo, Harman Techologies) was melted in a 40° C. water bath and prepared as a 50% solution with MQ-H₂O. Brain sections were submerged in the emulsion for 10 s, followed by air-drying in RT for 2 hours. The sections were stored at 4° C., in a light-sealed box for 4 weeks. Development was done according to the manufacturer's instruction. After NTE, sections with immunostaining and developed emulsion were visualized and systematic images acquired with a Zeiss Observer Z.1 microscope using ZEN software (Carl Zeiss Microimaging GmbH, Jena, Germany) with either widefield illumination (brightfield for NTE) or fluorescence (for CD31) using filter block for emission wavelengths 647. Images were then superimposed (merged) and NTE puncti were quantified within vessel structures or in the parenchymal space, based on macro-based image analysis. Relative parenchymal vs capillary signal (% Parenchyma) in the NTE images analyzed on n=20 images per animal, from one animal per group with the median number of total brain concentrations (i.e., most representative). The quantification was performed with a standardized macro in Fiji (ImageJ) like previously described (Faresjö et al., Brain pharmacokinetics of two BBB penetrating bispecific antibodies of different size, Fluids and Barriers of the CNS 18:26 (2021).

As is shown in FIG. 11A, distribution of KB_B01-scFv to the brain parenchyma without trapping in the blood vessels at 2 h after injection was superior as compared to the benchmark BV-scFv. FIG. 11A shows a representative vessel structure outlined with a thin white line to demonstrate vascular vs parenchymal localization of NTE puncta (white dots). Quantification revealed a preferential distribution to brain parenchyma for KB_B01-scFv of 78±6% vs 52±10% for BV-scFv (FIG. 11B). This means that each dose of KB_B01-scFv reaches the target sites (epitopes) in the brain in a more efficient manner and is most suitable among the constructs tested for using for diagnostic PET imaging for specific imaging of disease biomarkers in situ. Unspecific retention in blood vessels is not desirable for an imaging agent used for PET, and a better parenchymal targeting is preferential also to therapeutics for reaching its target.

### Example X—Generation of Protein a Binding Antibodies

Llama VHHs do not usually bind the protein A based resin PrismA™ but by mutating amino acids in the framework sequences, PrismA™ binding, and thereby protein A binding, VHHs can be produced. To explore the possibility of enabling a protein A binding of KB_B01 and KB_B03 and fusion proteins containing KB_B01 and KB_B03, these two VHHs were mutated according to consensus protein A binding sequences according to Henry et al., A Rational Engineering Strategy for Designing Protein A-Binding Camelid Single-Domain Antibodies, PLoS One 11 (9): e0163113 (2016). KB_B01 was mutated in position 75 from a glutamic acid to a lysine, position 83 from an aspartic acid to an asparagine and in position 84 from an asparagine to a serine (KB_B01: E75K, D83N, N84S, denoted KB_B11 SEQ ID NO: 83) and KB_B03 was mutated in in position 83 from aspartic acid to an asparagine (KB_B03: D83N, denoted KB_B12 SEQ ID NO: 84) as free VHH without His6-tag. KB_B12 was also produced as a fusion protein to scFv (SEQ ID NO: 85) fused to a His6-tag via a short Gly2 linker (GGHHHHHH, SEQ ID NO: 86) to enable Ni-NTA based purification in addition to the PrismA™ based. A positive control known to bind protein A (SEQ ID NO: 87) was also included.

Proteins were produced in CHO cells by ProteoGenix SAS, France. The cDNAs coding for the VHHs (and fusion to scFv) were chemically synthesized with optimization for expression in CHO cells and subcloned in ProteoGenix's proprietary mammalian cells expression vectors. The vectors were transfected in XtenCHO™ cells by XtenCHO transfection protocol. In a total volume of 3.5 mL culture medium was collected 8 days after transfection and purified using Mabselect PrismA resin (Cytiva). Culture media was clarified by filtration (0.22 μm) and loaded on the pre-equilibrated (PBS pH 7.5) PrismA™ resin. Bound VHH was eluted by pH shift using citric acid according to the resin manufacturer's instructions and the eluate was instantly neutralized by addition of 1 M Tris-HCl PH 9.0. Protein concentration was determined by A280 spectrophotometric measurement. The flow through fractions for the scFv fusion construct was subsequently purified by Ni-NTA based purification using IMAC high binding capacity Nickel resin. Equilibration and binding buffer was PBS pH 7.5 and washes and elution were performed by imidazole shift. Protein concentration was determined by A280 nm spectrophotometric measurement and qualitative and quantitative by SDS-PAGE.

Protein concentrations after PrismA™ elution were generally low about 5, 8 and 7 μg/mL culture for the KB_B11, KB_B12 and the scFv fusion KB_B12-scFv, respectively. Subsequent purification by Ni-NTS IMAC of the scFv fusion protein revealed that no material was present in the flow through fraction proving the functionality of the PrismA™ resin-based purification for this protein construct.

PrismA™ resin was used to purify KB_B011, KB_B12 and scFv fusion to KB_B12. Binding to PrismA™ resin for KB_B011, KB_B12 and scFv fusion to KB_B12 along with a ProA positive control (SEQ ID NO: 90) was also verified by SPR with a precoated chip (PrismA chip, Cytiva). A negative control VHH with non-optimized sequence with respect to ProA was also included. The positive and negative controls were produced in E. coli and contained a C-terminal His-tag (SEQ ID NO: 86). KB_B11, KB_B12 and the positive control bound with signals reaching 1232, 651, and 1193 RU, respectively at the highest concentration (500 nM) and KB_B12-scFv bound with signal reaching 545 RU at the highest concentration tested (125 nM) confirming the functionality of VHH-mediated binding to PrismA™ resin (Table 10). No signal above baseline was recorded for the negative control.

TABLE 10
Binding data verifying functionality of PrismA ™ enabled VHHs

| | Mutations vs. | PrismA ™ |
|---|---|---|
| VHH construct | corresponding WT VHH | binding |
| KB_B11 (KB_B01 PrismA ™) | E75K, D83N, N84S | Yes |
| KB_B12 (KB_B03 PrismA ™) | D83N | Yes |
| KB_B12-scFv | D83N | Yes |

### Example XI—In Vivo and Ex Vivo Evaluation of VHH-Fc of KB_B03 in TfR1 Extracellular Domain Humanized Mice (hECD-TfR1-Mice)

To evaluate the properties of KB_B03 for brain targeting in vivo, the distribution properties of KB_B03 was tested in hECD-TfR1-mice of homozygous, heterozygous and wild type genotype. Homozygous mice only express the partially humanized TfR1, i.e., human-mouse chimeric TfR1. Humanized mice expressing a chimeric receptor comprising the extracellular domain of hTfR1 and the intra- and transmembrane domains of mTfR1 were generated at Taconic Biosciences GmbH, Leverkusen, Germany.

A genetically engineered mouse model was generated by Taconic Biosciences GmbH, Leverkusen, Germany, in which the endogenous murine Tfrc gene was partially humanized so that the engineered mice produced a chimeric TFRC protein containing the human TFRC extracellular domain. The targeting vector was assembled using methods known to one skilled in the art using BAC-derived mouse and human genomic DNA fragments and including standard selection cassettes.

Embryonic stem cells (ES) derived from C57BL/6NTac mice, were grown on a mitotically inactivated feeder cell layer comprised of mouse embryonic fibroblasts in ES cell culture medium containing leukemia inhibitory factor and fetal bovine serum. The cells were electroporated with the linearized DNA targeting vector according to methods known to a person skilled in the art. Homologous recombinant clones were isolated using methods known to one skilled in the art. ES cell colonies (ES clones) with a distinct morphology were isolated and analyzed by Southern blotting and/or PCR. Homologous recombination at 3′ and 5′ sites and single integration were confirmed with PCR and Southern blot using conventional methods, known to the one skilled in the art. Homologous recombinant ES cell clones were expanded and frozen in liquid nitrogen.

Generation of chimeras and generation of heterozygotes: After administration of hormones, superovulated BALB/c females were mated with BALB/c males. Blastocysts were isolated from the uterus at dpc 3.5 and placed in a drop of DMEM with 15% FCS (fetal calf serum) under mineral oil. A microinjection-pipette was used to inject 10-15 targeted ES cells into each blastocyst. After recovery, approximately 8 injected blastocysts were transferred to each uterine horn of pseudopregnant NMRI females. The degree of chimerism was assessed in chimeras (F0) by observing coat color contribution of ES cells to the BALB/c host (black/white). In vitro fertilization was performed using oocytes from superovulated C57BL/6NTac females and thawed sperm from previously cryopreserved spermatozoa derived from male chimeras. Fertilized embryos were then transferred into pseudopregnant Swiss Webster recipient females. Germline transmission was identified first by the presence of offspring with black coats (strain C57BL/6NTac) and the confirmed by genotyping of the black offspring via PCR. F1 mice heterozygous for the modified Tfrc allele were mated to produce subsequently homozygous (HOM), heterozygous (HET) and wild type (WT) genotypes, that were used for the studies described below.

KB_B03-Fc was radiolabeled with iodine-125 (¹²⁵I) using the chloramine T method as described in Example VIII. Experiments in vivo were performed in C57BL/6 mice aged 6-10 weeks. Immediately following radiolabeling, mice were administered KB_B03-Fc (5 nmol/kg; 3.4 MBq/nmol) i.v. via the tail vein. Blood samples (8 μL) were obtained from the tail vein at 5 min, 0.5 h, 1 h and terminally at 2 hours post injection. Mice were euthanized 2 hours post injection by sampling blood from the heart prior to transcardial perfusion with 40 mL NaCl during 2.5 min. Brain and major organs were dissected. Blood was separated into plasma and blood cell pellet by centrifugation at 10 000×g, 5 min. The brain was separated into the left and right hemispheres. The cerebellum was removed from the left hemisphere, and the remaining tissue of the left hemisphere is hereafter referred to as “brain”. Radioactivity was then measured in brain, blood fractions and major organs using a g-counter (2480 Wizard™, Wallac Oy PerkinElmer, Turku, Finland). As shown in FIGS. 12A and 12B blood distribution profile in hECD-TfR1 mice showed a faster drop in blood levels at 5 min to 30 min in HOM mice compared to HET mice and WT mice, while blood levels were similar from 30 min-120 mins. Blood compartment analysis showed slightly higher binding to pellet in HOM mice, due to the slightly higher affinity to the hTfR1 than mTfR1 of KB_B_03 (Example VIII, Table 5). Standardized brain uptake (FIG. 12C) showed high uptake in all genotypes, as expected, due to the cross-specific properties of KB_B03. The results show the brain targeting capacity of KB_B03 in a functional hECD-TfR1 in vivo.

TABLE 11
Sequence information

| | | SEQ |
|---|---|---|
| Denotation | Sequence | ID NO: |
| Consensus | GX₁X₂FX₃X₄X₅Y | |
| 1 CDR1 | | |
| Consensus | FTX₆X₇GX₈T | 1 |
| 1 CDR2 | | |
| Consensus | X₉X₁₀LX₁₁X₁₂ | |
| 1 CDR3 | | |
| Consensus | GTX₂FX₃X₄NY | 2 |
| 2 CDR1 | | |
| Consensus | X₉FLX₁₁X₁₂ | |
| 2 CDR3 | | |
| KB_B03- | GTDFSX₄NY | 3 |
| KB_B07 | | |
| CDR1 | | |
| KB_B05- | FTX₆X₇GST | 4 |
| KB_B07 | | |
| CDR2 | | |
| KB_B05- | HFLGD | 5 |
| KB_B07 | | |
| CDR3 | | |
| KB_B03, | GTDFSLNY | 6 |
| KB_B06- | | |
| KB_B07 | | |
| CDR1 | | |
| KB_B06- | FTAX₇GST | 7 |
| KB_B07 | | |
| CDR2 | | |
| KB_B06 | FTATGST | 8 |
| CDR2 | | |
| KB_B07 | FTASGST | 9 |
| CDR2 | | |
| KB_B04- | GTDFSMNY | 10 |
| KB_B05 | | |
| CDR1 | | |
| KB_B05 | FTSTGST | 11 |
| CDR2 | | |
| Consensus | YFLDX₁₂ | 12 |
| 3 CDR3 | | |
| KB_B01- | GTPFX₃NNY | 13 |
| KB_B02 | | |
| CDR1 | | |
| KB_B01- | FTSX₇GX₈T | 14 |
| KB_B02 | | |
| CDR2 | | |
| KB_B01- | YFLDV | 15 |
| KB_B02 | | |
| CDR3 | | |
| KB_B01 | GTPFSNNY | 16 |
| CDR1 | | |
| KB_B01 | FTSGGST | 17 |
| CDR2 | | |
| KB_B02 | GTPFTNNY | 18 |
| CDR1 | | |
| KB_B02 | FTSAGNT | 19 |
| CDR2 | | |
| KB_B03- | YFLDN | 20 |
| KB_B04 | | |
| CDR3 | | |
| KB_B03 | FTTHGDT | 21 |
| CDR2 | | |
| KB_B04 | FTSSGNT | 22 |
| CDR2 | | |
| KB_B08 | GNRFGIEY | 23 |
| CDR1 | | |
| KB_B08 | FTSAGST | 24 |
| CDR2 | | |
| KB_B08 | HYLGD | 25 |
| CDR3 | | |
| FR1 | QVQLQESGX₁₄GX₁₅VQX₁₆GGSLRLSCX₁₇X₁₈S | 26 |
| consensus | | |
| 1 | | |
| FR2 | X₁₉RWYRQAPGX₂₀QREX₂₁VAG | 27 |
| consensus | | |
| 2 | | |
| FR3 | NYX₂₀DSX₂₁KGRFTIX₂₂RDNAX₂₃X₂₄TVYLQMDX₂₅LX₂₆PEDTAVYX₂₇C | 28 |
| consensus | | |
| FR4 | WGQGTQVTVSS | 29 |
| consensus | | |
| KB_B01 | QVQLQESGGGLVQAGGSLRLSCAASGTPFSNNYMRWYRQAPGKQREWVAGFTSGGS | 30 |
| | TNYADSVKGRFTISRDNAEGTVYLQMDNLKPEDTAVYYCYFLDVWGQGTQVTVSS | |
| KB_B02 | QVQLQESGGGLVQAGGSLRLSCTASGTPFTNNYMRWYRQAPGKQREWVAGFTSAGN | 31 |
| | TNYADSVKGRFTISRDNAEGTVYLQMDNLKPEDTAVYYCYFLDVWGQGTQVTVSS | |
| KB_B03 | QVQLQESGGGLVQAGGSLRLSCAASGTDESLNYMRWYRQAPGKQREWVAGFTTHGD | 32 |
| | TNYADSMKGRFTISRDNAKNTVYLQMDSLKPEDTAVYFCYFLDNWGQGTQVTVSS | |
| KB_B04 | QVQLQESGGGLVQAGGSLRLSCALSGTDESMNYMRWYRQAPGKQREWVAGFTSSGN | 33 |
| | TNYPDSVKGRFTISRDNAKNTVYLQMDSLKPEDTAVYYCYFLDNWGQGTQVTVSS | |
| KB_B05 | QVQLQESGGGLVQAGGSLRLSCVASGTDFSMNYMRWYRQAPGKQREWVAGFTSTGS | 34 |
| | TNYPDSMKGRFTISRDNAKNTVYLQMDSLKPEDTAVYYCHFLGDWGQGTQVTVSS | |
| KB_B06 | QVQLQESGGGLVQTGGSLRLSCVVSGTDESLNYLRWYRQAPGNQREWVAGFTATGS | 35 |
| | TNYPDSMKGRFTIGRDNAKNTVYLQMDSLKPEDTAVYYCHFLGDWGQGTQVTVSS | |
| KB_B07 | QVQLQESGGGLVQTGGSLRLSCVVSGTDESLNYLRWYRQAPGKQREWVAGFTASGS | 36 |
| | TNYPDSMKGRFTIGRDNAKNTVYLQMDSLTPEDTAVYYCHFLGDWGQGTQVTVSS | |
| KB_B08 | QVQLQESGRGVVQAGGSLRLSCAASGNRFGIEYMRWYRQAPGKQREFVAGFTSAGS | 37 |
| | TNYGDSVKGRFTISRDNAKNTVYLQMDSLIPEDTAVYYCHYLGDWGQGTQVTVSS | |
| KB_B09- | DSAFX₂₈MNT | 38 |
| KB_B10 | | |
| CDR1 | | |
| KB_B09- | IVSDDNT | 39 |
| KB_B10 | | |
| CDR2 | | |
| KB_B09- | KGDVV | 40 |
| KB_B10 | | |
| CDR3 | | |
| KB_B09 | DSAFSMNT | 41 |
| CDR1 | | |
| KB_B10 | DSAFNMNT | 42 |
| CDR1 | | |
| KB_B09- | QVQLQESGGGLVQVGGSLRLSCAAS | 43 |
| KB_B10 | | |
| FR1 | | |
| KB_B09- | MYWYRQAPGKX29REFVAX₃₀ | 44 |
| KB_B10 | | |
| FR2 | | |
| KB_B09- | X₃₁YADSVKGRFTISRDNAKNTVYLQMNX₃₂LKPEDTAX₃₃YYC | 45 |
| KB_B10 | | |
| FR3 | | |
| KB_B09 | QVQLQESGGGLVQVGGSLRLSCAASDSAFSMNTMYWYRQAPGKQREFVAYIVSDDN | 46 |
| | TRYADSVKGRFTISRDNAKNTVYLQMNSLKPEDTAVYYCKGDVVWGQGTQVTVSS | |
| KB_B10 | QVQLQESGGGLVQVGGSLRLSCAASDSAFNMNTMYWYRQAPGKSREFVAWIVSDDN | 47 |
| | TQYADSVKGRFTISRDNAKNTVYLQMNNLKPEDTGVYYCKGDVVWGQGTQVTVSS | |
| His-tagged | AHHHHHHSGRLYWDDLKRKLSEKLDSTDFTGTIKLLNENSYVPREAGSQKDENLAL | 48 |
| ectodomain | YVENQFREFKLSKVWRDQHFVKIQVKDSAQNSVIIVDKNGRLVYLVENPGGYVAYS | |
| of hTfR1 | KAATVTGKLVHANFGTKKDFEDLYTPVNGSIVIVRAGKITFAEKVANAESLNAIGV | |
| | LIYMDQTKFPIVNAELSFFGHAHLGTGDPYTPGFPSFNHTQFPPSRSSGLPNIPVQ | |
| | TISRAAAEKLFGNMEGDCPSDWKTDSTCRMVTSESKNVKLTVSNVLKEIKILNIFG | |
| | VIKGFVEPDHYVVVGAQRDAWGPGAAKSGVGTALLLKLAQMFSDMVLKDGFQPSRS | |
| | IIFASWSAGDFGSVGATEWLEGYLSSLHLKAFTYINLDKAVLGTSNEKVSASPLLY | |
| | TLIEKTMQNVKHPVTGQFLYQDSNWASKVEKLTLDNAAFPFLAYSGIPAVSFCFCE | |
| | DTDYPYLGTTMDTYKELIERIPELNKVARAAAEVAGQFVIKLTHDVELNLDYERYN | |
| | SQLLSFVRDLNQYRADIKEMGLSLQWLYSARGDFFRATSRLTTDFGNAEKTDRFVM | |
| | KKLNDRVMRVEYHELSPYVSPKESPFRHVFWGSGSHTLPALLENLKLRKQNNGAFN | |
| | ETLFRNQLALATWTIQGAANALSGDVWDIDNEF | |
| His-tagged | AHHHHHHGSRLYWADLKTLLSEKLNSIEFADTIKQLSQNTYTPREAGSQKDESLAY | 49 |
| ectodomain | YIENQFHEFKFSKVWRDEHYVKIQVKSSIGQNMVTIVQSNGNLDPVESPEGYVAFS | |
| of mTfR1 | KPTEVSGKLVHANFGTKKDFEELSYSVNGSLVIVRAGEITFAEKVANAQSFNAIGV | |
| | LIYMDKNKFPVVEADLALFGHAHLGTGDPYTPGFPSFNHTQFPPSQSSGLPNIPVQ | |
| | TISRAAAEKLFGKMEGSCPARWNIDSSCKLELSQNQNVKLIVKNVLKERRILNIFG | |
| | VIKGYEEPDRYVVVGAQRDALGAGVAAKSSVGTGLLLKLAQVESDMISKDGFRPSR | |
| | SIIFASWTAGDFGAVGATEWLEGYLSSLHLKAFTYINLDKVVLGTSNFKVSASPLL | |
| | YTLMGKIMQDVKHPVDGKSLYRDSNWISKVEKLSFDNAAYPFLAYSGIPAVSFCFC | |
| | EDADYPYLGTRLDTYEALTQKVPQLNQMVRTAAEVAGQLIIKLTHDVELNLDYEMY | |
| | NSKLLSFMKDLNQFKTDIRDMGLSLQWLYSARGDYFRATSRLTTDFHNAEKTNRFV | |
| | MREINDRIMKVEYHFLSPYVSPRESPFRHIFWGSGSHTLSALVENLKLRQKNITAF | |
| | NETLFRNQLALATWTIQGVANALSGDIWNIDNEF | |
| His-tagged | AHHHHHHSGRLYWDDLKRKLSEKLDTTDFTSTIKLLNENLYVPREAGSQKDENLAL | 50 |
| ectodomain | YIENQFREFKLSKVWRDQHFVKIQVKDSAQNSVIIVDKNGGLVYLVENPGGYVAYS | |
| of cTfR1 | KAATVTGKLVHANFGTKKDFEDLDSPVNGSIVIVRAGKITFAEKVANAESLNAIGV | |
| | LIYMDQTKFPIVKADLSFFGHAHLGTGDPYTPGFPSFNHTQFPPSQSSGLPNIPVQ | |
| | TISRAAAEKLFGNMEGDCPSDWKTDSTCKMVTSENKSVKLTVSNVLKETKILNIFG | |
| | VIKGFVEPDHYVVVGAQRDAWGPGAAKSSVGTALLLKLAQMFSDMVLKDGFQPSRS | |
| | IIFASWSAGDFGSVGATEWLEGYLSSLHLKAFTYINLDKAVLGTSNFKVSASPLLY | |
| | TLIEKTMQDVKHPVTGRSLYQDSNWASKVEKLTLDNAAFPFLAYSGIPAVSFCFCE | |
| | DTDYPYLGTTMDTYKELVERIPELNKVARAAAEVAGQFVIKLTHDTELNLDYERYN | |
| | SQLLLFLRDLNQYRADVKEMGLSLQWLYSARGDFFRATSRLTTDERNAEKRDKFVM | |
| | KKLNDRVMRVEYYFLSPYVSPKESPFRHVFWGSGSHTLSALLESLKLRRQNNSAFN | |
| | ETLFRNQLALATWTIQGAANALSGDVWDIDNEF | |
| N-terminal | MDWLRNLLFLMAAAQSINA | 51 |
| signal | | |
| peptide | | |
| CALL001 | GTCCTGGCTGCTCTTCTACAAGG | 52 |
| CALL002 | GGTACGTGCTGTTGAACTGTTCC | 53 |
| VHH-R-SfiI | AAAGGCCCAGCCGGCCATGGCGCAGGTGCAGCTGCAGGAGTCTGGRGGAGG | 54 |
| VHH-F-SfiI | AAAGGCCTCCCGGGCCACGTTTTGAGGAGACGGTGACCTGGGT | 55 |
| 6×His | HHHHHH | 56 |
| C-tag | EPEA | 57 |
| Human | DKTHTCPPCPAPELLGGPSVFLFPPKPKDTLMISRTPEVTCVVVDVSHEDPEVKEN | 58 |
| IgG1 Fc | WYVDGVEVHNAKTKPREEQYNSTYRVVSVLTVLHQDWLNGKEYKCKVSNKALPAPI | |
| | EKTISKAKGQPREPQVYTLPPSRDELTKNQVSLTCLVKGFYPSDIAVEWESNGQPE | |
| | NNYKTTPPVLDSDGSFFLYSKLTVDKSRWQQGNVFSCSVMHEALHNHYTQKSLSLS | |
| | PGK | |
| KB_B01 Fc | QVQLQESGGGLVQAGGSLRLSCAASGTPFSNNYMRWYRQAPGKQREWVAGFTSGGS | 59 |
| fusion | TNYADSVKGRFTISRDNAEGTVYLQMDNLKPEDTAVYYCYFLDVWGQGTQVTVSSG | |
| | GDKTHTCPPCPAPELLGGPSVFLFPPKPKDTLMISRTPEVTCVVVDVSHEDPEVKE | |
| | NWYVDGVEVHNAKTKPREEQYNSTYRVVSVLTVLHQDWLNGKEYKCKVSNKALPAP | |
| | IEKTISKAKGQPREPQVYTLPPSRDELTKNQVSLTCLVKGFYPSDIAVEWESNGQP | |
| | ENNYKTTPPVLDSDGSFFLYSKLTVDKSRWQQGNVFSCSVMHEALHNHYTQKSLSL | |
| | SPGK | |
| KB_B02 Fc | QVQLQESGGGLVQAGGSLRLSCTASGTPFTNNYMRWYRQAPGKQREWVAGFTSAGN | 60 |
| fusion | TNYADSVKGRFTISRDNAEGTVYLQMDNLKPEDTAVYYCYFLDVWGQGTQVTVSSG | |
| | GDKTHTCPPCPAPELLGGPSVFLFPPKPKDTLMISRTPEVTCVVVDVSHEDPEVKF | |
| | NWYVDGVEVHNAKTKPREEQYNSTYRVVSVLTVLHQDWLNGKEYKCKVSNKALPAP | |
| | IEKTISKAKGQPREPQVYTLPPSRDELTKNQVSLTCLVKGFYPSDIAVEWESNGQP | |
| | ENNYKTTPPVLDSDGSFFLYSKLTVDKSRWQQGNVFSCSVMHEALHNHYTQKSLSL | |
| | SPGK | |
| KB_B03 Fc | QVQLQESGGGLVQAGGSLRLSCAASGTDESLNYMRWYRQAPGKQREWVAGFTTHGD | 61 |
| fusion | TNYADSMKGRFTISRDNAKNTVYLQMDSLKPEDTAVYFCYFLDNWGQGTQVTVSSG | |
| | GDKTHTCPPCPAPELLGGPSVFLFPPKPKDTLMISRTPEVTCVVVDVSHEDPEVKF | |
| | NWYVDGVEVHNAKTKPREEQYNSTYRVVSVLTVLHQDWLNGKEYKCKVSNKALPAP | |
| | IEKTISKAKGQPREPQVYTLPPSRDELTKNQVSLTCLVKGFYPSDIAVEWESNGQP | |
| | ENNYKTTPPVLDSDGSFFLYSKLTVDKSRWQQGNVFSCSVMHEALHNHYTQKSLSL | |
| | SPGK | |
| KB_B04 Fc | QVQLQESGGGLVQAGGSLRLSCALSGTDESMNYMRWYRQAPGKQREWVAGFTSSGN | 62 |
| fusion | TNYPDSVKGRFTISRDNAKNTVYLQMDSLKPEDTAVYYCYFLDNWGQGTQVTVSSG | |
| | GDKTHTCPPCPAPELLGGPSVFLFPPKPKDTLMISRTPEVTCVVVDVSHEDPEVKE | |
| | NWYVDGVEVHNAKTKPREEQYNSTYRVVSVLTVLHQDWLNGKEYKCKVSNKALPAP | |
| | IEKTISKAKGQPREPQVYTLPPSRDELTKNQVSLTCLVKGFYPSDIAVEWESNGQP | |
| | ENNYKTTPPVLDSDGSFFLYSKLTVDKSRWQQGNVFSCSVMHEALHNHYTQKSLSL | |
| | SPGK | |
| KB_B05 Fc | QVQLQESGGGLVQAGGSLRLSCVASGTDESMNYMRWYRQAPGKQREWVAGFTSTGS | 63 |
| fusion | TNYPDSMKGRFTISRDNAKNTVYLQMDSLKPEDTAVYYCHFLGDWGQGTQVTVSSG | |
| | GDKTHTCPPCPAPELLGGPSVFLFPPKPKDTLMISRTPEVTCVVVDVSHEDPEVKF | |
| | NWYVDGVEVHNAKTKPREEQYNSTYRVVSVLTVLHQDWLNGKEYKCKVSNKALPAP | |
| | IEKTISKAKGQPREPQVYTLPPSRDELTKNQVSLTCLVKGFYPSDIAVEWESNGQP | |
| | ENNYKTTPPVLDSDGSFFLYSKLTVDKSRWQQGNVFSCSVMHEALHNHYTQKSLSL | |
| | SPGK | |
| KB_B06 Fc | QVQLQESGGGLVQTGGSLRLSCVVSGTDFSLNYLRWYRQAPGNQREWVAGFTATGS | 64 |
| fusion | TNYPDSMKGRFTIGRDNAKNTVYLQMDSLKPEDTAVYYCHFLGDWGQGTQVTVSSG | |
| | GDKTHTCPPCPAPELLGGPSVFLFPPKPKDTLMISRTPEVTCVVVDVSHEDPEVKF | |
| | NWYVDGVEVHNAKTKPREEQYNSTYRVVSVLTVLHQDWLNGKEYKCKVSNKALPAP | |
| | IEKTISKAKGQPREPQVYTLPPSRDELTKNQVSLTCLVKGFYPSDIAVEWESNGQP | |
| | ENNYKTTPPVLDSDGSFFLYSKLTVDKSRWQQGNVFSCSVMHEALHNHYTQKSLSL | |
| | SPGK | |
| KB_B07 Fc | QVQLQESGGGLVQTGGSLRLSCVVSGTDFSLNYLRWYRQAPGKQREWVAGFTASGS | 65 |
| fusion | TNYPDSMKGRFTIGRDNAKNTVYLQMDSLTPEDTAVYYCHFLGDWGQGTQVTVSSG | |
| | GDKTHTCPPCPAPELLGGPSVFLFPPKPKDTLMISRTPEVTCVVVDVSHEDPEVKF | |
| | NWYVDGVEVHNAKTKPREEQYNSTYRVVSVLTVLHQDWLNGKEYKCKVSNKALPAP | |
| | IEKTISKAKGQPREPQVYTLPPSRDELTKNQVSLTCLVKGFYPSDIAVEWESNGQP | |
| | ENNYKTTPPVLDSDGSFFLYSKLTVDKSRWQQGNVFSCSVMHEALHNHYTQKSLSL | |
| | SPGK | |
| KB_B08 Fc | QVQLQESGRGVVQAGGSLRLSCAASGNRFGIEYMRWYRQAPGKQREFVAGFTSAGS | 66 |
| fusion | TNYGDSVKGRFTISRDNAKNTVYLQMDSLIPEDTAVYYCHYLGDWGQGTQVTVSSG | |
| | GDKTHTCPPCPAPELLGGPSVFLFPPKPKDTLMISRTPEVTCVVVDVSHEDPEVKF | |
| | NWYVDGVEVHNAKTKPREEQYNSTYRVVSVLTVLHQDWLNGKEYKCKVSNKALPAP | |
| | IEKTISKAKGQPREPQVYTLPPSRDELTKNQVSLTCLVKGFYPSDIAVEWESNGQP | |
| | ENNYKTTPPVLDSDGSFFLYSKLTVDKSRWQQGNVFSCSVMHEALHNHYTQKSLSL | |
| | SPGK | |
| KB_B09 Fc | QVQLQESGGGLVQVGGSLRLSCAASDSAFSMNTMYWYRQAPGKQREFVAYIVSDDN | 67 |
| fusion | TRYADSVKGRFTISRDNAKNTVYLQMNSLKPEDTAVYYCKGDVVWGQGTQVTVSSG | |
| | GDKTHTCPPCPAPELLGGPSVFLFPPKPKDTLMISRTPEVTCVVVDVSHEDPEVKF | |
| | NWYVDGVEVHNAKTKPREEQYNSTYRVVSVLTVLHQDWLNGKEYKCKVSNKALPAP | |
| | IEKTISKAKGQPREPQVYTLPPSRDELTKNQVSLTCLVKGFYPSDIAVEWESNGQP | |
| | ENNYKTTPPVLDSDGSFFLYSKLTVDKSRWQQGNVFSCSVMHEALHNHYTQKSLSL | |
| | SPGK | |
| 3D6 VH | EVKLVESGGGLVKPGASLKLSCAASGFTFSNYGMSWVRQNSDKRLEWVASIRSGGG | 68 |
| | RTYYSDNVKGRFTISRENAKNTLYLQMSSLKSEDTALYYCVRYDHYSGSSDYWGQG | |
| | TTVTVS | |
| 3D6 VL | YVVMTQTPLTLSVTIGQPASISCKSSQSLLDSDGKTYLNWLLQRPGQSPKRLIYLV | 69 |
| | SKLDSGVPDRFTGSGSGTDFTLKISRIEAEDLGLYYCWQGTHFPRTFGGGTKLEIK | |
| 3×(G4S) | GGGGSGGGGSGGGGS | 70 |
| linker | | |
| KB_B01- | QVQLQESGGGLVQAGGSLRLSCAASGTPFSNNYMRWYRQAPGKQREWVAGFTSGGS | 71 |
| scFv | TNYADSVKGRFTISRDNAEGTVYLQMDNLKPEDTAVYYCYFLDVWGQGTQVTVSSG | |
| | GGGSGGGGSGGGGSEVKLVESGGGLVKPGASLKLSCAASGFTFSNYGMSWVRQNSD | |
| | KRLEWVASIRSGGGRTYYSDNVKGRFTISRENAKNTLYLQMSSLKSEDTALYYCVR | |
| | YDHYSGSSDYWGQGTTVTVSGGGGSGGGGGGGGSYVVMTQTPLTLSVTIGQPASI | |
| | SCKSSQSLLDSDGKTYLNWLLQRPGQSPKRLIYLVSKLDSGVPDRFTGSGSGTDFT | |
| | LKISRIEAEDLGLYYCWQGTHFPRTFGGGTKLEIKGSENLYFQSHHHHHH | |
| scFv- | EVKLVESGGGLVKPGASLKLSCAASGFTFSNYGMSWVRQNSDKRLEWVASIRSGGG | 72 |
| KB_B01 | RTYYSDNVKGRFTISRENAKNTLYLQMSSLKSEDTALYYCVRYDHYSGSSDYWGQG | |
| | TTVTVSGGGGSGGGGSGGGGSYVVMTQTPLTLSVTIGQPASISCKSSQSLLDSDGK | |
| | TYLNWLLQRPGQSPKRLIYLVSKLDSGVPDRFTGSGSGTDFTLKISRIEAEDLGLY | |
| | YCWQGTHFPRTFGGGTKLEIKGGGGSGGGGSGGGGSQVQLQESGGGLVQAGGSLRL | |
| | SCAASGTPFSNNYMRWYRQAPGKQREWVAGFTSGGSTNYADSVKGRFTISRDNAEG | |
| | TVYLQMDNLKPEDTAVYYCYFLDVWGQGTQVTVSSGSENLYFQSHHHHHH | |
| BV-scFv | EVQLVESGGGVVQPGGSLKLSCVASGTDESINFIRWYRQAPGKQREFVAGFTATGN | 73 |
| | TNYADSMKGRFTISRDNTKNAVYLQIDSLKPEDTAVYYCYMLDKWGQGTQVTVSSG | |
| | GGGSGGGGSGGGGSEVKLVESGGGLVKPGASLKLSCAASGFTFSNYGMSWVRQNSD | |
| | KRLEWVASIRSGGGRTYYSDNVKGRFTISRENAKNTLYLQMSSLKSEDTALYYCVR | |
| | YDHYSGSSDYWGQGTTVTVSGGGGSGGGGSGGGGSYVVMTQTPLTLSVTIGQPASI | |
| | SCKSSQSLLDSDGKTYLNWLLQRPGQSPKRLIYLVSKLDSGVPDRFTGSGSGTDFT | |
| | LKISRIEAEDLGLYYCWQGTHFPRTFGGGTKLEIKGSENLYFQSHHHHHH | |
| scFv-BV | EVKLVESGGGLVKPGASLKLSCAASGFTFSNYGMSWVRQNSDKRLEWVASIRSGGG | 74 |
| | RTYYSDNVKGRFTISRENAKNTLYLQMSSLKSEDTALYYCVRYDHYSGSSDYWGQG | |
| | TTVTVSGGGGSGGGGSGGGGSYVVMTQTPLTLSVTIGQPASISCKSSQSLLDSDGK | |
| | TYLNWLLQRPGQSPKRLIYLVSKLDSGVPDRFTGSGSGTDFTLKISRIEAEDLGLY | |
| | YCWQGTHFPRTFGGGTKLEIKGGGGSGGGGSGGGGSEVQLVESGGGVVQPGGSLKL | |
| | SCVASGTDESINFIRWYRQAPGKQREFVAGFTATGNTNYADSMKGRFTISRDNTKN | |
| | AVYLQIDSLKPEDTAVYYCYMLDKWGQGTQVTVSSGSENLYFQSHHHHHH | |
| BA1 | EVQLVQSGAEVKKPGESLKISCKGSGYSFTNYWLGWVRQMPGKGLEWMGDIYPGGD | 75 |
| | YPTYSEKFKVQVTISADKSISTAYLQWSSLKASDTAMYYCARSGNYDEVAYWGQGT | |
| | LVTVSSASTKGPSVFPLAPSSKSTSGGTAALGCLVKDYFPEPVTVSWNSGALTSGV | |
| | HTFPAVLQSSGLYSLSSVVTVPSSSLGTQTYICNVNHKPSNTKVDKKVEPKSCDKT | |
| | HTCPPCPAPELLGGPSVFLFPPKPKDTLMISRTPEVTCVVVDVSHEDPEVKENWYV | |
| | DGVEVHNAKTKPREEQYNSTYRVVSVLTVLHQDWLNGKEYKCKVSNKALPAPIEKT | |
| | ISKAKGQPREPQVYTLPPSRDELTKNQVSLTCLVKGFYPSDIAVEWESNGQPENNY | |
| | KTTPPVLDSDGSFFLYSKLTVDKSRWQQGNVFSCSVMHEALHNHYTQKSLSLSPGK | |
| | DIVMTQTPLSLSVTPGQPASISCRSSQSLVHSNGNTYLHWYLQKPGQSPQLLIYKV | |
| | SNRFSGVPDRFSGSGSGTDFTLKISRVEAEDVGVYYCSQSTHVPWTFGQGTKVEIK | |
| | RTVAAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSENRGEC | |
| BA2 | EVQLQQSGPELVKPGASMKISCKASGYSFTGYTMNWVKQSHGENLEWIGRINPHNG | 76 |
| | GTDYNQKFKDKAPLTVDKSSNTAYMELLSLTSEDSAVYYCARGYYYYSLDYWGQGT | |
| | SVTVSSASTKGPSVFPLAPSSKSTSGGTAALGCLVKDYFPEPVTVSWNSGALTSGV | |
| | HTFPAVLQSSGLYSLSSVVTVPSSSLGTQTYICNVNHKPSNTKVDKKVEPKSCDKT | |
| | HTCPPCPAPELLGGPSVFLFPPKPKDTLMISRTPEVTCVVVDVSHEDPEVKENWYV | |
| | DGVEVHNAKTKPREEQYNSTYRVVSVLTVLHQDWLNGKEYKCKVSNKALPAPIEKT | |
| | ISKAKGQPREPQVYTLPPSRDELTKNQVSLTCLVKGFYPSDIAVEWESNGQPENNY | |
| | KTTPPVLDSDGSFFLYSKLTVDKSRWQQGNVFSCSVMHEALHNHYTQKSLSLSPGK | |
| | QIVLTQSPAIMSASPGEKVTMTCSASSSIDYIHWYQQKSGTSPKRWIYDTSKLASG | |
| | VPARFSGSGSGTSYSLTISSMEPEDAATYYCHQRNSYPWTFGGGTRLEIRRTVAAP | |
| | SVFIFPPSDEQLKSGTASVVCLLNNFYPREAKVQWKVDNALQSGNSQESVTEQDSK | |
| | DSTYSLSSTLTLSKADYEKHKVYACEVTHQGLSSPVTKSENRGEC | |
| FR1 | QVQLQESGGGLVQX₁₆GGSLRLSCX₁₇X₁₈S | 77 |
| consensus | | |
| 2 | | |
| FR2 | X₁₉RWYRQAPGX₂₀QREWVAG | 78 |
| consensus | | |
| 2 | | |
| Mouse | MGWSCIILFLVATATGVHS | 79 |
| heavy chain | | |
| signal | | |
| peptide | | |
| VHH- | EVQLVESGGGVVQPGGSLKLSCVASGTDESINFIRWYRQAPGKQREFVAGFTATGN | 80 |
| antibody in | TNYADSMKGRFTISRDNTKNAVYLQIDSLKPEDTAVYYCYMLDKWGQGTQVTVSS | |
| BV | | |
| murine IgG | METDTLLLWVLLLWVPGST | 81 |
| kappa light | | |
| chain signal | | |
| peptide | | |
| KB_B10 Fc | QVQLQESGGGLVQVGGSLRLSCAASDSAFNMNTMYWYRQAPGKSREFVAWIVSDDN | 82 |
| fusion | TQYADSVKGRFTISRDNAKNTVYLQMNNLKPEDTGVYYCKGDVVWGQGTQVTVSSG | |
| | GDKTHTCPPCPAPELLGGPSVFLFPPKPKDTLMISRTPEVTCVVVDVSHEDPEVKE | |
| | NWYVDGVEVHNAKTKPREEQYNSTYRVVSVLTVLHQDWLNGKEYKCKVSNKALPAP | |
| | IEKTISKAKGQPREPQVYTLPPSRDELTKNQVSLTCLVKGFYPSDIAVEWESNGQP | |
| | ENNYKTTPPVLDSDGSFFLYSKLTVDKSRWQQGNVFSCSVMHEALHNHYTQKSLSL | |
| | SPGK | |
| KB_B11 | QVQLQESGGGLVQAGGSLRLSCAASGTPFSNNYMRWYRQAPGKQREWVAGFTSGGS | 83 |
| | TNYADSVKGRFTISRDNAKGTVYLQMNSLKPEDTAVYYCYFLDVWGQGTQVTVSS | |
| KB_B12 | QVQLQESGGGLVQAGGSLRLSCAASGTDFSLNYMRWYRQAPGKQREWVAGFTTHGD | 84 |
| | TNYADSMKGRFTISRDNAKNTVYLQMNSLKPEDTAVYFCYFLDNWGQGTQVTVSS | |
| KB_B12- | QVQLQESGGGLVQAGGSLRLSCAASGTDESLNYMRWYRQAPGKQREWVAGFTTHGD | |
| scFv | TNYADSMKGRFTISRDNAKNTVYLQMNSLKPEDTAVYFCYFLDNWGQGTQVTVSSG | 85 |
| | GGGSGGGGSGGGGSEVKLVESGGGLVKPGASLKLSCAASGFTFSNYGMSWVRQNSD | |
| | KRLEWVASIRSGGGRTYYSDNVKGRFTISRENAKNTLYLQMSSLKSEDTALYYCVR | |
| | YDHYSGSSDYWGQGTTVTVSGGGGSGGGGSGGGGSYVVMTQTPLTLSVTIGQPASI | |
| | SCKSSQSLLDSDGKTYLNWLLQRPGQSPKRLIYLVSKLDSGVPDRFTGSGSGTDFT | |
| | LKISRIEAEDLGLYYCWQGTHFPRTFGGGTKLEIK | |
| His-tag | GGHHHHHH | 86 |
| ProA | QVQLQESGGGLVQPGGSLRLSCAASGRTFSSYAMGWFRQAPGKQREFVAAIRWSGG | |
| positive | YTYYTDSVKGRFTISRDNAKTTVYLQMNSLKPEDTAVYYCAATYLSSDYSRYALPQ | 87 |
| control | RPLDYDYWGQGTQVTVSSLE | |

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1.-39. (canceled)

40. A variable domain of heavy chain-only, VHH, antibody binding specifically to a transferrin receptor 1, TfR1, comprising:
a complementarity determining region 1, CDR1, consisting of the amino acid sequence GX₁X₂FX₃X₄X₅Y, wherein X₁ is T or N, X₂ is P, D or R, X₃ is S, T or G, X₄ is N, L, M or I, and X₅ is N or E;
a CDR2 consisting of the amino acid sequence FTX₆X₇GX₈T as defined in SEQ ID NO: 1, wherein X₆ is S, T or A, X₇ is G, A, H, S or T, and X₈ is S, N or D; and
a CDR3 consisting of the amino acid sequence X₉X₁₀LX₁₁X₁₂, wherein X₉ is Y or H, X₁₀ is F or Y, X₁₁ is D or G, and X₁₂ is V, N or D.

41. The VHH antibody according to claim 40, wherein
the CDR1 consists of the amino acid sequence GTX₂FX₃X₄NY as defined in SEQ ID NO: 2, wherein X₂ is P or D, X₃ is S or T, and X₄ is N, L or M; and
the CDR3 consists of the amino acid sequence X₉FLX₁₁X₁₂, wherein X₉ is Y or H, X₁₁ is D or G, and X₁₂ is V, N or D.

42. The VHH antibody according to claim 41, wherein
the CDR1 consists of the amino acid sequence GTDFSX₄NY as defined in SEQ ID NO: 3, wherein X₄ is L or M;
the CDR2 consists of the amino acid sequence FTX₆X₇GST as defined in SEQ ID NO: 4, wherein X₆ is S or A, and X₇ is S or T; and
the CDR3 consists of the amino acid sequence HFLGD as defined in SEQ ID NO: 5.

43. The VHH antibody according to claim 42, wherein
the CDR1 consists of the amino acid sequence GTDFSLNY as defined in SEQ ID NO: 6;
the CDR2 consists of the amino acid sequence FTAX₇GST as defined in SEQ ID NO: 7, wherein X₇ is S or T; and
the CDR3 consists of the amino acid sequence HFLGD as defined in SEQ ID NO: 5.

44. The VHH antibody according to claim 43, wherein
the CDR1 consists of the amino acid sequence GTDFSLNY as defined in SEQ ID NO: 6;
the CDR2 consists of the amino acid sequence FTATGST as defined in SEQ ID NO: 8; and
the CDR3 consists of the amino acid sequence HFLGD as defined in SEQ ID NO: 5.

45. The VHH antibody according to claim 43, wherein
the CDR1 consists of the amino acid sequence GTDFSLNY as defined in SEQ ID NO: 6;
the CDR2 consists of the amino acid sequence FTASGST as defined in SEQ ID NO: 9; and
the CDR3 consists of the amino acid sequence HFLGD as defined in SEQ ID NO: 5.

46. The VHH antibody according to claim 42, wherein
the CDR1 consists of the amino acid sequence GTDFSMNY as defined in SEQ ID NO: 10;
the CDR2 consists of the amino acid sequence FTSTGST as defined in SEQ ID NO: 11; and
the CDR3 consists of the amino acid sequence HFLGD as defined in SEQ ID NO: 5.

47. The VHH antibody according to claim 41, wherein
the CDR1 consists of the amino acid sequence GTX₂FX₃X₄NY as defined in SEQ ID NO: 2, wherein X₂ is P or D, X₃ is S or T, and X₄ is N, L or M;
the CDR2 consists of the amino acid sequence FTX₆X₇GX₈T as defined in SEQ ID NO: 1, wherein X₆ is S or T, X₇ is G, A, H or S, and X₈ is S, N or D; and
the CDR3 consists of the amino acid sequence YFLDX₁₂ as defined in SEQ ID NO: 12, wherein X₁₂ is V or N.

48. The VHH antibody according to claim 47, wherein
the CDR1 consists of the amino acid sequence GTPFX₃NNY as defined in SEQ ID NO: 13, wherein X₃ is S or T;
the CDR2 consists of the amino acid sequence FTSX₇GX₈T as defined in SEQ ID NO: 14, wherein X₇ is G or A, and X₈ is S or N; and
the CDR3 consists of the amino acid sequence YFLDV as defined in SEQ ID NO: 15.

49. The VHH antibody according to claim 48, wherein
the CDR1 consists of the amino acid sequence GTPFSNNY as defined in SEQ ID NO: 16;
the CDR2 consists of the amino acid sequence FTSGGST as defined in SEQ ID NO: 17; and
the CDR3 consists of the amino acid sequence YFLDV as defined in SEQ ID NO: 15.

50. The VHH antibody according to claim 48, wherein
the CDR1 consists of the amino acid sequence GTPFTNNY as defined in SEQ ID NO: 18;
the CDR2 consists of the amino acid sequence FTSAGNT as defined in SEQ ID NO: 19; and
the CDR3 consists of the amino acid sequence YFLDV as defined in SEQ ID NO: 15.

51. The VHH antibody according to claim 47, wherein
the CDR1 consists of the amino acid sequence GTDFSX₄NY as defined in SEQ ID NO: 3, wherein X₄ is L or M;
the CDR2 consists of the amino acid sequence FTX₆X₇GX₈T as defined in SEQ ID NO: 1, wherein X₆ is S or T, X₇ is H or S, and X₈ is N or D; and
the CDR3 consists of the amino acid sequence YFLDN as defined in SEQ ID NO: 20.

52. The VHH antibody according to claim 51, wherein
the CDR1 consists of the amino acid sequence GTDFSLNY as defined in SEQ ID NO: 6;
the CDR2 consists of the amino acid sequence FTTHGDT as defined in SEQ ID NO: 21; and
the CDR3 consists of the amino acid sequence YFLDN as defined in SEQ ID NO: 20.

53. The VHH antibody according to claim 51, wherein
the CDR1 consists of the amino acid sequence GTDFSMNY as defined in SEQ ID NO: 10;
the CDR2 consists of the amino acid sequence FTSSGNT as defined in SEQ ID NO: 22; and
the CDR3 consists of the amino acid sequence YFLDN as defined in SEQ ID NO: 20.

54. The VHH antibody according to claim 40, wherein
the CDR1 consists of the amino acid sequence GNRFGIEY as defined in SEQ ID NO: 23;
the CDR2 consists of the amino acid sequence FTSAGST as defined in SEQ ID NO: 24; and
the CDR3 consists of the amino acid sequence HYLGD as defined in SEQ ID NO: 25.

55. The VHH antibody according to claim 40, wherein
the VHH antibody is of formula: framework region 1, FR1, -CDR1-FR2-CDR2-FR3-CDR3-FR4;
FR1 has an amino acid sequence QVQLQESGX₁₄GX₁₅VQX₁₆GGSLRLSCX₁₇X₁₈S as defined in SEQ ID NO: 26, wherein X₁₄ is R or G, X₁₅ is L or V, X₁₆ is A, or T, X₁₇ is A, T or V, and X₁₈ is A, L or V;
FR2 has an amino acid sequence X₁₉RWYRQAPGX₂₀QREX₂₁VAG as defined in SEQ ID NO: 27, wherein X₁₉ is M or L, X₂₀ is K or N, and X₂₁ is W or F;
FR3 has an amino acid sequence NYX₂₀DSX₂₁KGRFTIX₂₂RDNAX₂₃X₂₄TVYLQMDX₂₅LX₂₆PEDTAVYX₂₇C as defined in SEQ ID NO: 28, wherein X₂₀ is A, P or G, X₂₁ is V or M, X₂₂ is S or G, X₂₃ is E or K, X₂₄ is G or N, X₂₅ is N or S, X₂₆ is K, I or T, and X₂₇ is Y or F; and
FR4 has an amino acid sequence WGQGTQVTVSS as defined in SEQ ID NO: 29.

56. The VHH antibody according to claim 40, wherein the VHH antibody has an amino acid sequence selected from the group consisting of SEQ ID NO: 30 to 37.

57. A variable domain of heavy chain-only, VHH, antibody binding specifically to a transferrin receptor 1, TfR1, comprising:
a complementarity determining region 1, CDR1, consisting of the amino acid sequence DSAFX₂₈MNT as defined in SEQ ID NO: 38, wherein X₂₈ is S or N;
a CDR2 consisting of the amino acid sequence IVSDDNT as defined in SEQ ID NO: 39; and
a CDR3 consisting of the amino acid sequence KGDVV as defined in SEQ ID NO: 40.

58. The VHH antibody according to claim 57, wherein
the CDR1 consists of the amino acid sequence DSAFSMNT as defined in SEQ ID NO: 41;
the CDR2 consists of the amino acid sequence IVSDDNT as defined in SEQ ID NO: 39; and
the CDR3 consists of the amino acid sequence KGDVV as defined in SEQ ID NO: 40.

59. The VHH antibody according to claim 57, wherein
the CDR1 consists of the amino acid sequence DSAFNMNT as defined in SEQ ID NO: 42;
the CDR2 consists of the amino acid sequence IVSDDNT as defined in SEQ ID NO: 39; and
the CDR3 consists of the amino acid sequence KGDVV as defined in SEQ ID NO: 40.

60. The VHH antibody according to claim 57, wherein
the VHH antibody is of formula: framework region 1, FR1, -CDR1-FR2-CDR2-FR3-CDR3-FR4;
FR1 has an amino acid sequence QVQLQESGGGLVQVGGSLRLSCAAS as defined in SEQ ID NO: 43;
FR2 has an amino acid sequence MYWYRQAPGKX₂₉REFVAX₃₀ as defined in SEQ ID NO: 44, wherein X₂₉ is Q or S, and X₃₀ is Y or W;
FR3 has an amino acid sequence X₃₁YADSVKGRFTISRDNAKNTVYLQMNX₃₂LKPEDTAX₃₃YYC as defined in SEQ ID NO: 45, wherein X₃₁ is R or Q, X₃₂ is S or N, and X₃₃ is V or G; and
FR4 has an amino acid sequence WGQGTQVTVSS as defined in SEQ ID NO: 29.

61. The VHH antibody according to claim 57, wherein the VHH antibody has an amino acid sequence selected from the group consisting of SEQ ID NO: 46 to 47.

62. The VHH antibody according to claim 40 or 57, wherein the VHH antibody binds specifically to human TfR1.

63. The VHH antibody according to claim 40 or 57, wherein the VHH antibody binds specifically to mouse TfR1.

64. The VHH antibody according to claim 40 or 57, wherein the VHH antibody binds specifically to cynomolgus TRI.

65. The VHH antibody according to claim 40 or 57, wherein the VHH antibody is a camelid VHH antibody.

66. The VHH antibody according to claim 40 or 57, wherein the VHH antibody is a humanized VHH antibody.

67. The VHH antibody according to claim 40 or 57, wherein the VHH antibody binds to protein A.

68. A fusion molecule comprising a VHH antibody according to claim 40 or 57 linked to at least one molecule.

69. The fusion molecule according to claim 68, wherein the fusion protein comprises the VHH antibody according to claim 40 or 57 covalently linked to the at least one molecule through a linker.

70. The fusion molecule according to claim 69, wherein the at least one molecule is selected from the group consisting of a therapeutic agent and an imaging agent.

71. The fusion molecule according to claim 70, wherein the therapeutic agent is selected from the group consisting of a therapeutic agent capable of treating a central nervous system disease or disorder, a therapeutic agent is capable of treating cancer, and a therapeutic agent is capable of treating muscular dystrophy.

72. The fusion molecule according to claim 70, wherein the imaging agent is selected from the group consisting of a position emission tomography tracer, a single-photon emission computerized tomography tracer, a fluorescent probe, a luminescent probe, a metal complex containing probe, and a near infrared fluorescent probe.

73. A pharmaceutical composition comprising:
a fusion molecule according to claim 68, wherein the at least one molecule is a therapeutic agent; and
a pharmaceutically acceptable vehicle.

74. A nucleic acid molecule encoding a VHH antibody according to claim 40 or 57 or a fusion molecule according to claim 68.

75. An expression vector comprising a nucleic acid molecule according to claim 74 operably linked to a promoter.

76. A host cell comprising a nucleic acid molecule according to claim 74 or an expression vector according to claim 75.

77. A method of treating a central nervous system disease or disorder in a patient, the method comprises administering an effective amount of a fusion molecule according to claim 71 or a pharmaceutical composition according to claim 73 to the patient, wherein the therapeutic agent is a therapeutic agent capable of treating the central nervous system disease or disorder.

78. A method of treating cancer in a patient, the method comprises administering an effective amount of a fusion molecule according to claim 71 or a pharmaceutical composition according to claim 73 to the patient, wherein the therapeutic agent is a therapeutic agent capable of treating cancer.

79. A method of treating muscular dystrophy in a patient, the method comprises administering an effective amount of a fusion molecule according to claim 71 or a pharmaceutical composition according to claim 73 to the patient, wherein the therapeutic agent is a therapeutic agent capable of treating muscular dystrophy.


SEQUENCE LISTING (87 sequences)

SEQ ID NO: 1 - protein, 7 aa, synthetic construct
  VARIANT 3: note = S, T, A
  VARIANT 4: note = G, A, H, S, T
  VARIANT 6: note = S, N, D
FTXXGXT

SEQ ID NO: 2 - protein, 8 aa, synthetic construct
  VARIANT 3: note = P, D
  VARIANT 5: note = S, T
  VARIANT 6: note = N, L, M
GTXFXXNY

SEQ ID NO: 3 - protein, 8 aa, synthetic construct
  VARIANT 6: note = L, M
GTDFSXNY

SEQ ID NO: 4 - protein, 7 aa, synthetic construct
  VARIANT 3: note = S, A
  VARIANT 4: note = S, T
FTXXGST

SEQ ID NO: 5 - protein, 5 aa, synthetic construct
HFLGD

SEQ ID NO: 6 - protein, 8 aa, synthetic construct
GTDFSLNY

SEQ ID NO: 7 - protein, 7 aa, synthetic construct
  VARIANT 4: note = S, T
FTAXGST

SEQ ID NO: 8 - protein, 7 aa, synthetic construct
FTATGST

SEQ ID NO: 9 - protein, 7 aa, synthetic construct
FTASGST

SEQ ID NO: 10 - protein, 8 aa, synthetic construct
GTDFSMNY

SEQ ID NO: 11 - protein, 7 aa, synthetic construct
FTSTGST

SEQ ID NO: 12 - protein, 5 aa, synthetic construct
  VARIANT 5: note = V, N
YFLDX

SEQ ID NO: 13 - protein, 8 aa, synthetic construct
  VARIANT 5: note = S, T
GTPFXNNY

SEQ ID NO: 14 - protein, 7 aa, synthetic construct
  VARIANT 4: note = G, A
  VARIANT 6: note = S, N
FTSXGXT

SEQ ID NO: 15 - protein, 5 aa, synthetic construct
YFLDV

SEQ ID NO: 16 - protein, 8 aa, synthetic construct
GTPFSNNY

SEQ ID NO: 17 - protein, 7 aa, synthetic construct
FTSGGST

SEQ ID NO: 18 - protein, 8 aa, synthetic construct
GTPFTNNY

SEQ ID NO: 19 - protein, 7 aa, synthetic construct
FTSAGNT

SEQ ID NO: 20 - protein, 5 aa, synthetic construct
YFLDN

SEQ ID NO: 21 - protein, 7 aa, synthetic construct
FTTHGDT

SEQ ID NO: 22 - protein, 7 aa, synthetic construct
FTSSGNT

SEQ ID NO: 23 - protein, 8 aa, synthetic construct
GNRFGIEY

SEQ ID NO: 24 - protein, 7 aa, synthetic construct
FTSAGST

SEQ ID NO: 25 - protein, 5 aa, synthetic construct
HYLGD

SEQ ID NO: 26 - protein, 25 aa, synthetic construct
  VARIANT 9: note = R, G
  VARIANT 11: note = L, V
  VARIANT 14: note = A, T
  VARIANT 23: note = A, T, V
  VARIANT 24: note = A, L, V
QVQLQESGXGXVQXGGSLRLSCXXS

SEQ ID NO: 27 - protein, 17 aa, synthetic construct
  VARIANT 1: note = M, L
  VARIANT 10: note = K, N
  VARIANT 14: note = W, F
XRWYRQAPGXQREXVAG

SEQ ID NO: 28 - protein, 38 aa, synthetic construct
  VARIANT 3: note = A, P, G
  VARIANT 6: note = V, M
  VARIANT 13: note = S, G
  VARIANT 18: note = E, K
  VARIANT 19: note = G, N
  VARIANT 27: note = N, S
  VARIANT 29: note = K, I, T
  VARIANT 37: note = Y, F
NYXDSXKGRFTIXRDNAXXTVYLQMDXLXPEDTAVYXC

SEQ ID NO: 29 - protein, 11 aa, synthetic construct
WGQGTQVTVSS

SEQ ID NO: 30 - protein, 111 aa, synthetic construct
QVQLQESGGGLVQAGGSLRLSCAASGTPFSNNYMRWYRQAPGKQREWVAGFTSGGSTNYADSVKGRFTISRDNAEGTVYLQMDNLKPEDTAVYYCYFLDVWGQGTQVTVSS

SEQ ID NO: 31 - protein, 111 aa, synthetic construct
QVQLQESGGGLVQAGGSLRLSCTASGTPFTNNYMRWYRQAPGKQREWVAGFTSAGNTNYADSVKGRFTISRDNAEGTVYLQMDNLKPEDTAVYYCYFLDVWGQGTQVTVSS

SEQ ID NO: 32 - protein, 111 aa, synthetic construct
QVQLQESGGGLVQAGGSLRLSCAASGTDFSLNYMRWYRQAPGKQREWVAGFTTHGDTNYADSMKGRFTISRDNAKNTVYLQMDSLKPEDTAVYFCYFLDNWGQGTQVTVSS

SEQ ID NO: 33 - protein, 111 aa, synthetic construct
QVQLQESGGGLVQAGGSLRLSCALSGTDFSMNYMRWYRQAPGKQREWVAGFTSSGNTNYPDSVKGRFTISRDNAKNTVYLQMDSLKPEDTAVYYCYFLDNWGQGTQVTVSS

SEQ ID NO: 34 - protein, 111 aa, synthetic construct
QVQLQESGGGLVQAGGSLRLSCVASGTDFSMNYMRWYRQAPGKQREWVAGFTSTGSTNYPDSMKGRFTISRDNAKNTVYLQMDSLKPEDTAVYYCHFLGDWGQGTQVTVSS

SEQ ID NO: 35 - protein, 111 aa, synthetic construct
QVQLQESGGGLVQTGGSLRLSCVVSGTDFSLNYLRWYRQAPGNQREWVAGFTATGSTNYPDSMKGRFTIGRDNAKNTVYLQMDSLKPEDTAVYYCHFLGDWGQGTQVTVSS

SEQ ID NO: 36 - protein, 111 aa, synthetic construct
QVQLQESGGGLVQTGGSLRLSCVVSGTDFSLNYLRWYRQAPGKQREWVAGFTASGSTNYPDSMKGRFTIGRDNAKNTVYLQMDSLTPEDTAVYYCHFLGDWGQGTQVTVSS

SEQ ID NO: 37 - protein, 111 aa, synthetic construct
QVQLQESGRGVVQAGGSLRLSCAASGNRFGIEYMRWYRQAPGKQREFVAGFTSAGSTNYGDSVKGRFTISRDNAKNTVYLQMDSLIPEDTAVYYCHYLGDWGQGTQVTVSS

SEQ ID NO: 38 - protein, 8 aa, synthetic construct
  VARIANT 5: note = S, N
DSAFXMNT

SEQ ID NO: 39 - protein, 7 aa, synthetic construct
IVSDDNT

SEQ ID NO: 40 - protein, 5 aa, synthetic construct
KGDVV

SEQ ID NO: 41 - protein, 8 aa, synthetic construct
DSAFSMNT

SEQ ID NO: 42 - protein, 8 aa, synthetic construct
DSAFNMNT

SEQ ID NO: 43 - protein, 25 aa, synthetic construct
QVQLQESGGGLVQVGGSLRLSCAAS

SEQ ID NO: 44 - protein, 17 aa, synthetic construct
  VARIANT 11: note = Q, S
  VARIANT 17: note = Y, W
MYWYRQAPGKXREFVAX

SEQ ID NO: 45 - protein, 38 aa, synthetic construct
  VARIANT 1: note = R, Q
  VARIANT 27: note = S, N
  VARIANT 35: note = V, G
XYADSVKGRFTISRDNAKNTVYLQMNXLKPEDTAXYYC

SEQ ID NO: 46 - protein, 110 aa, synthetic construct
QVQLQESGGGLVQVGGSLRLSCAASDSAFSMNTMYWYRQAPGKQREFVAYIVSDDNTRYADSVKGRFTISRDNAKNTVYLQMNSLKPEDTAVYYCKGDVVWGQGTQVTVS

SEQ ID NO: 47 - protein, 111 aa, synthetic construct
QVQLQESGGGLVQVGGSLRLSCAASDSAFNMNTMYWYRQAPGKSREFVAWIVSDDNTQYADSVKGRFTISRDNAKNTVYLQMNNLKPEDTGVYYCKGDVVWGQGTQVTVSS

SEQ ID NO: 48 - protein, 649 aa, Homo sapiens
AHHHHHHSGRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 49 - protein, 650 aa, Mus musculus
AHHHHHHGSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 50 - protein, 649 aa, Macaca fascicularis
AHHHHHHSGRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 51 - protein, 19 aa, synthetic construct
MDWLRNLLFLMAAAQSINA

SEQ ID NO: 52 - DNA, 23 bp, synthetic construct
  source 1..23: mol_type = other DNA
gtcctggctgctcttctacaagg

SEQ ID NO: 53 - DNA, 23 bp, synthetic construct
  source 1..23: mol_type = other DNA
ggtacgtgctgttgaactgttcc

SEQ ID NO: 54 - DNA, 51 bp, synthetic construct
  source 1..51: mol_type = other DNA
aaaggcccagccggccatggcgcaggtgcagctgcaggagtctggrggagg

SEQ ID NO: 55 - DNA, 43 bp, synthetic construct
  source 1..43: mol_type = other DNA
aaaggcctcccgggccacgttttgaggagacggtgacctgggt

SEQ ID NO: 56 - protein, 6 aa, synthetic construct
HHHHHH

SEQ ID NO: 57 - protein, 4 aa, synthetic construct
EPEA

SEQ ID NO: 58 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 340 aa, synthetic construct
QVQLQESGGGLVQAGGSLRLSCAASGTPFSNNYMRWYRQAPGKQREWVAGFTSGGSTNYADSVKGRFTISRDNAEGTVYLQMDNLKPEDTAVYYCYFLDVWGQGTQVTVSSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 340 aa, synthetic construct
QVQLQESGGGLVQAGGSLRLSCTASGTPFTNNYMRWYRQAPGKQREWVAGFTSAGNTNYADSVKGRFTISRDNAEGTVYLQMDNLKPEDTAVYYCYFLDVWGQGTQVTVSSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 340 aa, synthetic construct
QVQLQESGGGLVQAGGSLRLSCAASGTDFSLNYMRWYRQAPGKQREWVAGFTTHGDTNYADSMKGRFTISRDNAKNTVYLQMDSLKPEDTAVYFCYFLDNWGQGTQVTVSSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 340 aa, synthetic construct
QVQLQESGGGLVQAGGSLRLSCALSGTDFSMNYMRWYRQAPGKQREWVAGFTSSGNTNYPDSVKGRFTISRDNAKNTVYLQMDSLKPEDTAVYYCYFLDNWGQGTQVTVSSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 340 aa, synthetic construct
QVQLQESGGGLVQAGGSLRLSCVASGTDFSMNYMRWYRQAPGKQREWVAGFTSTGSTNYPDSMKGRFTISRDNAKNTVYLQMDSLKPEDTAVYYCHFLGDWGQGTQVTVSSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 340 aa, synthetic construct
QVQLQESGGGLVQTGGSLRLSCVVSGTDFSLNYLRWYRQAPGNQREWVAGFTATGSTNYPDSMKGRFTIGRDNAKNTVYLQMDSLKPEDTAVYYCHFLGDWGQGTQVTVSSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 340 aa, synthetic construct
QVQLQESGGGLVQTGGSLRLSCVVSGTDFSLNYLRWYRQAPGKQREWVAGFTASGSTNYPDSMKGRFTIGRDNAKNTVYLQMDSLTPEDTAVYYCHFLGDWGQGTQVTVSSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 340 aa, synthetic construct
QVQLQESGRGVVQAGGSLRLSCAASGNRFGIEYMRWYRQAPGKQREFVAGFTSAGSTNYGDSVKGRFTISRDNAKNTVYLQMDSLIPEDTAVYYCHYLGDWGQGTQVTVSSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 340 aa, synthetic construct
QVQLQESGGGLVQVGGSLRLSCAASDSAFSMNTMYWYRQAPGKQREFVAYIVSDDNTRYADSVKGRFTISRDNAKNTVYLQMNSLKPEDTAVYYCKGDVVWGQGTQVTVSSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 118 aa, synthetic construct
EVKLVESGGGLVKPGASLKLSCAASGFTFSNYGMSWVRQNSDKRLEWVASIRSGGGRTYYSDNVKGRFTISRENAKNTLYLQMSSLKSEDTALYYCVRYDHYSGSSDYWGQGTTVTVS

SEQ ID NO: 69 - protein, 112 aa, synthetic construct
YVVMTQTPLTLSVTIGQPASISCKSSQSLLDSDGKTYLNWLLQRPGQSPKRLIYLVSKLDSGVPDRFTGSGSGTDFTLKISRIEAEDLGLYYCWQGTHFPRTFGGGTKLEIK

SEQ ID NO: 70 - protein, 15 aa, synthetic construct
GGGGSGGGGSGGGGS

SEQ ID NO: 71 - protein, 386 aa, synthetic construct
QVQLQESGGGLVQAGGSLRLSCAASGTPFSNNYMRWYRQAPGKQREWVAGFTSGGSTNYADSVKGRFTISRDNAEGTVYLQMDNLKPEDTAVYYCYFLDVWGQGTQVTVSSGGGGSGGGGSGGGGSEVKLVESGGGLVKPGASLKLSCAASGFTFSNYGMSWVRQNSDKRLEWVASIRSGGGRTYYSDNVKGRFTISRENAKNTLYLQMSSLKSEDTALYYCVRYDHYSGSSDYWGQGTTVTVSGGGGSGGGGSGGGGSYVVMTQTPLTLSVTIGQPASISCKSSQSLLDSDGKTYLNWLLQRPGQSPKRLIYLVSKLDSGVPDRFTGSGSGTDFTLKISRIEAEDLGLYYCWQGTHFPRTFGGGTKLEIKGSENLYFQSHHHHHH

SEQ ID NO: 72 - protein, 386 aa, synthetic construct
EVKLVESGGGLVKPGASLKLSCAASGFTFSNYGMSWVRQNSDKRLEWVASIRSGGGRTYYSDNVKGRFTISRENAKNTLYLQMSSLKSEDTALYYCVRYDHYSGSSDYWGQGTTVTVSGGGGSGGGGSGGGGSYVVMTQTPLTLSVTIGQPASISCKSSQSLLDSDGKTYLNWLLQRPGQSPKRLIYLVSKLDSGVPDRFTGSGSGTDFTLKISRIEAEDLGLYYCWQGTHFPRTFGGGTKLEIKGGGGSGGGGSGGGGSQVQLQESGGGLVQAGGSLRLSCAASGTPFSNNYMRWYRQAPGKQREWVAGFTSGGSTNYADSVKGRFTISRDNAEGTVYLQMDNLKPEDTAVYYCYFLDVWGQGTQVTVSSGSENLYFQSHHHHHH

SEQ ID NO: 73 - protein, 386 aa, synthetic construct
EVQLVESGGGVVQPGGSLKLSCVASGTDFSINFIRWYRQAPGKQREFVAGFTATGNTNYADSMKGRFTISRDNTKNAVYLQIDSLKPEDTAVYYCYMLDKWGQGTQVTVSSGGGGSGGGGSGGGGSEVKLVESGGGLVKPGASLKLSCAASGFTFSNYGMSWVRQNSDKRLEWVASIRSGGGRTYYSDNVKGRFTISRENAKNTLYLQMSSLKSEDTALYYCVRYDHYSGSSDYWGQGTTVTVSGGGGSGGGGSGGGGSYVVMTQTPLTLSVTIGQPASISCKSSQSLLDSDGKTYLNWLLQRPGQSPKRLIYLVSKLDSGVPDRFTGSGSGTDFTLKISRIEAEDLGLYYCWQGTHFPRTFGGGTKLEIKGSENLYFQSHHHHHH

SEQ ID NO: 74 - protein, 386 aa, synthetic construct
EVKLVESGGGLVKPGASLKLSCAASGFTFSNYGMSWVRQNSDKRLEWVASIRSGGGRTYYSDNVKGRFTISRENAKNTLYLQMSSLKSEDTALYYCVRYDHYSGSSDYWGQGTTVTVSGGGGSGGGGSGGGGSYVVMTQTPLTLSVTIGQPASISCKSSQSLLDSDGKTYLNWLLQRPGQSPKRLIYLVSKLDSGVPDRFTGSGSGTDFTLKISRIEAEDLGLYYCWQGTHFPRTFGGGTKLEIKGGGGSGGGGSGGGGSEVQLVESGGGVVQPGGSLKLSCVASGTDFSINFIRWYRQAPGKQREFVAGFTATGNTNYADSMKGRFTISRDNTKNAVYLQIDSLKPEDTAVYYCYMLDKWGQGTQVTVSSGSENLYFQSHHHHHH

SEQ ID NO: 75 - protein, 667 aa, synthetic construct
EVQLVQSGAEVKKPGESLKISCKGSGYSFTNYWLGWVRQMPGKGLEWMGDIYPGGDYPTYSEKFKVQVTISADKSISTAYLQWSSLKASDTAMYYCARSGNYDEVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQSTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 661 aa, synthetic construct
EVQLQQSGPELVKPGASMKISCKASGYSFTGYTMNWVKQSHGENLEWIGRINPHNGGTDYNQKFKDKAPLTVDKSSNTAYMELLSLTSEDSAVYYCARGYYYYSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKQIVLTQSPAIMSASPGEKVTMTCSASSSIDYIHWYQQKSGTSPKRWIYDTSKLASGVPARFSGSGSGTSYSLTISSMEPEDAATYYCHQRNSYPWTFGGGTRLEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 25 aa, synthetic construct
  VARIANT 14: note = A, T
  VARIANT 23: note = A, T, V
  VARIANT 24: note = A, L, V
QVQLQESGGGLVQXGGSLRLSCXXS

SEQ ID NO: 78 - protein, 17 aa, synthetic construct
  VARIANT 1: note = M, L
  VARIANT 10: note = K, N
XRWYRQAPGXQREWVAG

SEQ ID NO: 79 - protein, 19 aa, Mus musculus
MGWSCIILFLVATATGVHS

SEQ ID NO: 80 - protein, 111 aa, synthetic construct
EVQLVESGGGVVQPGGSLKLSCVASGTDFSINFIRWYRQAPGKQREFVAGFTATGNTNYADSMKGRFTISRDNTKNAVYLQIDSLKPEDTAVYYCYMLDKWGQGTQVTVSS

SEQ ID NO: 81 - protein, 19 aa, Mus musculus
METDTLLLWVLLLWVPGST

SEQ ID NO: 82 - protein, 340 aa, synthetic construct
QVQLQESGGGLVQVGGSLRLSCAASDSAFNMNTMYWYRQAPGKSREFVAWIVSDDNTQYADSVKGRFTISRDNAKNTVYLQMNNLKPEDTGVYYCKGDVVWGQGTQVTVSSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 111 aa, synthetic construct
QVQLQESGGGLVQAGGSLRLSCAASGTPFSNNYMRWYRQAPGKQREWVAGFTSGGSTNYADSVKGRFTISRDNAKGTVYLQMNSLKPEDTAVYYCYFLDVWGQGTQVTVSS

SEQ ID NO: 84 - protein, 111 aa, synthetic construct
QVQLQESGGGLVQAGGSLRLSCAASGTDFSLNYMRWYRQAPGKQREWVAGFTTHGDTNYADSMKGRFTISRDNAKNTVYLQMNSLKPEDTAVYFCYFLDNWGQGTQVTVSS

SEQ ID NO: 85 - protein, 371 aa, synthetic construct
QVQLQESGGGLVQAGGSLRLSCAASGTDFSLNYMRWYRQAPGKQREWVAGFTTHGDTNYADSMKGRFTISRDNAKNTVYLQMNSLKPEDTAVYFCYFLDNWGQGTQVTVSSGGGGSGGGGSGGGGSEVKLVESGGGLVKPGASLKLSCAASGFTFSNYGMSWVRQNSDKRLEWVASIRSGGGRTYYSDNVKGRFTISRENAKNTLYLQMSSLKSEDTALYYCVRYDHYSGSSDYWGQGTTVTVSGGGGSGGGGSGGGGSYVVMTQTPLTLSVTIGQPASISCKSSQSLLDSDGKTYLNWLLQRPGQSPKRLIYLVSKLDSGVPDRFTGSGSGTDFTLKISRIEAEDLGLYYCWQGTHFPRTFGGGTKLEIK

SEQ ID NO: 86 - protein, 8 aa, synthetic construct
GGHHHHHH

SEQ ID NO: 87 - protein, 132 aa, synthetic construct
QVQLQESGGGLVQPGGSLRLSCAASGRTFSSYAMGWFRQAPGKQREFVAAIRWSGGYTYYTDSVKGRFTISRDNAKTTVYLQMNSLKPEDTAVYYCAATYLSSDYSRYALPQRPLDYDYWGQGTQVTVSSLE